SEQUENCE LISTING (27 sequences)

SEQ ID NO: 1 - DNA, 3633 bp, Homo sapiens
  CDS (1)..(3630)
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 2 - protein, 1210 aa, Homo sapiens [sequence omitted]

SEQ ID NO: 3 - DNA, 1974 bp, Homo sapiens
  CDS (1)..(1971)
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatggaagctacatagtgtctcactttccaagatcattctacaagatgtcagtgcactga

SEQ ID NO: 4 - protein, 657 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGSYIVSHFPRSFYKMSVH

SEQ ID NO: 5 - DNA, 2118 bp, Homo sapiens
  CDS (1)..(2115)
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacgggccaggaaatgagagtctcaaagccatgttattctgcctttttaaactatcatcctgtaatcaaagtaatgatggcagcgtgtcccaccagagcgggagcccagctgctcaggagtcatgcttaggatggatcccttctcttctgccgtcagagtttcagctgggttggggtggatgcagccacctccatgcctggccttctgcatctgtgatcatcacggcctcctcctgccactga

SEQ ID NO: 6 - protein, 705 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGPGNESLKAMLFCLFKLSSCNQSNDGSVSHQSGSPAAQESCLGWIPSLLPSEFQLGWGGCSHLHAWPSASVIITASSCH

SEQ ID NO: 7 - DNA, 1887 bp, Homo sapiens
  CDS (1)..(1884)
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacgggtcctaa

SEQ ID NO: 8 - protein, 628 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGS

SEQ ID NO: 9 - DNA, 1218 bp, Homo sapiens
  CDS (1)..(1215)
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacaggtttgagctga

SEQ ID NO: 10 - protein, 405 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGLS

SEQ ID NO: 11 - DNA, 2829 bp, Homo sapiens
  CDS (1)..(2826)
atgcgaccctccgggacggccggggcagcgctcctggcgctgctggctgcgctctgcccggcgagtcgggctctggaggaaaagcgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 12 - protein, 942 aa, Homo sapiens
MRPSGTAGAALLALLAALCPASRALEEKRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 13 - DNA, 3633 bp, Mus musculus
  CDS (1)..(3630)
atgcgaccctcagggaccgcgagaaccacactgctggtgctgctgaccgcgctctgcgccgcaggtggggcgttggaggaaaagaaagtctgccaaggcacaagtaacaggctcacccaactgggcacttttgaagaccactttctgagcctgcagaggatgtacaacaactgtgaagtggtccttgggaacttggaaattacctatgtgcaaaggaattacgacctttccttcttaaagaccatccaggaggtggccggctatgtcctcattgccctcaacaccgtggagagaatccctttggagaacctgcagatcatcaggggaaatgctctttatgaaaacacctatgccttagccatcctgtccaactatgggacaaacagaactgggcttagggaactgcccatgcggaacttacaggaaatcctgattggtgctgtgcgattcagcaacaaccccatcctctgcaatatggatactatccagtggagggacatcgtccaaaacgtctttatgagcaacatgtcaatggacttacagagccatccgagcagttgccccaaatgtgatccaagctgtcccaatggaagctgctggggaggaggagaggagaactgccagaaattgaccaaaatcatctgtgcccagcaatgttcccatcgctgtcgtggcaggtcccccagtgactgctgccacaaccaatgtgctgcggggtgtacagggccccgagagagtgactgtctggtctgccaaaagttccaagatgaggccacatgcaaagacacctgcccaccactcatgctgtacaaccccaccacctatcagatggatgtcaaccctgaagggaagtacagctttggtgccacctgtgtgaagaagtgcccccgaaactacgtggtgacagatcatggctcatgtgtccgagcctgtgggcctgactactacgaagtggaagaagatggcatccgcaagtgtaaaaaatgtgatgggccctgtcgcaaagtttgtaatggcataggcattggtgaatttaaagacacactctccataaatgctacaaacatcaaacacttcaaatactgcactgccatcagcggggaccttcacatcctgccagtggcctttaagggggattctttcacgcgcactcctcctctagacccacgagaactagaaattctaaaaaccgtaaaggaaataacaggctttttgctgattcaggcttggcctgataactggactgacctccatgctttcgagaacctagaaataatacgtggcagaacaaagcaacatggtcagttttctttggcggtcgttggcctgaacatcacatcactggggctgcgttccctcaaggagatcagtgatggggatgtgatcatttctggaaaccgaaatttgtgctacgcaaacacaataaactggaaaaaactcttcgggacacccaatcagaaaaccaaaatcatgaacaacagagctgagaaagactgcaaggccgtgaaccacgtctgcaatcctttatgctcctcggaaggctgctggggccctgagcccagggactgtgtctcctgccagaatgtgagcagaggcagggagtgcgtggagaaatgcaacatcctggagggggaaccaagggagtttgtggaaaattctgaatgcatccagtgccatccagaatgtctgccccaggccatgaacatcacctgtacaggcagggggccagacaactgcatccagtgtgcccactacattgatggcccacactgtgtcaagacctgcccagctggcatcatgggagagaacaacactctggtctggaagtatgcagatgccaataatgtctgccacctatgccacgccaactgtacctatggatgtgctgggccaggtcttcaaggatgtgaagtgtggccatctgggccaaagataccatctattgccactgggattgtgggtggcctcctcttcatagtggtggtggcccttgggattggcctattcatgcgaagacgtcacattgttcgaaagcgtacactacgccgcctgcttcaagagagagagctcgtggaacctctcacacccagcggagaagctccaaaccaagcccacttgaggatattaaaggaaacagaattcaaaaagatcaaagttctgggttcgggagcatttggcacagtgtataagggtctctggatcccagaaggtgagaaagtaaaaatcccggtggccatcaaggagttaagagaagccacatctccaaaagccaacaaagaaatccttgacgaagcctatgtgatggctagtgtggacaaccctcatgtatgccgcctcctgggcatctgtctgacctccactgtccagctcattacacagctcatgccctacggttgcctcctggactacgtccgagaacacaaggacaacattggctcccagtacctcctcaactggtgtgtgcagattgcaaagggcatgaactacctggaagatcggcgtttggtgcaccgtgacttggcagccaggaatgtactggtgaagacaccacagcatgtcaagatcacagattttgggctggccaaactgcttggtgctgaagagaaagaatatcatgccgaggggggcaaagtgcctatcaagtggatggctttggaatcaattttacaccgaatttatacacaccaaagtgatgtctggagctatggtgtcactgtgtgggaactgatgacctttgggtccaagccttatgatggaatcccagcaagtgacatctcatccatcctagagaaaggagaacgccttccacagccacctatctgcaccatcgatgtctacatgatcatggtcaagtgctggatgatagatgctgatagccgcccaaagttccgagagttgattcttgaattctccaaaatggcccgagacccacagcgctaccttgttatccagggggatgaaagaatgcatttgccaagccctacagactccaacttttaccgagccctgatggatgaagaggacatggaggatgtagttgatgctgatgagtatcttaccccacagcaaggcttcttcaacagcccgtccacgtcgaggactcccctcttgagttctctgagtgcaactagcaacaattccactgtggcttgcattaatagaaatgggagctgccgtgtcaaagaagacgccttcttgcagcggtacagctccgaccccacaggtgctgtaacagaggacaacatagatgacgcattcctccctgtacctgaatatgtaaaccaatctgttcccaagaggccagcaggctctgtgcagaaccctgtctatcacaatcagcccctgcatccagctcctggaagagacctgcattatcaaaatccccacagcaatgcagtgggcaaccctgagtatctcaacactgcccagcctacctgtctcagtagtgggtttaacagccctgcactctggatccagaaaggcagtcaccaaatgagcctagacaaccctgactaccagcaggacttcttccccaaggaaaccaagccaaatggcatatttaagggccccacagctgaaaatgcagagtacctacgggtggcacctccaagcagcgagtttattggagcatga

SEQ ID NO: 14 - protein, 1210 aa, Mus musculus
MRPSGTARTTLLVLLTALCAAGGALEEKKVCQGTSNRLTQLGTFEDHFLSLQRMYNNCEVVLGNLEITYVQRNYDLSFLKTIQEVAGYVLIALNTVERIPLENLQIIRGNALYENTYALAILSNYGTNRTGLRELPMRNLQEILIGAVRFSNNPILCNMDTIQWRDIVQNVFMSNMSMDLQSHPSSCPKCDPSCPNGSCWGGGEENCQKLTKIICAQQCSHRCRGRSPSDCCHNQCAAGCTGPRESDCLVCQKFQDEATCKDTCPPLMLYNPTTYQMDVNPEGKYSFGATCVKKCPRNYVVTDHGSCVRACGPDYYEVEEDGIRKCKKCDGPCRKVCNGIGIGEFKDTLSINATNIKHFKYCTAISGDLHILPVAFKGDSFTRTPPLDPRELEILKTVKEITGFLLIQAWPDNWTDLHAFENLEIIRGRTKQHGQFSLAVVGLNITSLGLRSLKEISDGDVIISGNRNLCYANTINWKKLFGTPNQKTKIMNNRAEKDCKAVNHVCNPLCSSEGCWGPEPRDCVSCQNVSRGRECVEKCNILEGEPREFVENSECIQCHPECLPQAMNITCTGRGPDNCIQCAHYIDGPHCVKTCPAGIMGENNTLVWKYADANNVCHLCHANCTYGCAGPGLQGCEVWPSGPKIPSIATGIVGGLLFIVVVALGIGLFMRRRHIVRKRTLRRLLQERELVEPLTPSGEAPNQAHLRILKETEFKKIKVLGSGAFGTVYKGLWIPEGEKVKIPVAIKELREATSPKANKEILDEAYVMASVDNPHVCRLLGICLTSTVQLITQLMPYGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASDISSILEKGERLPQPPICTIDVYMIMVKCWMIDADSRPKFRELILEFSKMARDPQRYLVIQGDERMHLPSPTDSNFYRALMDEEDMEDVVDADEYLTPQQGFFNSPSTSRTPLLSSLSATSNNSTVACINRNGSCRVKEDAFLQRYSSDPTGAVTEDNIDDAFLPVPEYVNQSVPKRPAGSVQNPVYHNQPLHPAPGRDLHYQNPHSNAVGNPEYLNTAQPTCLSSGFNSPALWIQKGSHQMSLDNPDYQQDFFPKETKPNGIFKGPTAENAEYLRVAPPSSEFIGA

SEQ ID NO: 15 - DNA, 1932 bp, Mus musculus
  CDS (1)..(1929)
atgcgaccctcagggaccgcgagaaccacactgctggtgctgctgaccgcgctctgcgccgcaggtggggcgttggaggaaaagaaagtctgccaaggcacaagtaacaggctcacccaactgggcacttttgaagaccactttctgagcctgcagaggatgtacaacaactgtgaagtggtccttgggaacttggaaattacctatgtgcaaaggaattacgacctttccttcttaaagaccatccaggaggtggccggctatgtcctcattgccctcaacaccgtggagagaatccctttggagaacctgcagatcatcaggggaaatgctctttatgaaaacacctatgccttagccatcctgtccaactatgggacaaacagaactgggcttagggaactgcccatgcggaacttacaggaaatcctgattggtgctgtgcgattcagcaacaaccccatcctctgcaatatggatactatccagtggagggacatcgtccaaaacgtctttatgagcaacatgtcaatggacttacagagccatccgagcagttgccccaaatgtgatccaagctgtcccaatggaagctgctggggaggaggagaggagaactgccagaaattgaccaaaatcatctgtgcccagcaatgttcccatcgctgtcgtggcaggtcccccagtgactgctgccacaaccaatgtgctgcggggtgtacagggccccgagagagtgactgtctggtctgccaaaagttccaagatgaggccacatgcaaagacacctgcccaccactcatgctgtacaaccccaccacctatcagatggatgtcaaccctgaagggaagtacagctttggtgccacctgtgtgaagaagtgcccccgaaactacgtggtgacagatcatggctcatgtgtccgagcctgtgggcctgactactacgaagtggaagaagatggcatccgcaagtgtaaaaaatgtgatgggccctgtcgcaaagtttgtaatggcataggcattggtgaatttaaagacacactctccataaatgctacaaacatcaaacacttcaaatactgcactgccatcagcggggaccttcacatcctgccagtggcctttaagggggattctttcacgcgcactcctcctctagacccacgagaactagaaattctaaaaaccgtaaaggaaataacaggctttttgctgattcaggcttggcctgataactggactgacctccatgctttcgagaacctagaaataatacgtggcagaacaaagcaacatggtcagttttctttggcggtcgttggcctgaacatcacatcactggggctgcgttccctcaaggagatcagtgatggggatgtgatcatttctggaaaccgaaatttgtgctacgcaaacacaataaactggaaaaaactcttcgggacacccaatcagaaaaccaaaatcatgaacaacagagctgagaaagactgcaaggccgtgaaccacgtctgcaatcctttatgctcctcggaaggctgctggggccctgagcccagggactgtgtctcctgccagaatgtgagcagaggcagggagtgcgtggagaaatgcaacatcctggagggggaaccaagggagtttgtggaaaattctgaatgcatccagtgccatccagaatgtctgccccaggccatgaacatcacctgtacaggcagggggccagacaactgcatccagtgtgcccactacattgatggcccacactgtgtcaagacctgcccagctggcatcatgggagagaacaacactctggtctggaagtatgcagatgccaataatgtctgccacctatgccacgccaactgtacctatggagtaatggttcctgaaatgttgctccaaagtatcattttaaaacctatttga

SEQ ID NO: 16 - protein, 643 aa, Mus musculus
MRPSGTARTTLLVLLTALCAAGGALEEKKVCQGTSNRLTQLGTFEDHFLSLQRMYNNCEVVLGNLEITYVQRNYDLSFLKTIQEVAGYVLIALNTVERIPLENLQIIRGNALYENTYALAILSNYGTNRTGLRELPMRNLQEILIGAVRFSNNPILCNMDTIQWRDIVQNVFMSNMSMDLQSHPSSCPKCDPSCPNGSCWGGGEENCQKLTKIICAQQCSHRCRGRSPSDCCHNQCAAGCTGPRESDCLVCQKFQDEATCKDTCPPLMLYNPTTYQMDVNPEGKYSFGATCVKKCPRNYVVTDHGSCVRACGPDYYEVEEDGIRKCKKCDGPCRKVCNGIGIGEFKDTLSINATNIKHFKYCTAISGDLHILPVAFKGDSFTRTPPLDPRELEILKTVKEITGFLLIQAWPDNWTDLHAFENLEIIRGRTKQHGQFSLAVVGLNITSLGLRSLKEISDGDVIISGNRNLCYANTINWKKLFGTPNQKTKIMNNRAEKDCKAVNHVCNPLCSSEGCWGPEPRDCVSCQNVSRGRECVEKCNILEGEPREFVENSECIQCHPECLPQAMNITCTGRGPDNCIQCAHYIDGPHCVKTCPAGIMGENNTLVWKYADANNVCHLCHANCTYGVMVPEMLLQSIILKPI

SEQ ID NO: 17 - DNA, 1968 bp, Mus musculus
  CDS (1)..(1965)
atgcgaccctcagggaccgcgagaaccacactgctggtgctgctgaccgcgctctgcgccgcaggtggggcgttggaggaaaagaaagtctgccaaggcacaagtaacaggctcacccaactgggcacttttgaagaccactttctgagcctgcagaggatgtacaacaactgtgaagtggtccttgggaacttggaaattacctatgtgcaaaggaattacgacctttccttcttaaagaccatccaggaggtggccggctatgtcctcattgccctcaacaccgtggagagaatccctttggagaacctgcagatcatcaggggaaatgctctttatgaaaacacctatgccttagccatcctgtccaactatgggacaaacagaactgggcttagggaactgcccatgcggaacttacaggaaatcctgattggtgctgtgcgattcagcaacaaccccatcctctgcaatatggatactatccagtggagggacatcgtccaaaacgtctttatgagcaacatgtcaatggacttacagagccatccgagcagttgccccaaatgtgatccaagctgtcccaatggaagctgctggggaggaggagaggagaactgccagaaattgaccaaaatcatctgtgcccagcaatgttcccatcgctgtcgtggcaggtcccccagtgactgctgccacaaccaatgtgctgcggggtgtacagggccccgagagagtgactgtctggtctgccaaaagttccaagatgaggccacatgcaaagacacctgcccaccactcatgctgtacaaccccaccacctatcagatggatgtcaaccctgaagggaagtacagctttggtgccacctgtgtgaagaagtgcccccgaaactacgtggtgacagatcatggctcatgtgtccgagcctgtgggcctgactactacgaagtggaagaagatggcatccgcaagtgtaaaaaatgtgatgggccctgtcgcaaagtttgtaatggcataggcattggtgaatttaaagacacactctccataaatgctacaaacatcaaacacttcaaatactgcactgccatcagcggggaccttcacatcctgccagtggcctttaagggggattctttcacgcgcactcctcctctagacccacgagaactagaaattctaaaaaccgtaaaggaaataacaggctttttgctgattcaggcttggcctgataactggactgacctccatgctttcgagaacctagaaataatacgtggcagaacaaagcaacatggtcagttttctttggcggtcgttggcctgaacatcacatcactggggctgcgttccctcaaggagatcagtgatggggatgtgatcatttctggaaaccgaaatttgtgctacgcaaacacaataaactggaaaaaactcttcgggacacccaatcagaaaaccaaaatcatgaacaacagagctgagaaagactgcaaggccgtgaaccacgtctgcaatcctttatgctcctcggaaggctgctggggccctgagcccagggactgtgtctcctgccagaatgtgagcagaggcagggagtgcgtggagaaatgcaacatcctggagggggaaccaagggagtttgtggaaaattctgaatgcatccagtgccatccagaatgtctgccccaggccatgaacatcacctgtacaggcagggggccagacaactgcatccagtgtgcccactacattgatggcccacactgtgtcaagacctgcccagctggcatcatgggagagaacaacactctggtctggaagtatgcagatgccaataatgtctgccacctatgccacgccaactgtacctatggatgtgctgggccaggtcttcaaggatgtgaagtgtggccatctgggtacgttcaatggcagtggatcttaaagaccttttggatctaa

SEQ ID NO: 18 - protein, 655 aa, Mus musculus
MRPSGTARTTLLVLLTALCAAGGALEEKKVCQGTSNRLTQLGTFEDHFLSLQRMYNNCEVVLGNLEITYVQRNYDLSFLKTIQEVAGYVLIALNTVERIPLENLQIIRGNALYENTYALAILSNYGTNRTGLRELPMRNLQEILIGAVRFSNNPILCNMDTIQWRDIVQNVFMSNMSMDLQSHPSSCPKCDPSCPNGSCWGGGEENCQKLTKIICAQQCSHRCRGRSPSDCCHNQCAAGCTGPRESDCLVCQKFQDEATCKDTCPPLMLYNPTTYQMDVNPEGKYSFGATCVKKCPRNYVVTDHGSCVRACGPDYYEVEEDGIRKCKKCDGPCRKVCNGIGIGEFKDTLSINATNIKHFKYCTAISGDLHILPVAFKGDSFTRTPPLDPRELEILKTVKEITGFLLIQAWPDNWTDLHAFENLEIIRGRTKQHGQFSLAVVGLNITSLGLRSLKEISDGDVIISGNRNLCYANTINWKKLFGTPNQKTKIMNNRAEKDCKAVNHVCNPLCSSEGCWGPEPRDCVSCQNVSRGRECVEKCNILEGEPREFVENSECIQCHPECLPQAMNITCTGRGPDNCIQCAHYIDGPHCVKTCPAGIMGENNTLVWKYADANNVCHLCHANCTYGCAGPGLQGCEVWPSGYVQWQWILKTFWI

SEQ ID NO: 19 - DNA, 1962 bp, Gallus domesticus
  CDS (1)..(1962)
atgggtgtacgcagccccctgtccgcctctgggcctcgcggggccgctgtcctggtgctactgctgctgggcgtcgcgctgtgttccgccgtggaggagaagaaagtttgtcaagggacaaataacaagttgacccagctggggcacgtggaagaccatttcaccagcctgcagagaatgtacaacaactgcgaagtggtactgagtaacctggagattacctacgtggagcataatcgcgatctcaccttccttaagaccatacaggaggttgcaggctatgtgctcattgcgcttaacatggtggacgtcattcccttagaaaacctccagattatccgagggaatgtgctttatgacaactcttttgccctggcagttttatccaattaccacatgaataaaacccagggacttcgagagctgccaatgaaacggctatcagaaattctcaatggaggtgttaaaatcagcaacaaccccaaactgtgcaacatggacactgttctctggaatgacatcattgatacaagcaggaagcctctcacagtacttgactttgcaagcaatctttcttcttgtccaaaatgccatccgaactgcacagaagaccactgctggggtgctggtgaacagaactgccagactttaacaaaagtcatctgtgcccagcaatgctctggccggtgcagaggaaaggtgcccagtgactgctgccacaatcagtgtgctgcagggtgcacaggacctcgggagagtgactgcctggcatgccgcaagtttcgggatgatgctacctgcaaggacacatgtcccccactggtcctctataaccccaccacctatcaaatggatgtcaaccctgagggaaaatacagctttggagccacttgtgtgagggaatgtccacacaactatgtggtgacagatcatggctcctgcgttcgctcgtgtaatactgatacttacgaagtggaagaaaatggtgttcggaagtgtaaaaaatgtgatgggctatgtagcaaagtgtgcaatggcattggaataggtgaacttaaagggatcctatccataaatgccacaaacatcgactccttcaaaaactgtacgaagatcaatggggatgtcagcattcttcctgttgcatttctaggggatgccttcacaaagacactaccccttgaccctaagaagctggatgtctttagaacagtcaaagaaatatcaggatttttgttgattcaggcctggcctgataatgctactgatctctatgcttttgaaaatctggagattatccgaggccgaaccaagcagcacggccagtattcccttgctgttgttaacttgaaaatacagtcgttggggctgcgctccctcaaggaaataagtgatggagacattgccattatgaagaacaagaacctctgctatgctgacaccatgaactggcgcagcttgtttgctactcagagtcagaaaacaaaaattatacagaacagaaataaaaatgattgtactgctgacaggcatgtgtgtgacccgctgtgctcggacgtgggctgctggggcccagggcccttccactgcttttcctgcaggtttttcagtcgccagaaggagtgtgtaaaacagtgcaacatcctgcaaggggagccacgtgagtttgaaagagactccaaatgcctcccctgccactcagagtgtctggtacagaactccactgcatacaacacaacctgctctggaccgggcccagaccactgcatgaagtgtgcccattttatagatggtccccactgtgtgaaggcctgccccgctggggtcctgggtgagaatgataccctggtctggaagtatgcagatgccaatgctgtttgccagctctgccatccaaactgtacacgagggtgcaaagggccaggtcttgaaggatgtccaaatggctccaaaactccatct

SEQ ID NO: 20 - protein, 654 aa, Gallus domesticus
MGVRSPLSASGPRGAAVLVLLLLGVALCSAVEEKKVCQGTNNKLTQLGHVEDHFTSLQRMYNNCEVVLSNLEITYVEHNRDLTFLKTIQEVAGYVLIALNMVDVIPLENLQIIRGNVLYDNSFALAVLSNYHMNKTQGLRELPMKRLSEILNGGVKISNNPKLCNMDTVLWNDIIDTSRKPLTVLDFASNLSSCPKCHPNCTEDHCWGAGEQNCQTLTKVICAQQCSGRCRGKVPSDCCHNQCAAGCTGPRESDCLACRKFRDDATCKDTCPPLVLYNPTTYQMDVNPEGKYSFGATCVRECPHNYVVTDHGSCVRSCNTDTYEVEENGVRKCKKCDGLCSKVCNGIGIGELKGILSINATNIDSFKNCTKINGDVSILPVAFLGDAFTKTLPLDPKKLDVFRTVKEISGFLLIQAWPDNATDLYAFENLEIIRGRTKQHGQYSLAVVNLKIQSLGLRSLKEISDGDIAIMKNKNLCYADTMNWRSLFATQSQKTKIIQNRNKNDCTADRHVCDPLCSDVGCWGPGPFHCFSCRFFSRQKECVKQCNILQGEPREFERDSKCLPCHSECLVQNSTAYNTTCSGPGPDHCMKCAHFIDGPHCVKACPAGVLGENDTLVWKYADANAVCQLCHPNCTRGCKGPGLEGCPNGSKTPS

SEQ ID NO: 21 - DNA, 4134 bp, Drosophila melanogaster
  CDS (1)..(3630)
atgatgattatcagcatgtggatgagcatatcgcgaggattgtgggacagcagctccatcttgtcggtgctgctgatcctcgcctgcatggcatccatcaccacaagctcatcggtcagcaatgccggctatgtggataatggcaatatgaaagtctgcatcggcactaaatctcggctctccgtgccctccaacaaggaacatcattaccgaaacctcagagatcggtacacgaactgtacgtatgtggatggcaacttgaaactgacctggctacccaacgagaatttggacctcagcttcctagacaacatacgggaggtcaccggctatattctgatcagtcatgtggacgttaagaaagtggtgtttcccaaactacaaatcattcgcggacgcacgctgttcagcttatccgtggaggaggagaagtatgccttgttcgtcacttattccaaaatgtacacgctggagattcccgatctacgcgatgtcttaaatggccaagtgggcttccacaacaactacaatctctgccacatgcgaacgatccagtggtcggagattgtatccaacggcacggatgcatactacaactacgactttactgctccggagcgcgagtgtcccaagtgccacgagagctgcacgcacggatgttggggcgagggtcccaagaattgccagaagttcagcaagctcacctgctcgccacagtgtgccggaggtcgttgctatggaccaaagccgcgggagtgttgtcacctcttctgcgccggaggatgcactggtcccacgcaaaaggattgcatcgcctgcaagaacttcttcgacgaggcagtatcaaaggaggaatgcccgcccatgcgcaagtacaatcccaccacctatgttcttgaaacgaatcctgagggaaagtatgcctatggtgccacctgcgtcaaggagtgtcccggtcatctgttgcgggataatggcgcctgcgtgcgcagctgtccccaggacaagatggacaaggggggcgagtgtgtgccctgcaatggaccgtgccccaaaacctgcccgggcgttactgtcctgcatgccggcaacattgactcgttccggaattgtacggtgatcgatggcaacattcgcattttggatcagaccttctcgggcttccaggatgtctatgccaactacacgatgggaccacgatacataccgctggatcccgagcgacgggaggtgttctccacggtgaaggagatcaccgggtatctgaatatcgagggaacccacccgcagttccggaatctgtcgtactttcgcaatctggaaacaattcatggccgccagctgatggagagcatgtttgccgctttggcgatcgttaagtcatccctgtacagcctggagatgcgcaatctgaagcagattagttccggcagtgtggtcatccagcataatagagacctctgctacgtaagcaatatccgttggccggccattcagaaggagcccgaacagaaggtgtgggtcaacgagaatctcagggcggatctatgcgagaaaaatggaaccatttgctcggatcagtgcaacgaggacggctgctggggagctggcacggatcagtgccttacctgcaagaacttcaatttcaatggcacctgcatcgccgactgtggttatatatccaatgcctacaagtttgacaatagaacgtgcaagatatgccatccagagtgccggacttgcaatggagctggagcagatcactgccaggagtgcgtccatgtgagggacggtcagcactgtgtgtccgagtgcccgaagaacaagtacaacgatcgtggtgtctgccgagagtgccacgccacctgcgatggatgcactgggcccaaggacaccatcggcattggagcgtgtacgacgtgcaatttggccattatcaacaatgacgccacagtaaaacgctgcctgctgaaggacgacaagtgccccgatgggtatttctgggagtatgtgcatccgcaagagcagggatcgctgaagccattggccggcagagcagtttgccgaaagtgccatcccctttgcgagctgtgcacgaactacggataccatgaacaggtgtgctccaagtgcacccactacaagcgacgggagcagtgcgagaccgagtgtccggccgatcactacacggatgaggagcagcgcgagtgcttccagcgccacccggaatgcaatggttgcacgggtccgggtgccgacgattgcaagtcttgccgcaactttaagttgttcgacgcgaatgagacgggtccctatgtgaactccacgatgttcaattgcacctcgaagtgtcccttggagatgcgacatgtgaactatcagtacacggccattggaccctactgcgcagctagtccgccgaggagcagcaagataactgccaatctggatgtgaacatgatcttcattatcactggtgctgttctggtgccgacgatctgcatcctctgcgtggtcacatacatttgtcggcaaaagcaaaaggccaagaaggaaacagtcaagatgaccatggctctgtctggctgcgaggattccgagccgctgcgtccctcgaacattggagccaacctatgcaagttgcgcattgtcaaggacgccgagttgcgcaagggcggagtccttggaatgggagcctttggacgagtgtacaagggcgtttgggtgccggagggtgagaacgtcaagattccagtggccattaaggagctgctcaagtccacaggcgccgagtcaagcgaagagttcctccgcgaagcctacatcatggcctctgaggagcacgttaatctgctgaagctcctggccgtgtgcatgtcctcacaaatgatgctaatcacgcaactgatgccgcttggctgcctgttggactatgtgcgaaataaccgggacaagatcggctctaaggctctgctcaactggagcacgcaaatcgccaagggcatgtcgtatctggaggagaagcgactggtccacagagacttggctgcccgcaatgtcctggtgcagactccctcgctggtgaagatcaccgactttgggctggccaagttgctgagcagcgattccaatgagtacaaggctgctggcggcaagatgcccatcaagtggttggcactggagtgcatccgcaatcgtgtattcaccagcaagtccgatgtctgggcctttggtgtgaccatttgggaactgctgacctttggccagcgtccacacgagaacatcccagctaaggatattcccgatcttattgaagtcggtctgaagctggagcagccggagatttgttcgctggacatttactgtacactgctctcgtgctggcacttggatgccgccatgcgtccaaccttcaagcagctgactacggtctttgctgagttcgccagagatccgggtcgctatctggccattcccggggataagttcacccggctgccggcttacacgagtcaggatgagaaggatctcatccgaaaattggctcccaccaccgatgggtccgaagccattgcgaaacccgatgactacctgcaacccaaggcagcacctggtcctagtcacagaaccgactgcacggatgagatgcccaagctgaaccgctactgcaaggatcctagtaacaagaattcgagtaccggagacgatgagagggattcgagtgcccgggaagtgggcgtgggtaatctgcgcctcgatctaccagtcgatgaggatgattatctgatgcccacttgccaaccgggtcccaacaacaacaacaacatgaataatcccaatcaaaacaatatggcagctgtgggcgtggctgccggctacatggatctcatcggagtgcccgttagtgtggacaatccggagtatctgctaaacgcgcagacactgggagttggggagtcgccgatacccacccagaccatcgggataccggtgatgggaggcccgggcaccatggaggtcaaggtgccaatgccaggcagtgagccaaccagctccgatcacgagtactacaatgatacccaacgggagttgcagccactgcatcgaaaccgcaacacggagacgagggtgtag

SEQ ID NO: 22 - protein, 1210 aa, Drosophila melanogaster
MMIISMWMSISRGLWDSSSILSVLLILACMASITTSSSVSNAGYVDNGNMKVCIGTKSRLSVPSNKEHHYRNLRDRYTNCTYVDGNLKLTWLPNENLDLSFLDNIREVTGYILISHVDVKKVVFPKLQIIRGRTLFSLSVEEEKYALFVTYSKMYTLEIPDLRDVLNGQVGFHNNYNLCHMRTIQWSEIVSNGTDAYYNYDFTAPERECPKCHESCTHGCWGEGPKNCQKFSKLTCSPQCAGGRCYGPKPRECCHLFCAGGCTGPTQKDCIACKNFFDEAVSKEECPPMRKYNPTTYVLETNPEGKYAYGATCVKECPGHLLRDNGACVRSCPQDKMDKGGECVPCNGPCPKTCPGVTVLHAGNIDSFRNCTVIDGNIRILDQTFSGFQDVYANYTMGPRYIPLDPERREVFSTVKEITGYLNIEGTHPQFRNLSYFRNLETIHGRQLMESMFAALAIVKSSLYSLEMRNLKQISSGSVVIQHNRDLCYVSNIRWPAIQKEPEQKVWVNENLRADLCEKNGTICSDQCNEDGCWGAGTDQCLTCKNFNFNGTCIADCGYISNAYKFDNRTCKICHPECRTCNGAGADHCQECVHVRDGQHCVSECPKNKYNDRGVCRECHATCDGCTGPKDTIGIGACTTCNLAIINNDATVKRCLLKDDKCPDGYFWEYVHPQEQGSLKPLAGRAVCRKCHPLCELCTNYGYHEQVCSKCTHYKRREQCETECPADHYTDEEQRECFQRHPECNGCTGPGADDCKSCRNFKLFDANETGPYVNSTMFNCTSKCPLEMRHVNYQYTAIGPYCAASPPRSSKITANLDVNMIFIITGAVLVPTICILCVVTYICRQKQKAKKETVKMTMALSGCEDSEPLRPSNIGANLCKLRIVKDAELRKGGVLGMGAFGRVYKGVWVPEGENVKIPVAIKELLKSTGAESSEEFLREAYIMASEEHVNLLKLLAVCMSSQMMLITQLMPLGCLLDYVRNNRDKIGSKALLNWSTQIAKGMSYLEEKRLVHRDLAARNVLVQTPSLVKITDFGLAKLLSSDSNEYKAAGGKMPIKWLALECIRNRVFTSKSDVWAFGVTIWELLTFGQRPHENIPAKDIPDLIEVGLKLEQPEICSLDIYCTLLSCWHLDAAMRPTFKQLTTVFAEFARDPGRYLAIPGDKFTRLPAYTSQDEKDLIRKLAPTTDGSEAIAKPDDYLQPKAAPGPSHRT

SEQ ID NO: 23 - DNA, 4281 bp, Drosophila melanogaster
atgctgctgcgacggcgcaacggcccctgccccttccccctgctgcttctgctcctggcccactgcatttgcatttggcccgcgtcggcggcccgcgatcgctacgcccgccagaacaatcgccagcgccatcaggatatagatcgcgatcgggatcgagatcgattcctataccgcagcagttcggcccaaaatcgacagaggggcggggccaacttcgccctgggactgggagccaacggagtcaccattcccaccagtctggaggataagaacaagaacgagttcgtcaaggggaaaatctgcatcggcactaaatctcggctctccgtgccctccaacaaggaacatcattaccgaaacctcagagatcggtacacgaactgtacgtatgtggatggcaacttgaaactgacctggctacccaacgagaatttggacctcagcttcctagacaacatacgggaggtcaccggctatattctgatcagtcatgtggacgttaagaaagtggtgtttcccaaactacaaatcattcgcggacgcacgctgttcagcttatccgtggaggaggagaagtatgccttgttcgtcacttattccaaaatgtacacgctggagattcccgatctacgcgatgtcttaaatggccaagtgggcttccacaacaactacaatctctgccacatgcgaacgatccagtggtcggagattgtatccaacggcacggatgcatactacaactacgactttactgctccggagcgcgagtgtcccaagtgccacgagagctgcacgcacggatgttggggcgagggtcccaagaattgccagaagttcagcaagctcacctgctcgccacagtgtgccggaggtcgttgctatggaccaaagccgcgggagtgttgtcacctcttctgcgccggaggatgcactggtcccacgcaaaaggattgcatcgcctgcaagaacttcttcgacgaggcagtatcaaaggaggaatgcccgcccatgcgcaagtacaatcccaccacctatgttcttgaaacgaatcctgagggaaagtatgcctatggtgccacctgcgtcaaggagtgtcccggtcatctgttgcgggataatggcgcctgcgtgcgcagctgtccccaggacaagatggacaaggggggcgagtgtgtgccctgcaatggaccgtgccccaaaacctgcccgggcgttactgtcctgcatgccggcaacattgactcgttccggaattgtacggtgatcgatggcaacattcgcattttggatcagaccttctcgggcttccaggatgtctatgccaactacacgatgggaccacgatacataccgctggatcccgagcgacgggaggtgttctccacggtgaaggagatcaccgggtatctgaatatcgagggaacccacccgcagttccggaatctgtcgtactttcgcaatctggaaacaattcatggccgccagctgatggagagcatgtttgccgctttggcgatcgttaagtcatccctgtacagcctggagatgcgcaatctgaagcagattagttccggcagtgtggtcatccagcataatagagacctctgctacgtaagcaatatccgttggccggccattcagaaggagcccgaacagaaggtgtgggtcaacgagaatctcagggcggatctatgcgagaaaaatggaaccatttgctcggatcagtgcaacgaggacggctgctggggagctggcacggatcagtgccttacctgcaagaacttcaatttcaatggcacctgcatcgccgactgtggttatatatccaatgcctacaagtttgacaatagaacgtgcaagatatgccatccagagtgccggacttgcaatggagctggagcagatcactgccaggagtgcgtccatgtgagggacggtcagcactgtgtgtccgagtgcccgaagaacaagtacaacgatcgtggtgtctgccgagagtgccacgccacctgcgatggatgcactgggcccaaggacaccatcggcattggagcgtgtacgacgtgcaatttggccattatcaacaatgacgccacagtaaaacgctgcctgctgaaggacgacaagtgccccgatgggtatttctgggagtatgtgcatccgcaagagcagggatcgctgaagccattggccggcagagcagtttgccgaaagtgccatcccctttgcgagctgtgcacgaactacggataccatgaacaggtgtgctccaagtgcacccactacaagcgacgggagcagtgcgagaccgagtgtccggccgatcactacacggatgaggagcagcgcgagtgcttccagcgccacccggaatgcaatggttgcacgggtccgggtgccgacgattgcaagtcttgccgcaactttaagttgttcgacgcgaatgagacgggtccctatgtgaactccacgatgttcaattgcacctcgaagtgtcccttggagatgcgacatgtgaactatcagtacacggccattggaccctactgcgcagctagtccgccgaggagcagcaagataactgccaatctggatgtgaacatgatcttcattatcactggtgctgttctggtgccgacgatctgcatcctctgcgtggtcacatacatttgtcggcaaaagcaaaaggccaagaaggaaacagtcaagatgaccatggctctgtctggctgcgaggattccgagccgctgcgtccctcgaacattggagccaacctatgcaagttgcgcattgtcaaggacgccgagttgcgcaagggcggagtccttggaatgggagcctttggacgagtgtacaagggcgtttgggtgccggagggtgagaacgtcaagattccagtggccattaaggagctgctcaagtccacaggcgccgagtcaagcgaagagttcctccgcgaagcctacatcatggcctctgaggagcacgttaatctgctgaagctcctggccgtgtgcatgtcctcacaaatgatgctaatcacgcaactgatgccgcttggctgcctgttggactatgtgcgaaataaccgggacaagatcggctctaaggctctgctcaactggagcacgcaaatcgccaagggcatgtcgtatctggaggagaagcgactggtccacagagacttggctgcccgcaatgtcctggtgcagactccctcgctggtgaagatcaccgactttgggctggccaagttgctgagcagcgattccaatgagtacaaggctgctggcggcaagatgcccatcaagtggttggcactggagtgcatccgcaatcgtgtattcaccagcaagtccgatgtctgggcctttggtgtgaccatttgggaactgctgacctttggccagcgtccacacgagaacatcccagctaaggatattcccgatcttattgaagtcggtctgaagctggagcagccggagatttgttcgctggacatttactgtacactgctctcgtgctggcacttggatgccgccatgcgtccaaccttcaagcagctgactacggtctttgctgagttcgccagagatccgggtcgctatctggccattcccggggataagttcacccggctgccggcttacacgagtcaggatgagaaggatctcatccgaaaattggctcccaccaccgatgggtccgaagccattgcgaaacccgatgactacctgcaacccaaggcagcacctggtcctagtcacagaaccgactgcacggatgagatgcccaagctgaaccgctactgcaaggatcctagtaacaagaattcgagtaccggagacgatgagagggattcgagtgcccgggaagtgggcgtgggtaatctgcgcctcgatctaccagtcgatgaggatgattatctgatgcccacttgccaaccgggtcccaacaacaacaacaacatgaataatcccaatcaaaacaatatggcagctgtgggcgtggctgccggctacatggatctcatcggagtgcccgttagtgtggacaatccggagtatctgctaaacgcgcagacactgggagttggggagtcgccgatacccacccagaccatcgggataccggtgatgggaggcccgggcaccatggaggtcaaggtgccaatgccaggcagtgagccaaccagctccgatcacgagtactacaatgatacccaacgggagttgcagccactgcatcgaaaccgcaacacggagacgagggtgtag

SEQ ID NO: 24 - DNA, 4281 bp, Rattus norvegicus; identical to SEQ ID NO: 23
  CDS (1)..(3627)

SEQ ID NO: 25 - protein, 1209 aa, Rattus norvegicus
MLLRRRNGPCPFPLLLLLLAHCICIWPASAARDRYARQNNRQRHQDIDRDRDRDRFLYRSSSAQNRQRGGANFALGLGANGVTIPTSLEDKNKNEFVKGKICIGTKSRLSVPSNKEHHYRNLRDRYTNCTYVDGNLKLTWLPNENLDLSFLDNIREVTGYILISHVDVKKVVFPKLQIIRGRTLFSLSVEEEKYALFVTYSKMYTLEIPDLRDVLNGQVGFHNNYNLCHMRTIQWSEIVSNGTDAYYNYDFTAPERECPKCHESCTHGCWGEGPKNCQKFSKLTCSPQCAGGRCYGPKPRECCHLFCAGGCTGPTQKDCIACKNFFDEAVSKEECPPMRKYNPTTYVLETNPEGKYAYGATCVKECPGHLLRDNGACVRSCPQDKMDKGGECVPCNGPCPKTCPGVTVLHAGNIDSFRNCTVIDGNIRILDQTFSGFQDVYANYTMGPRYIPLDPERREVFSTVKEITGYLNIEGTHPQFRNLSYFRNLETIHGRQLMESMFAALAIVKSSLYSLEMRNLKQISSGSVVIQHNRDLCYVSNIRWPAIQKEPEQKVWVNENLRADLCEKNGTICSDQCNEDGCWGAGTDQCLTCKNFNFNGTCIADCGYISNAYKFDNRTCKICHPECRTCNGAGADHCQECVHVRDGQHCVSECPKNKYNDRGVCRECHATCDGCTGPKDTIGIGACTTCNLAIINNDATVKRCLLKDDKCPDGYFWEYVHPQEQGSLKPLAGRAVCRKCHPLCELCTNYGYHEQVCSKCTHYKRREQCETECPADHYTDEEQRECFQRHPECNGCTGPGADDCKSCRNFKLFDANETGPYVNSTMFNCTSKCPLEMRHVNYQYTAIGPYCAASPPRSSKITANLDVNMIFIITGAVLVPTICILCVVTYICRQKQKAKKETVKMTMALSGCEDSEPLRPSNIGANLCKLRIVKDAELRKGGVLGMGAFGRVYKGVWVPEGENVKIPVAIKELLKSTGAESSEEFLREAYIMASEEHVNLLKLLAVCMSSQMMLITQLMPLGCLLDYVRNNRDKIGSKALLNWSTQIAKGMSYLEEKRLVHRDLAARNVLVQTPSLVKITDFGLAKLLSSDSNEYKAAGGKMPIKWLALECIRNRVFTSKSDVWAFGVTIWELLTFGQRPHENIPAKDIPDLIEVGLKLEQPEICSLDIYCTLLSCWHLDAAMRPTFKQLTTVFAEFARDPGRYLAIPG

SEQ ID NO: 26 - DNA, 3576 bp, Danio rerio
  CDS (1)..(3573)
atggcaggaccaactgaaatcggattgttttttaccttattgctctccgggagcttctgcgcgacgccggaaaagaaagtgtgtcagggagcaaacaacaaactgactcttctgggaacggtggaagaccattatcaggttctgctcagaatgtacagaaactgcactgtggttctggagaacctggaaattacacatataacagagaaatatgacctgtccttcctcaagagcatccaggaagttggtggctatgttcttatcgcggtcaatacggtttccaaaatccctctggagaacctgcgcatcattcgcggacactcactttatgaagacaaatttgccttggccgtcctggtgaacttcaacaacagcatcgaacaaggcgtaaaagagttgccgctgactagtttaactgaaatacttaagggtggagtcaagttttgcaggaacgattatttatgtaatgtggggaccatcgagtgggccgacatcctgaacatgaagagcctgcctacaatcgtgagccataatataagctatggaaaaaactgtggaaagtgcgatccaagctgtttcaatggctcctgctggggcaccggacccgacaagtgccagagaatgacgaaagtgatctgtgcggagcagtgttcagggaggtgtaaaggacccagacccattgactgctgtaatgaacactgtgctgctggatgcactggacccagacctacagactgtctggcctgtaaggacttccaggatgaagggacatgtaaggacgcatgtccgcggctcatgctctacgacccaaacacacaccagctcgcgccaaacccatatgggaagtacagctttggggcaacgtgcatcaagacatgcccacacaactatgtggtgacggatcacggggcctgtgtgagaacatgcagccctggcacctatgaagtggatgagggtggagttcgcaaatgtaagaggtgtgaaggcctgtgtccaaaagtgtgcaatgggttgggaatggggcctttagccaatgtcctgtcaatcaatgccaccaacatcgactcctttgagaactgcactaaaatcagcggcaatgttgccatcctcagcaccacattcagaggtgacccacatactaacacttcaggactggatccagcaaagctcagtgtattgagtactgtcaaagaaatcactggttacctgatgattcagctgtggccggagagcatgcagtcccttagtgccttcgaaaaccttgaggtcatccgaggacggacaaaaacacaaggaacgtacagctttgctgtcaccaagacggccatcactcatttaggcatgcgttctctgagggagatcagtgacggggacgtgtccatcgttaagaataagaatctctgctacagcagccctgaacactggaaacgcctcttcaagtccaaacaacagtcggtcaaaatgattgaaaatatggatgctgccacctgcgccaatcagaacagcacatgtaatgagatgtgcacggctgacggctgctggggtcccggccccaccatgtgcttcggctgtgagcattacagccgcggaaaacactgcgtggcttcctgcaacctgctgaatggtgagccgcgtgaatatgaggtcaataaaacatgcatggaatgcgatcctgaatgtctgctcatgaatgaaacccagacctgcaacggccctggacccgacaaatgtacagtgtgtgcaaactataaagacggaccgcactgtgtgcatcgctgcccgcaaggtgtaccaggagagaaagacacactcatctggaaatacgctgacgtgacacacgtttgccagccctgtcatgaaaactgcacccagggatgtacggggcctgatctaaaggactgcaaagatttcaaaagctctggtttgccgatgatcgctgctggcgttgtcggaggtctactggcgtttgttattctggctcttggagtggccgttctcctgcgcagacgccacatccggaggaagaggactctgagacgactcctgcaagagagagagcttgtggagcctctgacccccagcggcgaagcccccaatcaggccttactgcgcatcctcaaagagacggagttcaagaagatcaaagtgctgggctccggggctttcggcactgtgcacaagggcctttgggttccagaaggagagaatgtgaagatccctgtcgccatcaaggtcttaagagaagccacgtctcccaaagctaacaaggagataatggatgaggcgtacgtcatggccagtgttgagcatccccatgtgtgtcgtctgctgggcatctgcttgacctccacagtgcagctcatcactcagctgatgccctacggctgcttgctggactatgtcagagaaaacaaggaccgcatcggctcccagcacctgctcaactggtgcgtgcagatcgctaaaggtatgaattatctagaagagcgccatcttgtgcaccgagacctggcagcacgtaatgtgttggtaaagacgcctcagcatgtcaagattaccgatttcggcctcgccaagctgttaaacgcggacgagaaggagtatcacgcagatggaggaaaggttccaattaaatggatggcgctggagtccatccagcacaggacttacacccaccagagtgacgtctggagctacggtgtgaccgtctgggagttgatgacgtttgggacgaaaccttatgatgggattcctgccagtgaaatcgccggagttctggaaaaaggagaaagacttcctcaaccccccatctgcaccattgatgtgtacatgatcatggtcaaatgttggatgattgatgctgagagcagaccccgcttcagggagctcatcgcagagttcactaaaatggctcgtgacccgtcccgctatctggtcattcagggggacgaccgcatgcatttacccagtccttctgactccaagttctaccgcagcctgatgagcggagagctggacgaggccgtggacgcagacgagtatttagtgcccaatcacagcttcttcagcagcccgagcacgtcccgcacacaactgctgcactctgtgagcctgaacagcagctttggaaactgtaatagtagaaacgggaatggttatccagtgagggagaacagcatggtcctgcgctacatcccagaccccacagagcgctttcaggagggagactttcagcctgcgccgggttataacgaatatatgaaccagaatgagtccagcatgatcaacccagtgtaccagcagccccacggacccccgcggaccctcctccactcctccccagcgctggacgagacggaagaggagtatctgaactgcttcaagagcccggctccggcttcagtggtggagtatctgaacacgtcccacacacagctgctctccacaaagcccttcttcagcatggacaaccccgactaccagcaggacttctgcccgctggagctcaaaacacacaccaacgggcacctgccggccgcgcagaaccaggagtacatgggcctggaggtgcactag

SEQ ID NO: 27 - protein, 1191 aa, Danio rerio
MAGPTEIGLFFTLLLSGSFCATPEKKVCQGANNKLTLLGTVEDHYQVLLRMYRNCTVVLENLEITHITEKYDLSFLKSIQEVGGYVLIAVNTVSKIPLENLRIIRGHSLYEDKFALAVLVNFNNSIEQGVKELPLTSLTEILKGGVKFCRNDYLCNVGTIEWADILNMKSLPTIVSHNISYGKNCGKCDPSCFNGSCWGTGPDKCQRMTKVICAEQCSGRCKGPRPIDCCNEHCAAGCTGPRPTDCLACKDFQDEGTCKDACPRLMLYDPNTHQLAPNPYGKYSFGATCIKTCPHNYVVTDHGACVRTCSPGTYEVDEGGVRKCKRCEGLCPKVCNGLGMGPLANVLSINATNIDSFENCTKISGNVAILSTTFRGDPHTNTSGLDPAKLSVLSTVKEITGYLMIQLWPESMQSLSAFENLEVIRGRTKTQGTYSFAVTKTAITHLGMRSLREISDGDVSIVKNKNLCYSSPEHWKRLFKSKQQSVKMIENMDAATCANQNSTCNEMCTADGCWGPGPTMCFGCEHYSRGKHCVASCNLLNGEPREYEVNKTCMECDPECLLMNETQTCNGPGPDKCTVCANYKDGPHCVHRCPQGVPGEKDTLIWKYADVTHVCQPCHENCTQGCTGPDLKDCKDFKSSGLPMIAAGVVGGLLAFVILALGVAVLLRRRHIRRKRTLRRLLQERELVEPLTPSGEAPNQALLRILKETEFKKIKVLGSGAFGTVHKGLWVPEGENVKIPVAIKVLREATSPKANKEIMDEAYVMASVEHPHVCRLLGICLTSTVQLITQLMPYGCLLDYVRENKDRIGSQHLLNWCVQIAKGMNYLEERHLVHRDLAARNVLVKTPQHVKITDFGLAKLLNADEKEYHADGGKVPIKWMALESIQHRTYTHQSDVWSYGVTVWELMTFGTKPYDGIPASEIAGVLEKGERLPQPPICTIDVYMIMVKCWMIDAESRPRFRELIAEFTKMARDPSRYLVIQGDDRMHLPSPSDSKFYRSLMSGELDEAVDADEYLVPNHSFFSSPSTSRTQLLHSVSLNSSFGNCNSRNGNGYPVRENSMVLRYIPDPTERFQEGDFQPAPGYNEYMNQNESSMINPVYQQPHGPPRTLLHSSPALDETEEEYLNCFKSPAPASVVEYLNTSHTQLLSTKPFFSMDNPDYQQDFCPLELKTHTNGHLPAAQNQEYMGLEVH

(19) United States
(12) Patent Application Publication
(10) Pub. No.: US 2007/0275049 A1
(43) Pub. Date: Nov. 29, 2007

(54) Anti-tumor molecular vacine and method of making thereof

(75) Inventors: Yuquan Wei, Chengdu (CN); Ling Tian, Chengdu (CN)

Correspondence Address: BEN WANG, 706 COLORADO AVE, PALO ALTO, CA, 94303, US

(73) Assignee: Shen Zhen Tsinghua Yuanxing Bio-Pharm Science & Technology Co., Ltd., Shenzhen (CN)

(21) Appl. No.: 11/649,155

(22) Filed: Dec. 27, 2006

Related U.S. Application Data
(63) Continuation of application No. 10/749,104, filed on Dec. 29, 2003, now abandoned.

(30) Foreign Application Priority Data
Dec. 25, 2003 (CN) 200310113054.0
Dec. 27, 2002 (CN) 02159602

(51) Int. Cl. A61K 39/00; A61K 39/02; A61K 39/12; A61K 39/23; A61K 9/127; A61K 9/14; C12N 5/00; G01N 33/574

(52) U.S. Cl. 424/450; 424/184.1; 424/200.1; 424/204.1; 424/233.1; 424/277.1; 424/489; 435/252.3; 435/325; 435/375; 435/7.23

(57) ABSTRACT

The present invention provides a vaccine which is capable of inhibiting the growth and formation of tumors which can express an endogenous tumor specific protein. Also disclosed are methods of administering the vaccine to a subject bearing a tumor whose formation and growth is inhibited by the vaccine.

### CROSS-REFERENCE TO RELATED APPLICATIONS

This application is a continuation application, and claims the benefit of U.S. nonprovisional application Ser. No.: 10/749,104, filed on Dec. 29, 2003, which claims the benefit of Chinese patent application 200310113054.0, filed on Dec. 25, 2003, and Chinese patent application Serial No.: Serial No.: 02159602, filed on Dec. 27, 2002, the contents of which are hereby incorporated by reference in its entirety into this application.

### FIELD OF THE INVENTION

The present invention relates to a novel anti-tumor vaccine, including anti-EFGR molecular vaccine. The field of molecular vaccine encompasses many aspects of biotechnology, such as PCR, Molecular Cloning, Gene Expression, Modem vaccine technology, and biotechnological medicine and pharmacology.

### BACKGROUND

Vaccines are materials that are capable of stimulating the immune system to initiate an immune response against specific target substances such as virus or bacteria. Classical concept of vaccine originated from the process of generating immunity against infectious diseases. That process mainly consisted of using treated microbial pathogen (such as virus and bacteria) or its derivatives to immunize the organism so as to produce humoral immune response to prevent the onset of infectious diseases. For example, inactivated vaccine is derived by inactivating the active component of the infectious pathogen. Attenuated vaccine is produced by mutating live virus or bacteria so that it cannot reproduce itself in biological organism. These two classes of vaccines achieve the goal of immunization through the surface antigen of the pathogen and their reaction with B cells and T cells.

Cancer vaccines are of particular importance in cancer therapy. This type of vaccine is different from traditional, preventive vaccine, in that, it is mainly used on patients who are already inflicted with cancer. The purpose of using cancer vaccine is to stimulate the patient's specific immune response against the cancer, to the point that eventually the cancer is effectively rejected by the organism, and therefore cured. The research and development of cancer vaccine has become a focal point of cancer therapy internationally. Cancer vaccines mainly include cancer cell vaccine, gene-modified vaccine, polypeptide tumor vaccine, and gene/DNA vaccine, etc.

Cancer cell vaccines are intact, dead cells produced by treating cancer cells of a patient or animal with physical or chemical methods, such that after treatment, those cells will possess therapeutic or auxiliary therapeutic effect. The methods used to treat the cancer cells include X-ray radiation, or treating with organic solvent, etc. After introducing this vaccine to the patient using injection, or other method, the cancer cell vaccine can stimulate or enhance the patient's immune response against the targeted cancer. Genetically-modified vaccine, polypeptide cancer vaccine, and gene/DNA vaccines are all vaccines having therapeutic effect on the targeted cancer, and made by using the cancer antigen or its fragments, or polynucleotides coding for such cancer antigen or its fragments, and carriers/cells containing the polynucleotides.

Cancer vaccine studies have become an important area in the fight to cure cancer and save the lives of thousands of patients worldwide. It has been recognized in the medical research and clinical studies that one of the key factor for the success of any cancer therapy is its ability to distinguish neoplastic cells, which should be killed by the therapy, from normal cells, which should be unaffected, and left alone as much as possible, by the therapy. In reality, though, it is also difficult, if not impossible, to achieve this goal. Many available cancer therapy protocols are based upon the differential growth rate of cancer cells, that is to say, cancer cells tend to divide and proliferate much faster than normal cells. But, such cancer therapies would not be effective in the case of cancers such as brain tumor. An ideal situation is to use a tumor specific antigen which is exclusively expressed on tumor cells to immunize the patient or subject animal, because the immune system would most efficiently recognize an antigen that is has never seen before. In that case, it would be possible to use one's own immune system to destroy a cancer. Under this reasoning, the success rate of the approach would depend upon the identification of an appropriate antigen that can elicit a humoral or cellular immune response against the targeted tumor.

Research developed over the years in the past has uncovered many tumor specific proteins and molecules that are specifically related to the onset of a particular cancer. These molecules are generally referred herein as tumor-specific proteins. Many of the TSPs are good candidates for tumor antigen. One of such TSP is epidermal growth factor receptor, EGFR. EGFR is a trans-membrane, single chain, glycosylated protein with a molecular weight of 170 KD. It is consisted of 1186 amino acid residues, and possesses Tyrosine Protein Kinase (PTK) activity. The structure of the EGFR molecule is consisted of three main parts: an extracellular domains a transmembrane domain, and an intracellular domain. The ligand binding activity occurs in the extracellular domain and the PTK activity resides within the intracellular domain. Its ligand, EGF, or TGF-α can act on EFGR through either autocrine pathway, or paracrine pathway, by activating PTK, and through a series of signal transduction, causing cells to divide and proliferate. EFGR can be widely found on the surface of normal mammalian epithelial cell surface. On average, there are about 50-100 thousand receptor molecules per cell. Many cancer cells such as lung cancer, breast cancer, ovary cancer, colon cancer, prostate cancer, bladder cancer, head and neck squamocarcinoma and glioma, all have over-expressed EGFR, up to 1-3×10⁶ molecules. Take, as an example, in the case of lung cancer, the overexpression of EGFR is closely related to the cancer's infiltration, metastasis and prognosis. Hence, EGFR has been commonly accepted as a tumor specific antigen. Logically, EGFR has been hailed as one of the ideal target molecules in cancer therapy. So far, however, the major application of using EGFR as a therapy target has been with monoclonal antibody and small molecular synthetic compounds. There has been some positive development in areas such as using EGFRvIII molecule in tumor peptide vaccine, and anti-sense RNA gene therapy. Nonetheless, there have been few reports in literature regarding using EGFR as a target molecule in making anti-tumor molecular vaccine. Thus, there exists a need to develop new vaccines to specifically target tumor antigens such as EGFR in order to inhibit the growth and formation of tumors.

### SUMMARY OF THE INVENTION

The present invention provides anti-tumor tumor specific protein (TSP) molecular vaccine, including autologous TSP varian vaccine, xenogeneic TSP vaccine, and gene directed evolution TSP vaccine. As a preferred embodiment, such TSP is a tumor receptor protein. A further preferred embodiement of the TSP molecular vaccine is the Epidermal Growth Factor Receptor (EFGR) molecular vaccine. TSP molecular vaccines encompass a variety of biological vaccine and they can be made using as antigen a xenogeneic homologous TSP molecule that is either genetically engineered, mutated, and improved, or, derived from human or other different species due to natural evolution. TSP molecular vaccine includes recombinant protein vaccine, recombinant gene vaccine, recombinant viral vaccine, modified gene vaccine, and stable transformants of commensal bacteria. Tumor receptor vaccine is a new type of tumor vaccine.

An object of the present invention is to provide a vaccine which comprises a molecular homolog having sufficient structural similarity to a tumor specific protein endogenously expressed in a tumor such that the molecular homolog is capable of inducing an immune response to the tumor specific protein in a subject bearing the tumor.

Another object of the invention is to provide a method for inducing immunity against a tumor specific protein endogenously expressed in a tumor which comprises administering to a subject bearing the tumor a molecular homolog having sufficient structural similarity to the tumor specific protein so as to enable the molecular homolog to induce the immunity against the tumor specific protein.

A further object of the invention is to provide a method of immunizing an animal, preferably a human, against a tumor having a tumor specific protein endogenously expressed therein, comprising the step of administering to the animal a molecular homolog having sufficient structural similarity to the tumor specific protein so as to enable the molecular homolog to induce an immune response to the tumor specific protein.

Yet another object of the invention is to provide a method of making a vaccine for inducing an immune response against a tumor specific protein endogenously expressed in a tumor, which comprises selecting a molecular homolog having sufficient structural similarity to the tumor specific protein so as to enable the molecular homolog to induce the immune response against the tumor specific protein in the subject bearing the tumor.

### BRIEF DESCRIPTION OF THE FIGURES

FIG. 1 is a schematic diagram of the mechanism of action of homologous molecular vaccine.

FIG. 2 shows recombinant EGFR plasmid maps. 2A shows recombinant EGFR eukaryotic expression plasmid maps; 2B shows recombinant EGFR prokaryotic expression plasmid maps; 2C shows recombinant EGFR yeast expression plasmid maps; 2D shows recombinant EGFR Adenovirus shuttle plasmid maps; 2E shows recombinant EGFR Lentivirus precursor plasmid maps.

FIG. 3 is a flow chart depicting the construction of EGFR recombinant protein vaccine.

FIG. 4 is a flow chart depicting the construction of EGFR recombinant virus vaccine.

FIG. 4A is a flow chart depicting the construction of EGFR recombinant Adenovirus vaccine; FIG. 4B is a flow chart depicting the construction of EGFR recombinant Lentivirus vaccine.

FIG. 5 is a flow chart depicting the construction of the RGD-modified EGFR recombinant Adenovirus vaccine.

FIG. 6, including FIGS. 6A, 6B and 6C, is a schematic diagram depicting the mechanism of action for the nanoparticle targeted EGFR molecular vaccine.

FIG. 7 is a graph showing the induction of protective anti-tumor immunity of EGFR recombinant DNA vaccine. 7A. Tumor Volume changes of immunized mice bearing LL/2c Lewis lung cancer. 7B. Tumor Volume changes of immunized mice bearing MA782/5S mammary cancer. 7C. Survival rate of immunized mice bearing LL/2c Lewis lung cancer. 7D. Survival rate of immunized mice bearing MA782/5S mammary cancer. hEe-p, human EGFR extracellular DNA vaccine; mEe-p, mouse EGFR extracelluar DNA vaccine; c-p, blank plasmid control; Saline, saline control.

FIG. 8 is a graph showing the induction of therapeutic anti-tumor immunity of EGFR recombinant DNA vaccine. 8A. Tumor Volume changes of immunized mice bearing LL/2c Lewis lung cancer. 8B. Tumor Volume changes of immunized mice bearing MA782/5S mammary cancer. 8C. Survival rate of immunized mice bearing LL/2c Lewis lung cancer. 8D. Survival rate of immunized mice bearing MA782/5S mammary cancer. hEe-p, human EGFR extracellular DNA vaccine; mEe-p, mouse EGFR extracelluar DNA vaccine; c-p, blank plasmid control; Saline, saline control.

FIG. 9 depicts the induction of anti-tumor immunity of EGFR recombinant protein vaccine. 9A. Protective immunity. 9B. Therapeutic immunity. 9C. Survival curves for mice bearing MA782/5S mammary cancer. edCER: Recombinant protein vaccine containing Chicken EGFR extracellular domain. edMER: Recombinant protein vaccine containing Mouse EGFR extracellular domain. Adj: Adjuvant. NS: Saline.

FIG. 10 depicts the anti-tumor metastasis effect of EGFR recombinant protein vaccine. 10A. Number of transferer of LL/2c lung cancer. 10B. Lung wet weight after LL/2c tumor metastasis. edCER: Recombinant protein vaccine containing Chicken EGFR extracellular domain. edMER: Recombinant protein vaccine containing Mouse EGFR extracellular domain. Adj: Adjuvant. NS: Saline.

FIG. 11 depicts EGFR molecular vaccine's inhibition of tumor cell growth in vitro, and its adoptive anti-tumor immunity. 11A. The inhibition of growth of EGFR-positive tumor cells (A549, LL/2c, MA782/5S) and EGFR-negative tumor cells (H22 and MMT-06052) by Ig derived from mice immunized with EGFR Recombinant DNA vaccine hEe-p. 11B. Inhibition of growth of tumor cells by Ig derived from non-immunized normal mice. 11C. In vivo adoptive anti-tumor immunity of Ig derived from mice immunized with EGFR recombinant DNA vaccine hEe-p. hEe-p, human EGFR extracellular DNA vaccine; mEe-p, mouse EGFR extracelluar DNA vaccine; c-p, blank plasmid control; Saline, saline control.

FIG. 12 depicts the types of antibodies induced by EGFR molecular vaccine. 12A. Types of antibodies induced by EGFR recombinant DNA vaccine. 12B. Types of antibodies induced by EGFR recombinant protein vaccine. hEe-p, human EGFR extracellular DNA vaccine; mEe-p, mouse EGFR extracelluar DNA vaccine; c-p, blank plasmid control; Saline, saline control. edCER: Recombinant protein vaccine containing Chicken EGFR extracellular domain. edMER: Recombinant protein vaccine containing Mouse EGFR extracellular domain. Adj: Adjuvant. NS: Saline.

FIG. 13 depecits the induction of CTL-mediated cytotoxicity in vitro with EGFR DNA vaccine and adoptive transfer of T cell subsets. A, T cells derived from the spleens of hEe-p-immunized mice were tested against LL/2c cells at different E:T ratios by a standard ⁵¹Cr release assay. T cells derived from the spleens of hEe-p-immunized mice showed higher cytotoxicity against LL/2c cells than did T cells from mEe-p, c-p, or nonimmunized mice by a standard ⁵¹Cr release assay. hEe-p-induced tumor killing activity can be blocked by anti-CD8 or anti- MHC class I (anti-H-2Kb/H-2Db) mAb. B and C, T cells were isolated from spleens of C57BL/6 mice, immunized with hEe-p (□), mEe-p, and nonimmunized mice (▪), and were depleted of CD4+ or CD8+ lymphocytes. The adoptive transfer of 2×10⁷ CD4-depleted (CD8+) (B) or CD8-depleted (CD4+) (C) T cells from mice immunized with hEe-p showed the antitumor activity against EGFr positive LL/2c. D and E, The adoptive transfer of 2×10⁷ CD4-depleted (CD8+)(D) or CD8-depleted (CD4+) (E) T cells from BALB/c mice immunized with hEe-p was also found to be effective in EGFr-positive MA782/5S mammary cancer model. There was no antitumor activity found in syngeneic EGFr-negative tumor (B16 and Meth A) (B-E). In addition, the transfer of T lymphocyte subsets from mice immunized with mEe-p and nonimmunized mice (▪) had no effect (B-E). Data represent day 25 after tumor cell injection.

FIG. 14 depicts the identification of auto-antibodies on the tumor cells by fluorescence microscopy. There was the deposition of auto-antibodies on LL/2c tumor cells (A) and MA782/5S tumor cells (B) from hEe-p-immunized mice, but not on the corresponding tumor cells from non-immunized mice (C and D). The mice depleted of CD4+T lymphocytes were immunized with hEe-p and did not develop detectable IgG-specific fluorescence on LL/2c tumor cells (E) and MA782/5S tumor cells the tumor cells (F). In contrast, the depletion of CD8+ lymphocytes (G and H) or NK cells showed no effect. There was no deposition of auto-antibodies within the tissues of the liver (I) and kidney (J) of mice immunized with hEe-p and the corresponding tissues (K and L) from non-immunized mice.

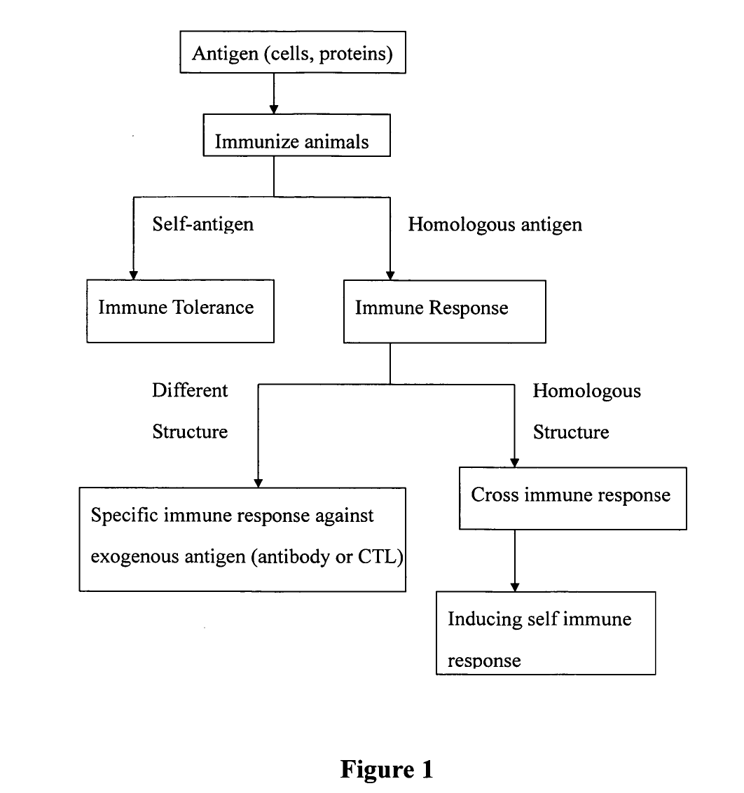

### DETAILED DESCRIPTION OF THE INVENTION

The present invention deals with a new type of anti-cancer vaccine—tumor specific protein molecular vaccine, such as tumor receptor vaccine, i.e. EGFR molecular vaccine. The description hereinafter is provided in some detail with respect to an embodiment of the invention, namely, EGFR molecular vaccine, and its use in animal tumor model. However, as discussed above, the practices of the present invention can also be applied to the production and use of other tumor-specific receptor such as VEGF, TGF-β, etc.

In the description of the invention set forth hereinabove, emphasis has been placed upon the preparation of a vaccine based upon the EGFR molecule. It is clearly indicated, however, that the concept and practices of this invention are generally applicable to the preparation of vaccines based upon other tumor associated proteins including other tumor specific growth factor receptors to treat or prevent human cancers and for the preparation of vaccines to treat or prevent infectious diseases in man and animals.

In order to confirm the hypothesis mentioned above, we selected some cancer cell proliferation-associated molecules (such as EGFR, insulin-like growth factor receptor (IGFR), etc) or tumor angiogenesis-associated molecules (such as vascular endothelial growth factor (VEGF), αvβ3 integrin, endoglin, vascular endothelial growth factor receptor (KDR), fibroblastic growth factor receptor (FGFR) and Tie2, etc., from Xenopus laevis, bird, mouse, pig, bovine, even from the fruit fly Drosophila melanogaster as target molecules. We isolated the counterparts of these homologous genes, immunized mouse models with the plasmid DNA vector or adenoviral vector inserted with these xenogeneic genes or their recombinant proteins or synthetic peptides, observed their activity of anti-tumors (including mammary cancer, lung cancer, melanoma, hepatocarcinoma and fibrosarcoma) and explored the possible molecular and immunological mechanisms.

As used herein, the term “tumor specific protein” refers to a protein which is specifically associated with a particular tumor, and is not usually found in normal cells, or if it is found in normal cells, it is uncontroallable overexpressed in the tumor, as if often the case. One group of tumor specific protein (TSP) is tumor receptor protein which, after being bound by a ligand molecule, activates a cellular mechanism to enable the tumor cells to growth uncontrollably. Examples of a tumor receptor protein include Epidermal Growth Factor Receptor, EGFR, insulin-like growth factor receptor (IGFR). Other examples of TSP include tumor angiogenesis-associated molecules such as vascular endothelial growth factor (VEGF), αvβ3 integrin, endoglin, vascular endothelial growth factor receptor (KDR), fibroblastic growth factor receptor (FGFR) and Tie2, etc.

As used herein, the term “molecular homolog” means either a DNA molecule, or, a protein or polypeptide molecule, which share sequence similarity with another corresponding molecule.

As used herein, the term “structural similarity” refers to sequences similarity between two DNA molecules, or two Protein molecules, respective. Such similarity can be deduced using industry standard tools such as BLAST and other bioinformatical software.

As used herein, the term “endogenously expressed” refers to a gene that is found to be in existence in the native cell, and not introduced through a foreign vector.

As used herein, the term “a subject” refers to an animal, including a human, mouse, a bird, a chick, nematode, etc.

As used herein, the term “an immune response” refers to a reaction by the subject to fight against a recognized antigen, the reaction can be the generation of antibody which specifically binds the antigen, or the reaction can be a cytotoxic T-Lymphocyte activity (CTL)

As used herein, the term “bearing” means that the subject carries the tumor within its organs, tissues, etc.

As used herein, the term “xenogeneic homolog” refers to a protein or a gene which is derived from an animal of different species from the molecule of similar biological function that it's compared to.

As used herein, the term “genetic engineering” refers to various techniques that can be used to generate DNA or protein homolog, such as error-prone PCR, random primer extension technique, and DNA shuffling technique, which can cause artificial mutation in autologous EGFR molecule, and then establish a gene mutation library.

1. Origin of EGFR Molecule, its Selection and Improvement

The term EGFR molecule as used herein includes modified autologous EGFR molecule, and xenogeneic EGFR molecule, and gene-directed evolution EGFR molecule. A key feature of the present invention is demonstrated in the use of xenogeneic homologous EGFR molecule as a molecular antigen in anti-tumor immunotherapy, said EGFR molecular vaccine is made either with genetic engineering, or derived from the natural evolutionary process, and the differences between the sequence of the molecular antigen and sequence of the EGFR of the target subject, such as an animal or human is utilized to illicit an immune response against the EGFR of the target subject.

EGFR is widely found in nature in a variety of organisms ranging from mammals such as human, mouse, to avian organism such birds and chickens, to lower organism such as nematode, and fruit fly. The EGFR molecules from these different organisms have certain differences amongst them. Their homology amongst each other is between 30% to 100%. The homology between the xenogeneic EGFR molecules as disclosed in the present invention is between 45% to 95%, and as examples, we used human, mice, chicken, and Drosophila as exemplary species to discuss the invention. At the amino acid level, the homology between mouse EGFR and human, chicken, and Drosophila is 87%, 72%, and 55%.

Comparative genomics pointed out that there are 70 to 100 thousands of genes in all mammals, a certain number of which are rather conserved. These genes show certain degree of similarity in structures and/or functions between different species. They are xenogeneic homologous genes. For example, epidermal growth factor receptor (EGFR) genes of human have the homology of 88%˜93% with mice, 72%˜83% with birds, and 40%˜56% with fruit flies.

One of the classic problems in using tumor receptor as antigen in immunotherapy is that autologous EGFR molecule is immuno-tolerant by the host body, and thus its immunogenicity is very weak, if at all. By using modern biotechnological engineering, autologous EGFR molecules can be modified with the procedure of gene directed evolution, and thus improved and enhance the immunogenicity of the EGFR antigen. The technical protocol is generally using error-prone PCR, random primer extension technique, and DNA shuffling technique, and cause artificial mutation in autologous EGFR molecule, and then establish a gene mutation library. After that, undertake a process of selection using phage display technique, ribosome display technique, and obtain EGFR molecules with stronger immunogenicity. In addition, we can utilize the differences between the expression modification systems of bacteria, virus and other organisms, and further modify and improve the autologous EGFR molecules at the protein level, in order to increase the immunogenicity of autologous EGFR.

It has been known that tumor cells would produce one or more kinds of tumor antigens during its course of malignant transformation and proliferation. However, in most cases, tumor antigen is differentiation antigen with very weak immunogenicity that is not enough to induce active immune response. In addition, in the view of immunology, tumor cells themselves are the cells of host, which can continuously express “normal” antigen (gene overexpression) and/or abnormal antigen (resulting from gene modification, mutation or deletion). We can therefore consider tumor antigen as self-antigen in this sense. In normal physiological state, a body cannot evoke immune response to self-antigen, which is immune tolerance. In fact, self-antigen is the most compatible and the richest antigen to which the host immune system must tolerate. The induction and maintenance of self-antigen is mediated by several kinds of mechanisms by which the normal tissues can be protected from improper injury. However, when the host cryptic antigen releases or changes by some kinds of biological, physical or chemical reasons, it can induce autologous immune response, which acts on the tissues or cells the target antigen located and results in pathological changes and blocking the function of the corresponding organelle. So, if we can release the cryptic self-antigen of tumor cells or modify it to somewhat, we could therefore induce specific autologous immune response to autologous tumor cells, and consequently tumor regression or suppression. In other words, we can take cancer immunotherapy by inducing autologous immune response of a body.

The cDNA sequence corresponding to normal EGF receptor has been reported by Ullrich et al., in Nature 1984 309, 418-425, Ullrich, A., Coussens, L., Hayflick, J. S., Dull, T. J., Gray, A., Tarn, A. W., Lee, J., Yarden, Y, Libermann, T. A., Schlessinger, J., et al. (1984). Human Epidermal Growth Factor Receptor cDNA Sequence and Aberrant Expression of the Amplified Gene in A431 Epidermoid Carcinoma Cells. Nature 309:418-425, and was characterized the genetic alterations associated with rearrangements or deletions of this gene in five malignant gliomas. EGF receptor gene is expressed on the cell surface (Humphrey et al., Cancer Research 1988, 48, 2231-2238). The EGF receptor gene has been shown to be amplified in 40% of glioblastoma multiform tumors (Libermann et al., Nature 1985, 313(5998), 144-7; Wong et al., Proc Natl Acad Sci USA 1987 84(19), 6899-903). This receptor has been implicated in a wide variety of tumors including those of the breast, skin and bladder (Harris, A. L. Recent Results in Cancer Research 1989, 113, 70-77).

It has been known that tumor cells would produce one or more types of tumor antigens during its course of malignant transformation and proliferation. However, in most cases, tumor antigen is differentiation antigen with very weak immunogenicity that is not enough to induce active immune response. In addition, in the view of immunology, tumor cells themselves are the cells of host, which can continuously express “normal” antigen (gene overexpression) and/or abnormal antigen (resulting from gene modification, mutation or deletion). We can therefore consider tumor antigen as self-antigen in this sense. In normal physiological state, a body cannot evoke immune response to self-antigen, which is immune tolerance. In fact, self-antigen is the most compatible and the richest antigen to which the host immune system must tolerate. The induction and maintenance of self-antigen is mediated by several kinds of mechanisms by which the normal tissues can be protected from improper injury. However, when the host cryptic antigen releases or changes by some kinds of biological, physical or chemical reasons, it can induce autologous immune response, which acts on the tissues or cells the target antigen located and results in pathological changes and blocking the function of the corresponding organelle. So, if we can release the cryptic self-antigen of tumor cells or modify it to somewhat, we could therefore induce specific autologous immune response to autologous tumor cells, and consequently tumor regression or suppression. In other words, we can take cancer immunotherapy by inducing autologous immune response of a body.

In general, cancer vaccines are based on the weak immunogenicity of target tumor antigen mixed with adjuvant in order to produce, recover or enhance anti-cancer immune response and kill the residual or invasive tumor cells. The potential target of anti-self-antigen or anti-tumor includes over expressed protein, tissue-specific differentiation antigen, development protein which tumor cells abnormally expressed, and so on. How to enhance the immunogenicity of target antigen then?

As we mentioned above, there exist a certain number of xenogeneic homologous genes between various species. It is commonly expected that the xenogeneic homologous genes between various species should have some immunological significance in the course of evolution. One could take advantage of the subtle differences of xenogeneic homologous genes derived from evolution to break immune tolerance, enhance immunogenicity and induce autologous immune response of tumor cells, which leading to the killing of tumor cells. The mechanism of which probably as follows: although the neutral mutation of xenogeneic homologous gene from evolution does not lose or change its function, it probable affects or changes its mode of immune response. When xenogeneic homologous genes are introduced into a host and expressed corresponding xenogeneic homologous protein, the host will recognize it as foreign antigen and eliminate it by producing the specific antibodies or CTL, on the other hand, it will lead non-specific cross immune reaction because of the similarity between xenogeneic homologous proteins and the related protein in the host, and thereby inducing autologous immune response and breaking the immune tolerance of the body to this protein. Maybe the immunological rejection during heterogenous transplantation is also due to the existence of xenogeneic homologs (such as genes, peptides or proteins).

The gene-directed evolution technology refers to the method of using artificial technique to change the characteristics of a particular gene in a significant way, for example, to change an enzyme with low catalytic activity in a reaction to enable the enzyme to become highly catalytic in the reaction. The process of gene-directed evolution speeds up the pace of changes, and thus bypassing the long and arduous process of gradual changes that the natural course of evolution takes. Techniques used in gene-directed evolution include error-prone PCR, and DNA shuffling technique, etc. For detailed description of the gene-directed evolution method, see Beaudry A A, Joyce G F. Directed evolution of an RNA enzyme. Science, 1992, 257: 641-644.

Recent development in modern biotechnology provides a variety of techniques such as error-prone PCR technology, DNA Shuffling, Phage Display technique, etc, in order to achieve TSP molecule, or EFGR molecular vaccine made with gene-directed evolution technique.

2. EGFR Recombinant Gene Vaccine

The present invention can be described at the gene level using EGFR recombinant gene vaccine, as described below. Gene vaccine, also known mainly as DNA vaccine, is a new type of vaccine based on nucleic acids made with modern molecular technology.

The EGFR molecular vaccine of the present invention include DNA vaccine. We search for sequences of EGFR collected in publicly available databanks such as GenBank (including gene sequences, cDNA sequences, mRNA sequences and amino acid sequences). Using these sequences, we designed primers or probes, and using techniques such PCR, RT-PCR, molecular hybridization, we isolated, from various commercial gene library, cDNA library, cell lines, tissue cultures, we cloned and isolated the intracellular domains of the EGFR molecules from a variety of different species. We have found that the extracellular domain of EGFR is the preferred active region causing immunogenicity. In addition, we can further use the gene directed evolution technique to select EGFR molecules with stronger immunogenicity. After confirming the sequences of the extracellular domain of the EGFR cDNAs using sequencing, we constructed Eukaryotic plasmid expression systems containing these intracellular domains of the EGFR using molecular cloning technique. These constructs are then transfected to CHO cell lines, and they are observed and tested for its EGFR expression and the level of expression. The recombinant EGFR constructs of these Eukaryotic plasmid expression systems can be analyzed and confirmed using restriction enzyme digestion, SDS-PAGE, and Western Blot. We used Alkaline Extraction to obtain confirmed recombinant EGFR expression plasmids, and then using ultracentrifuge, ultra-filtration methods to eliminate E. Coli endotoxins. After that, we get pure recombinant plasmid DNA. The plasmid DNA molecules can be used as DNA vaccine for use to immunize animals.

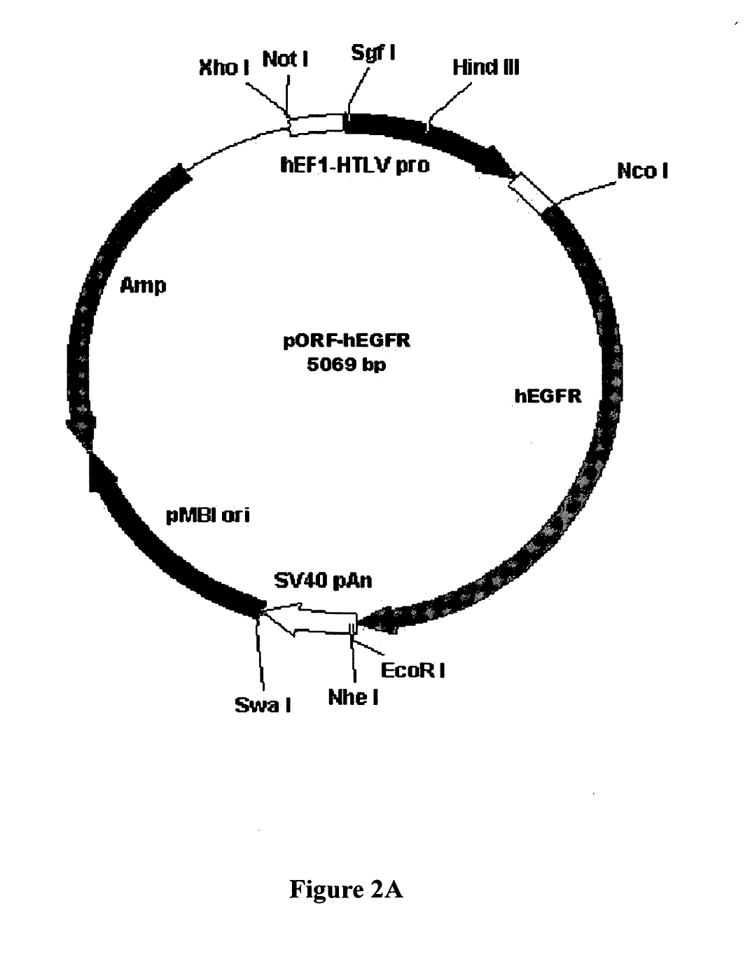

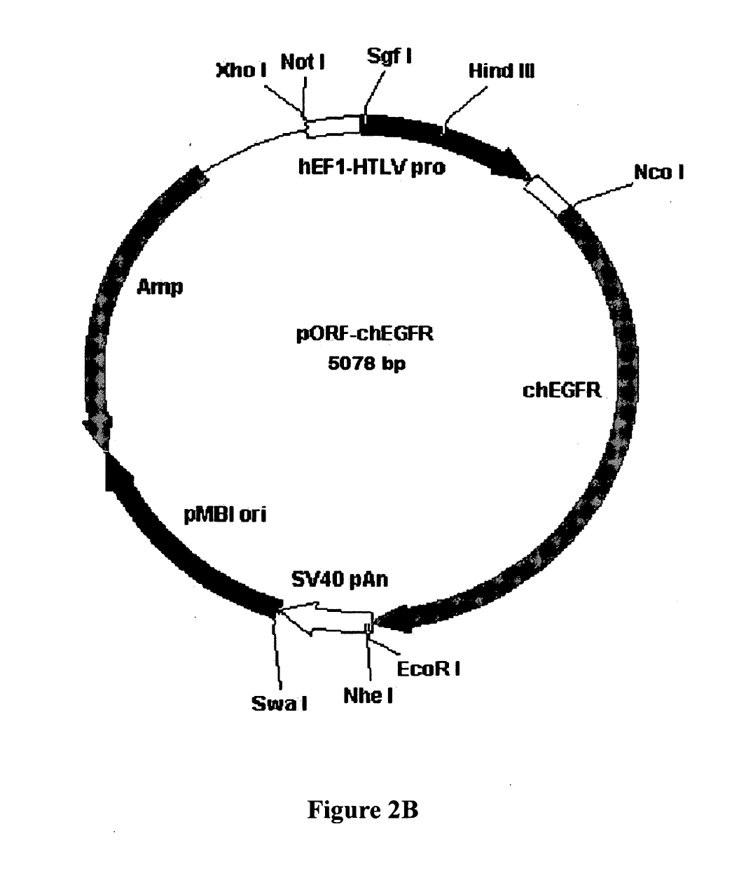

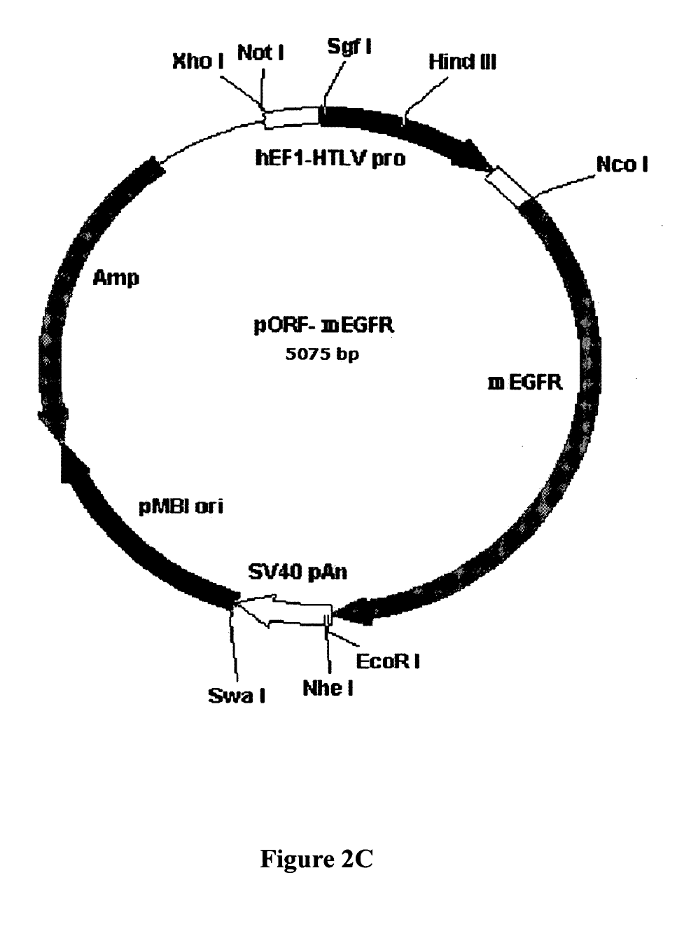

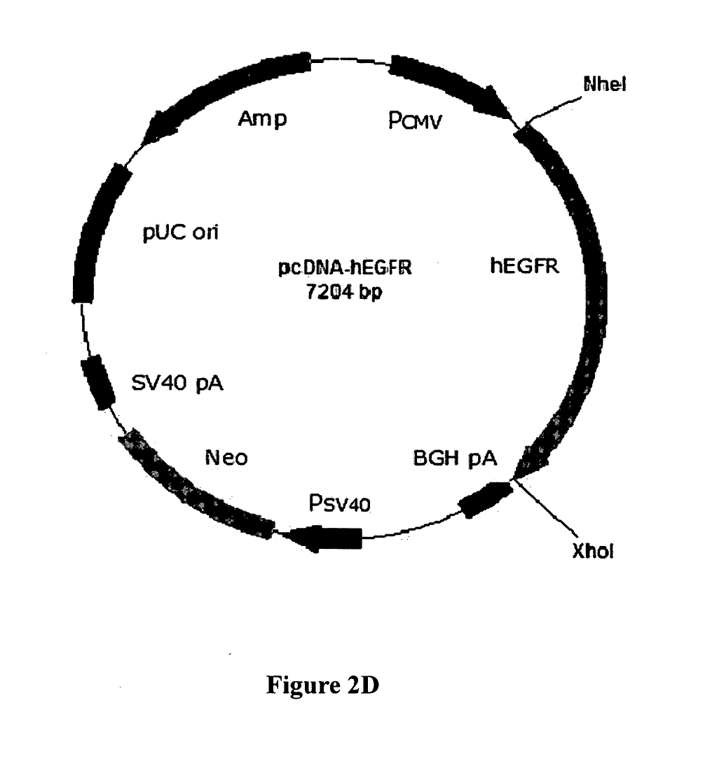

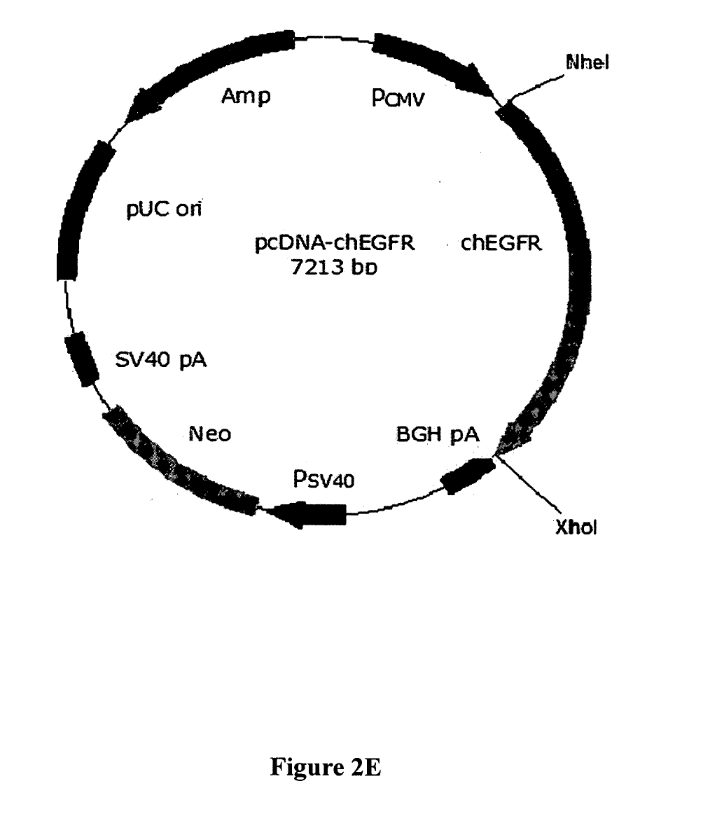

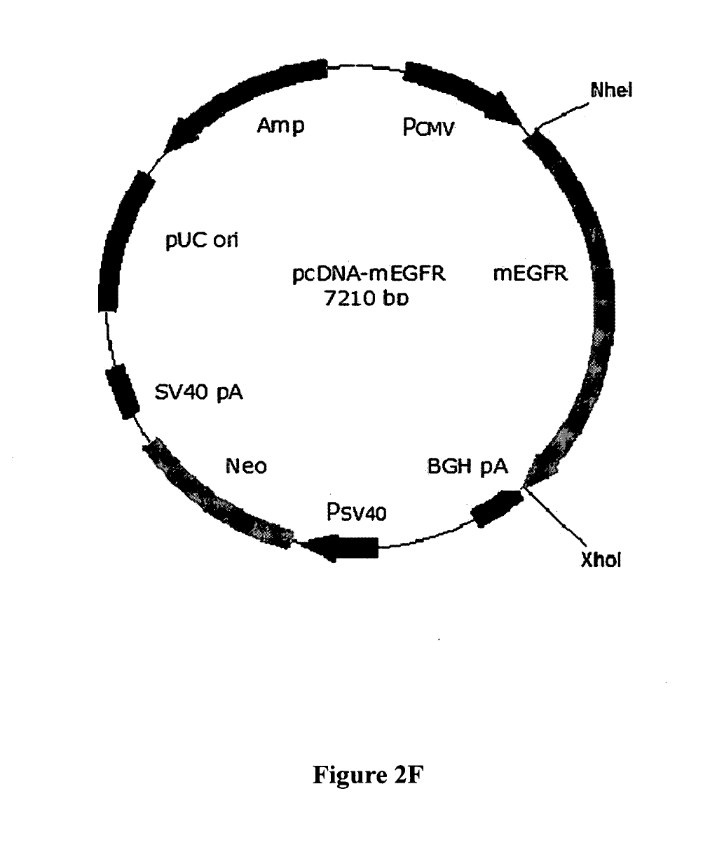

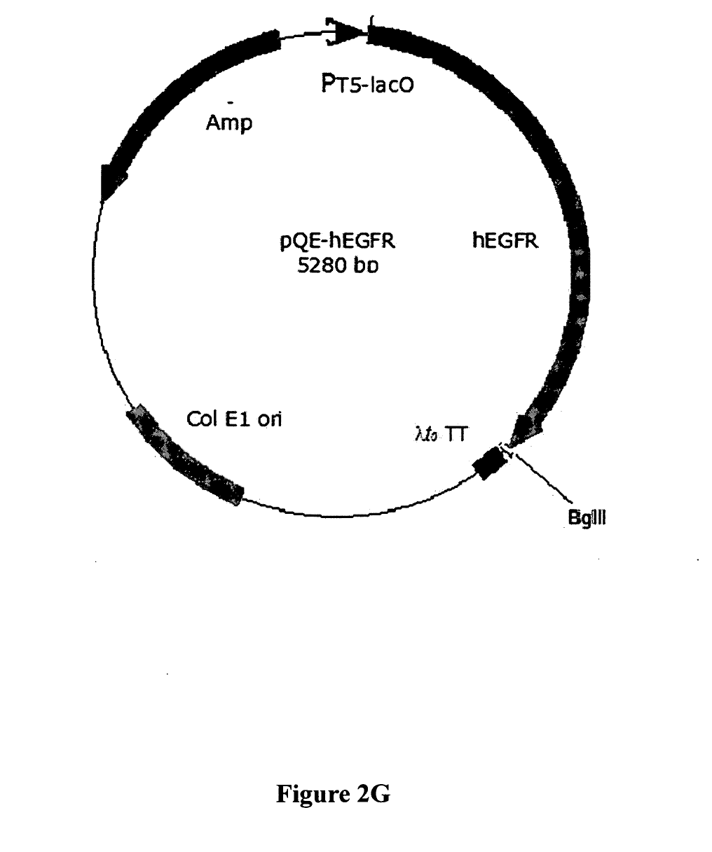

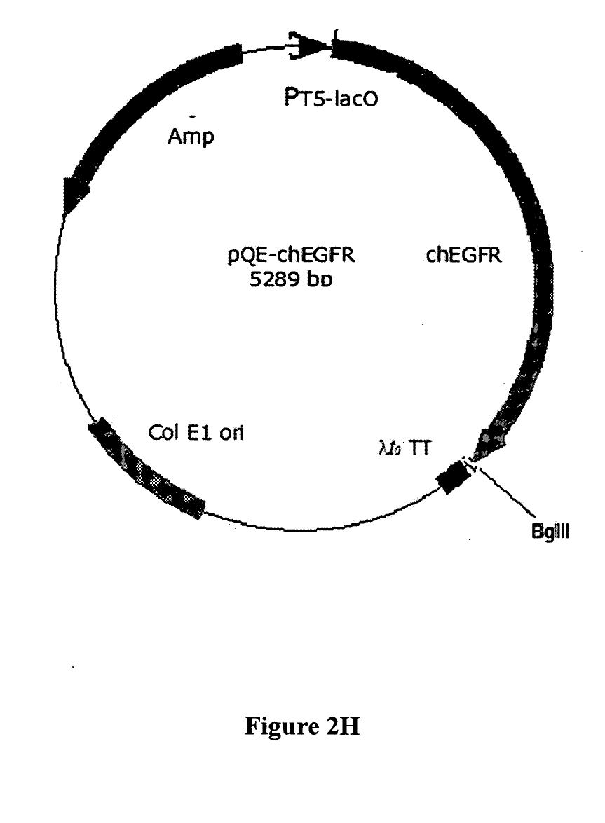

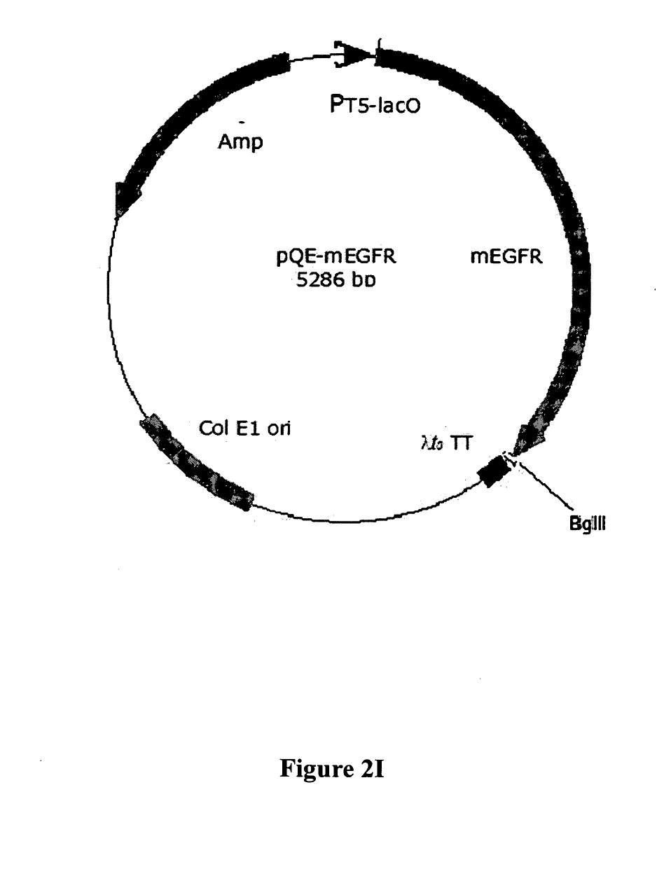

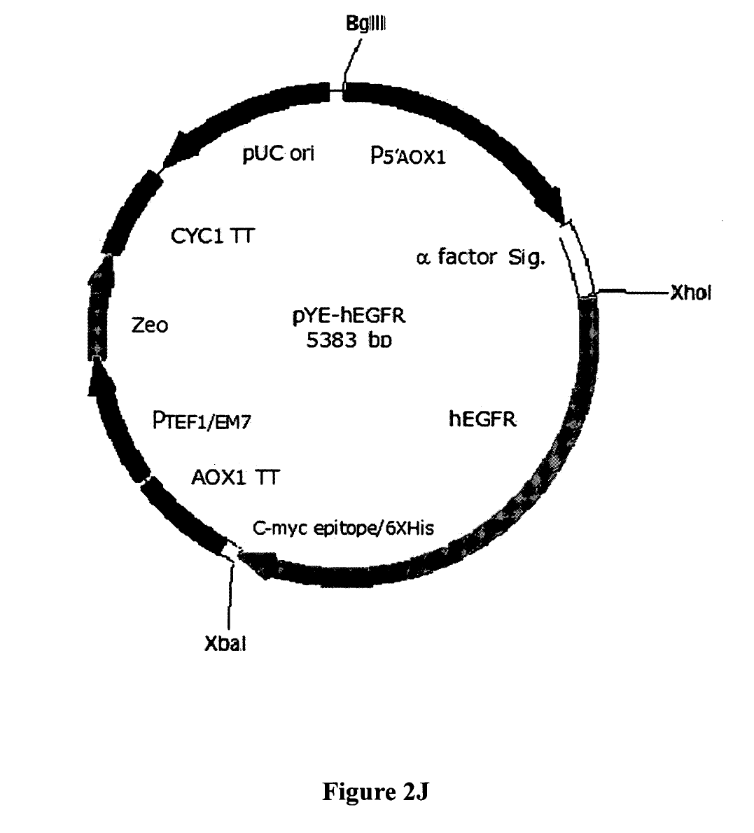

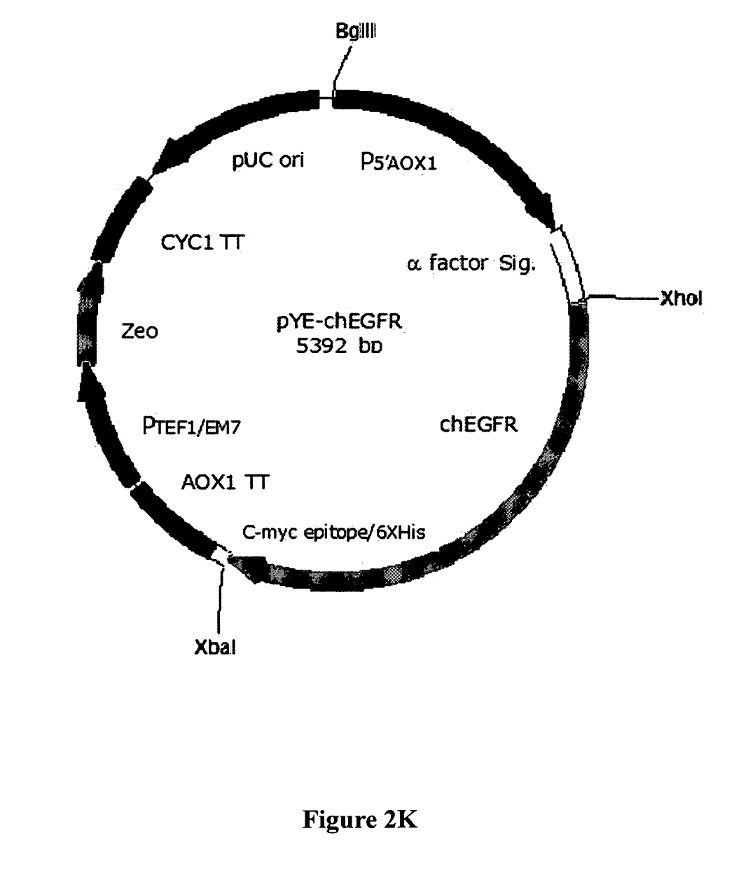

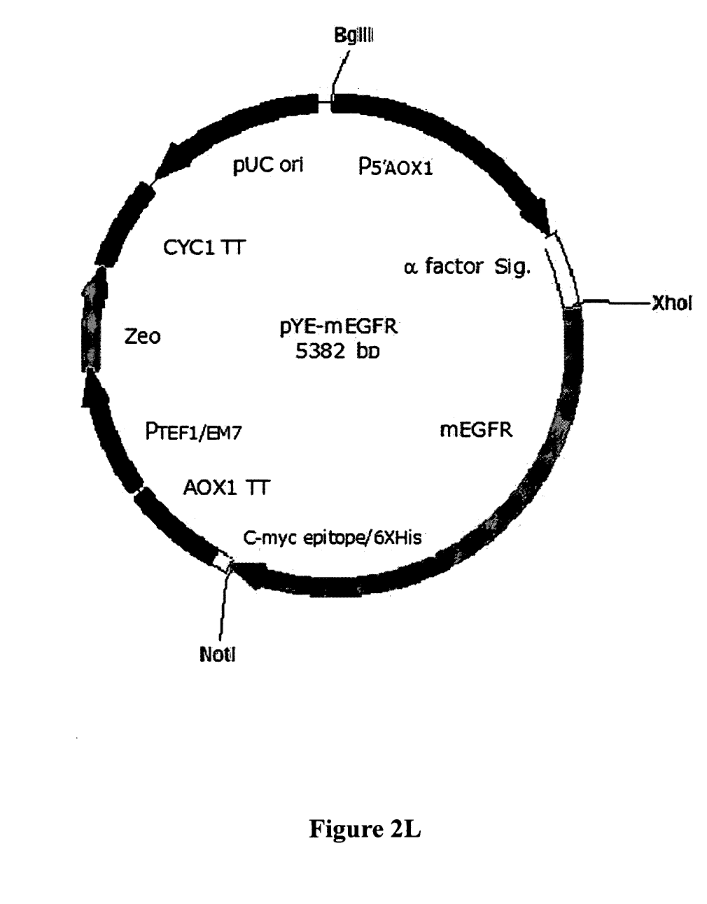

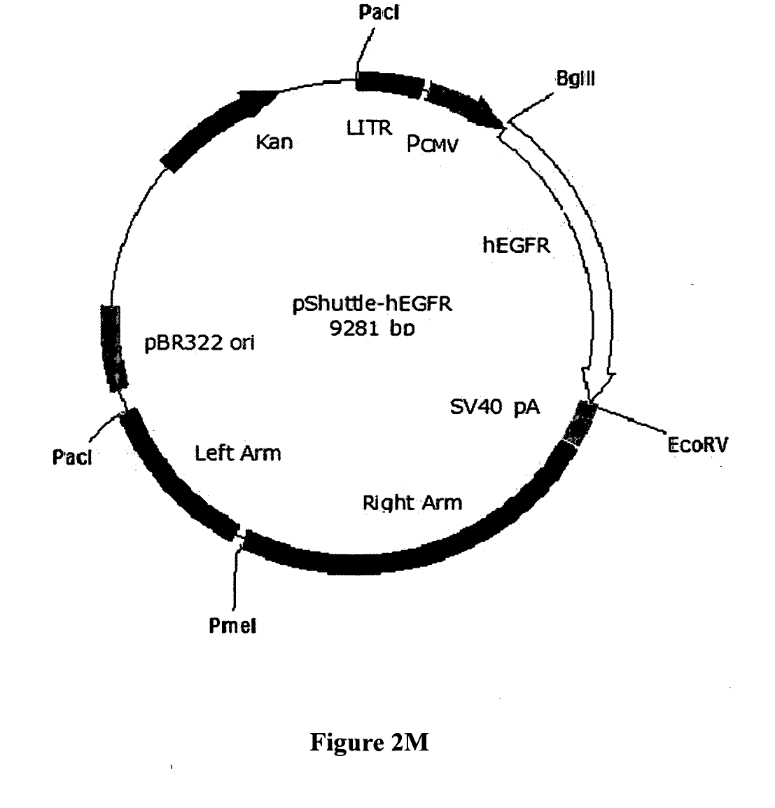

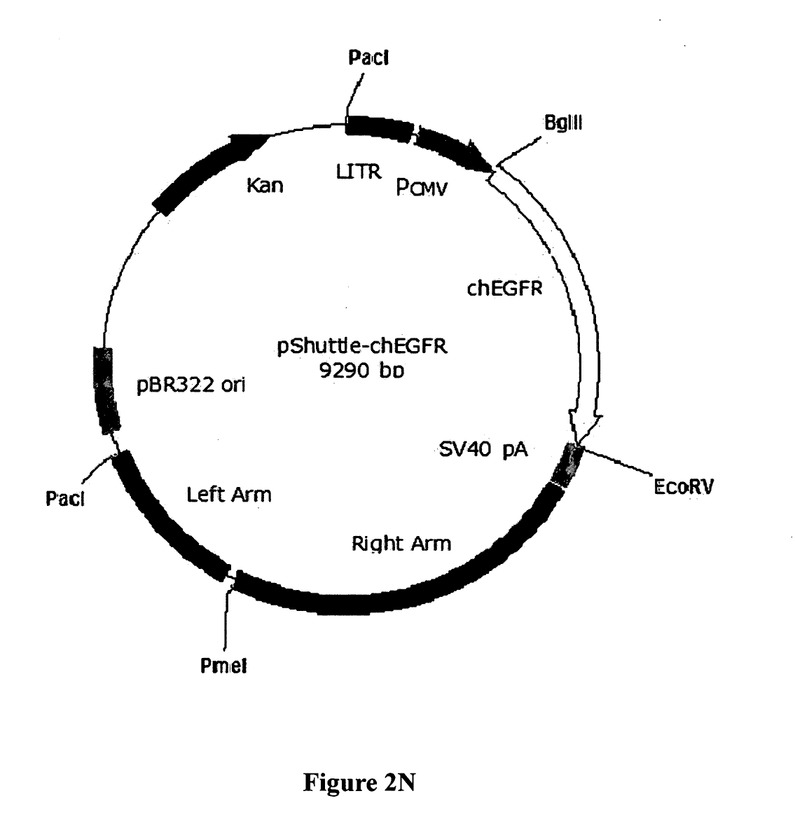

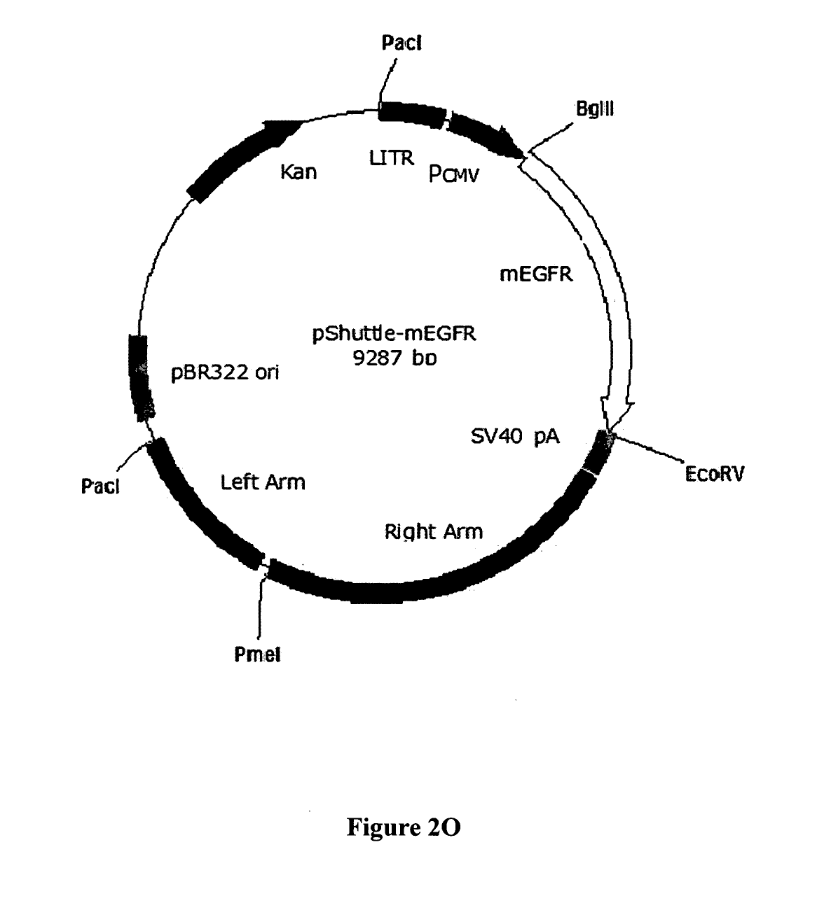

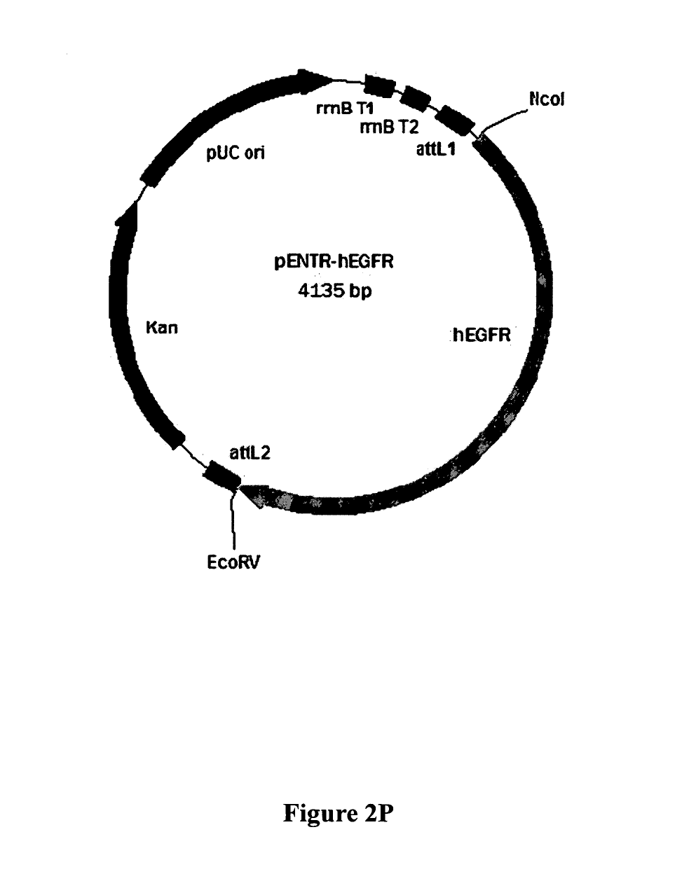

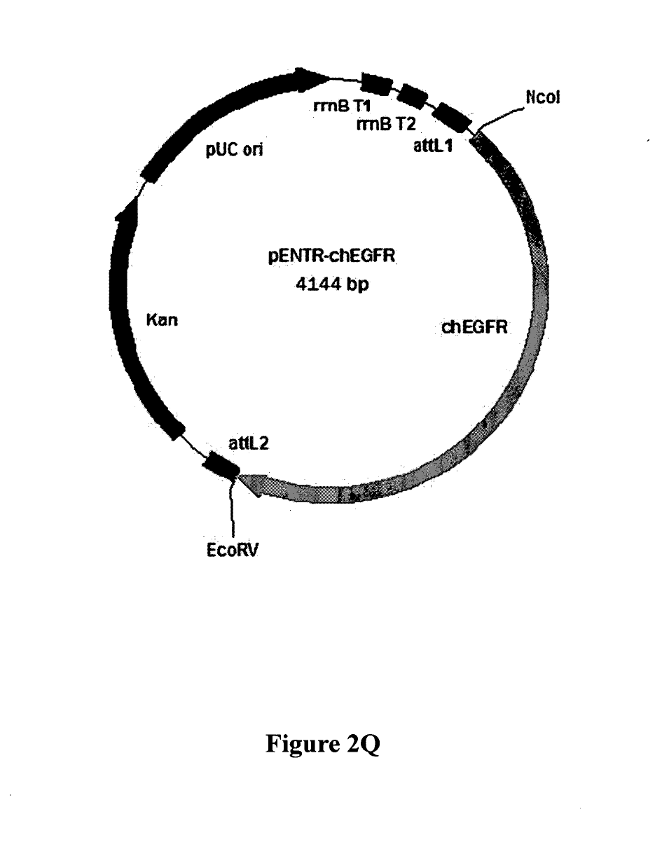

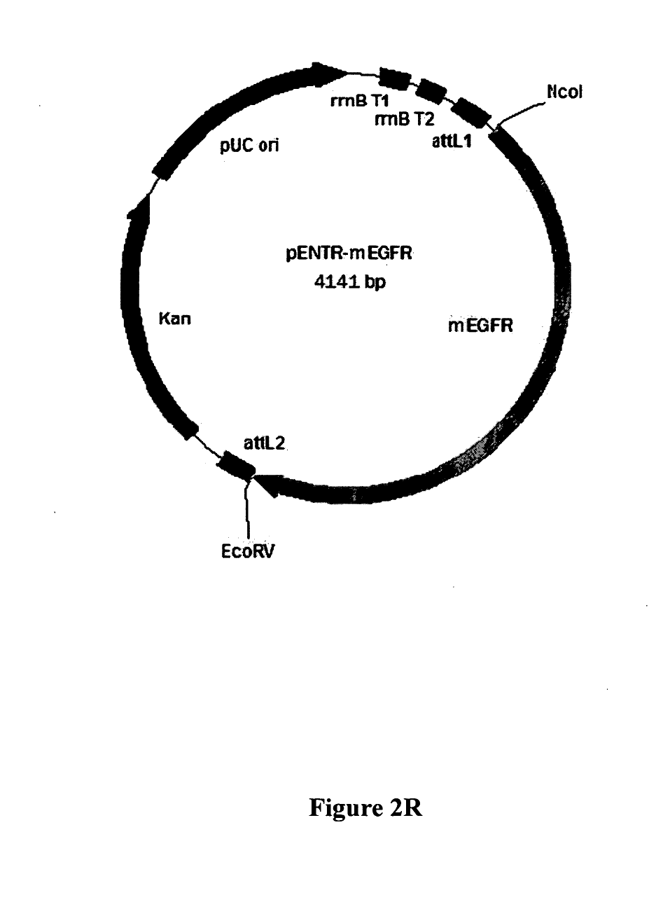

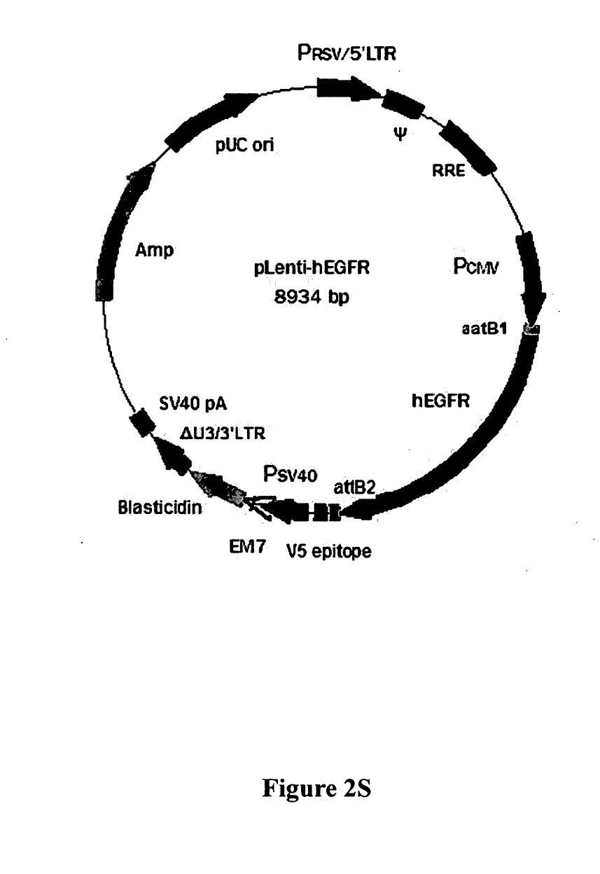

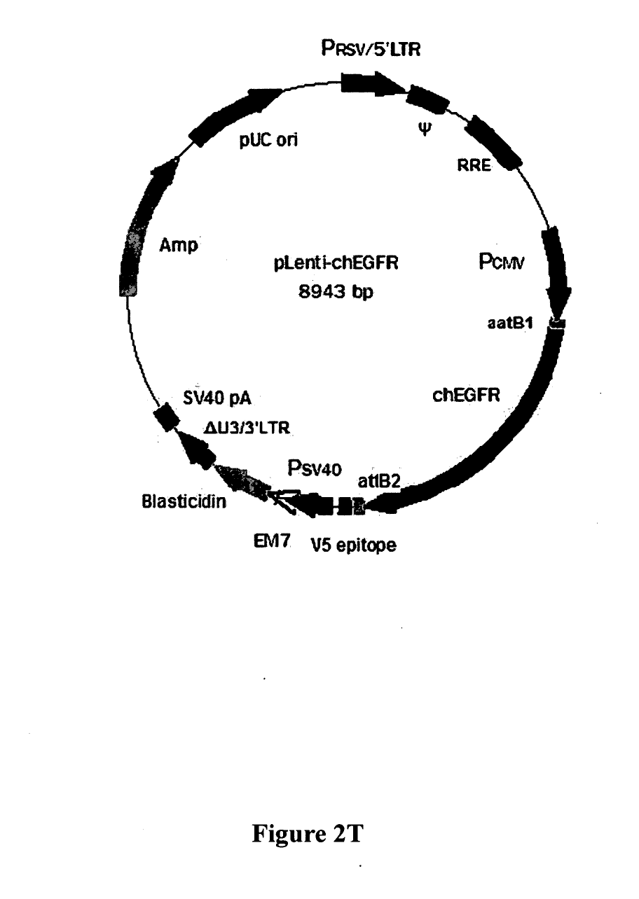

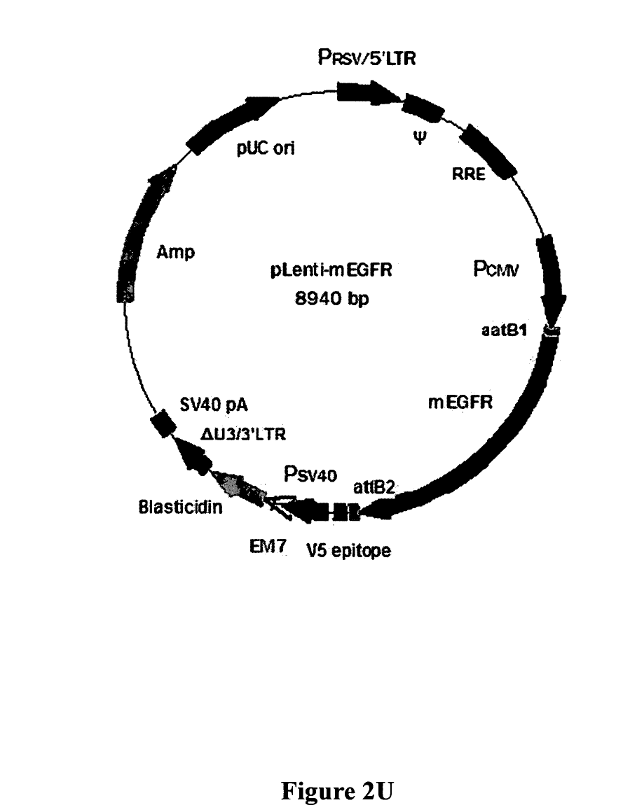

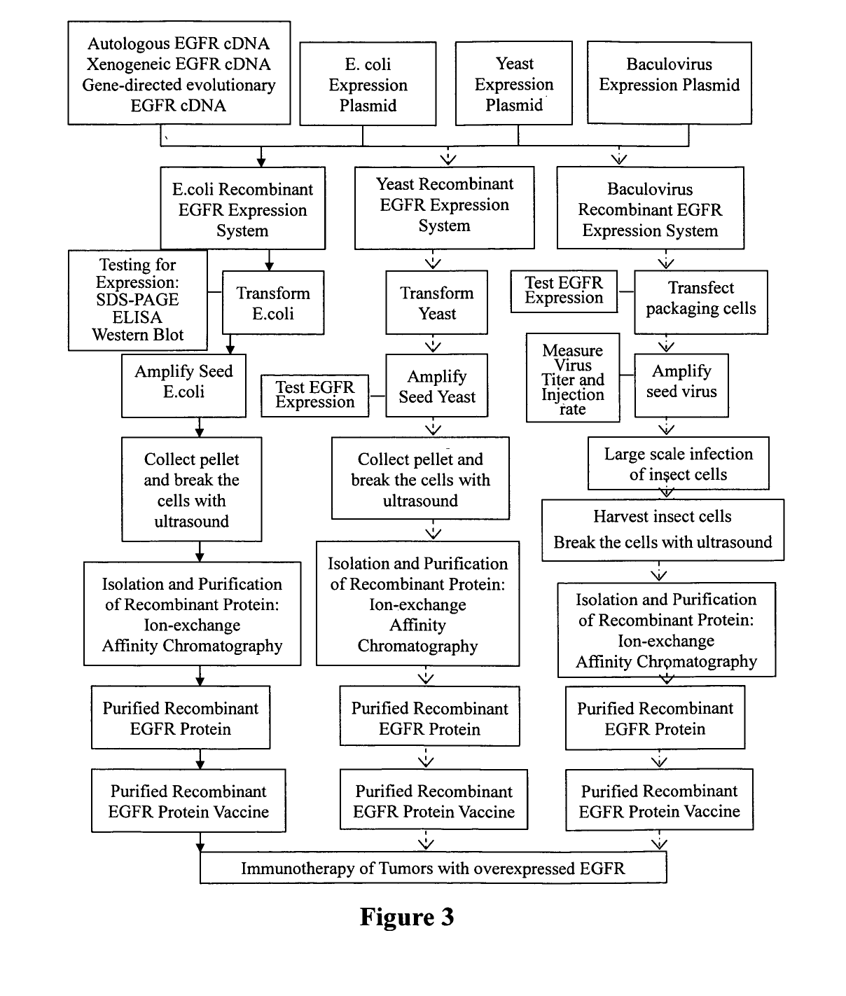

FIG. 2 shows some representative plasmids used to make the EGFR DNA molecular vaccine.

A. pORF based plasmids:

The detailed process for constructing these plasmids is described briefly as follows: we use public database such as GenBank to obtain the cDNA sequences for human, mouse, Chicken, and they are listed herein as SEQ ID NO 1-5,7-9,19, respectively. We designed the following primer based upon the cDNA sequences:

Human Primer:
5′GACCATGGAGGAAAAGAAAGTTTGC3′,
5′ ACGAATTCTTAGGACGGGATCTTAGGCCCA 3′;
Mouse Primer:
5′GACCATGGAGGAAAAGAAAGTCTGC3′,
5′ ACGAATTCTTAATAGATGGTATCTTTGGC 3′;
Chicken Primer:
5′GACCATGGAGGAGAAGAAAGTTTGTC3′,
5′ ACGAATTCTTAAGATGGAGTTTTGGAGCC 3′.

We use the total RNA from human lung cancer cell line A431, mouse lung cancer cell line LL2, and Chick Embryo to undertake RT-PCR amplification, then collect and purify the amplified EGFR fragment (average 1.9 kb in length) using electrophoresis, and then subclone the PCR products. After confirming the sequences of the PCR subclones through sequencing, we digest them using NcoI and EcoRI, collect the 1.9 kb fragments and purify them, and then insert the fragments into the pORF-MCS vector (from the InvivoGen Corporation) which had been digested with NcoI and EcoRI. We select recombiant plasmids. Candidate recombinant plasmids are confirmed by both restriction enzyme digestion analysis and PCR amplification. They are named pORF-hEGFR, pORF-mEGFR and pORF-chEGFR. B. pcDNA Based Plasmids:

For the construction of pcDNA-hEGFR, pcDNA-mEGFR [symbol] pcDNA-chEGFR, the process is similar to the above described process. In this case, we used pcDNA3.1(+) vector made by InvitroGen, and vary the sequences of the PCR primers somewhat from those primers described in the above paragraph.

Human Primer:
5′GAGCTAGCATGGAGGAAAAGAAAGTTTGC3′,
5′ CACTCGAGTTAGGACGGGATCTTAGGCCCA 3′;
Mouse Primer:
5′GAGCTAGCATGGAGGAAAAGAAAGTCTGC3′,
5′ CACTCGAGTTAATAGATGGTATCTTTGGC 3′;
Chicken Primer:
5′GAGCTAGCATGGAGGAGAAGAAAGTTTGTC3′,
5′ CACTCGAGTTAAGATGGAGTTTTGGAGCC 3′;

We used the DNA vaccine constructed and made with human EGFR gene's extracellular domain, and immunized Lewis Lung Cancer Model Mice. We discovered that 8 weeks after immunization, the survival rate of mice injected with the EGFR DNA vaccine is 78%, significantly higher that those mice that were injected with mouse EGFR DNA vaccine (with a survival rate of 25%), and much higher than those mice that were used as a control group (with a survival rate of 10-15%). At the same time, we did not find any pathological changes to the lungs, liver, heart, and kidney of the experimental model mice. Further research was conducted and demonstrated that the induced self-autoimmune response in mice is mainly dependent upon CD4+ T lymphocytes. Testing using the CTL activity test found no target cells-specific cytocidal effect. Immunohistochemistry results showed deposition of autoantibodies in tumor tissues, but no such deposition in non tumor tissues such as lung, liver, etc. The autoantibody in this case is mainly IgG.

What is disclosed in the present invention is different from anti-sense RNA and RNAi molecules designed with EGFR molecule as template. Those molecules can be viewed as a specific example of the EGFR recombinant gene vaccine. Their mechanism of action is not through increasing the immunogenicity of EGFR molecule, thereby inducing anti-EGFR antibody and specific CTL reaction to achieve the goal of blocking the EGFR signal pathway, thus further inducing tumor cell apoptosis, and inhibiting tumor cells growth and metastasis. Rather, these antisense RNA and RNAi molecules act at the DNA and RNA level by directly repressing and prohibiting the expression of EGFR molecules.

3. EGFR Recombinant Protein Vaccine

Protein vaccine is a relatively traditional vaccine, however, protein possess very good immunogenicity. One of the vaccines encompassed in the present invention is recombinant protein vaccine, including those constructed with various expression systems such as E. coli recombinant expression vectors, yeast recombinant expression vectors, baculovirus recombinant expression vectors.

We first prepare the recombinant EGFR constructs as described in Section 2. Namely, using molecular cloning technique such as PCR, RT-PCR, molecular hybridization, we isolated, from various commercially available gene libraries, cDNA library, cell lines, tissue cultures, we cloned and isolated the intracellular domains of the EGFR molecules from a variety of different species. In addition, we can further use the gene directed evolution technique [describe this technique, using a reference is ok] to select EGFR molecules with strong immunogenicity. After confirming the sequences of the extracellular domain of the EGFR cDNAs using sequencing, we constructed prokaryotic plasmid expression systems containing these intracellular domains of the EGFR using molecular cloning technique. We transformed suitable E. coli host, observed and examined their EGFR expression levels. The recombinant EGFR constructs can be analyzed and confirmed using restriction enzyme digestion, SDS-PAGE, and Western Blot. After confirmation, the recombinant EGFR molecules are used to transform E. coli, the transformed E. coli cells are grown in a large quantity in order to produce recombinant proteins. Next, we used low temperature ultra centrifuge to harvest the transformed cells, and then resuspended the cells in PBS solution, and then lyse the cells using ultrasound techniques. We then isolated and purified the recombinant EGFR protein using ion-exchange chromatography and affinity chromatography. The isolated recombinant EGFR protein can be used as protein vaccine to immunize animals.

Representative plasmids for recombinant EGFR protein vaccine expressed in E. coli are shown in FIG. 2B. The detailed process to construction these plasmids is described briefly as follows:

We obtain the cDNA sequences of EGFR from human, mouse, and chicken from public databases such GenBank, corresponding to SEQ ID NO 1-5,7-9,19, respectively. Based upon these sequences, we designed the following primers:

Human Primer:
5′GACCATGGAGGAAAAGAAAGTTTGC3′,
5′ ACAGATCTAGG ACGGGATCTTAGGCCCA 3′;
Mouse Primer:
5′GACCATGGAGGAAAAGAAAGTCTGC3′,
5′ ACAGATCTATAGATGGTATCTTTGGC 3′;
Chicken Primer:
5′GACCATGGAGGAGAAGAAAGTTTGTC3′,
5′ ACAGATCTAGATGGAGTTTTG GAGCC 3′;

We use pORF-hEGFR, pORF-mEGFR, and pORF-chEGFR as template for PCR amplification, then collect and purify the amplified EGFR fragment (average 1.9 kb in length) using electrophoresis, and then subclone the PCR products. After confirming the sequences of the PCR subclones through sequencing, we digest them using NcoI and BglII, collect the 1.9 kb fragments and purify them, and then insert the fragments into the pQE60 vector (from the Qiagen Corporation) which had been digested with two restriction enzymes NcoI and BglII. We select recombiant plasmids. Candidate recombinant plasmids are confirmed by both restriction enzyme digestion analysis and PCR amplification. They are named pQE-hEGFR, pQE-mEGFR, and pQE-chEGFR.

Recombinant EGFR proteins can be produced by methods other than the E. coli recombinant expression systems as described above. For example, one can use the yeast recombinant expression system, baculovirus recombinant expression system. These recombinant EGFR proteins made from all these systems can be used as recombinant protein vaccines. See FIG. 2 of the old application for a flow chart of the construction process of the EGFR recombinant protein vaccine.

Respresentative maps for plasmid expressing EGFR recombinant protein vaccine made from yeast expression system are shown in FIG. 2C. The detailed procedure is described as follows:

Again, as described in the previous sections, we obtain the cDNA sequences of EGFR from human, mouse, and chicken from public databases such GenBank, corresponding to SEQ ID NO 1-5,7-9,19, respectively. Based upon these sequences, we designed the following primers.

Human Primer:
5′ATACTCGAGAAAAGAGAGCTGGAGGAAAAGAAAG3′,
5′ GCTCTAGAATGGCACAGGTGGCACA 3′;
Mouse Primer:
5′ATGCTCGAGAAAAGAGAGTTGGAGGAAAAGAAAGTC3′,
5′ AAGCGGCCGCCATAGATGGTATCT TTG 3′;
Chicken Primer:
5′ATACTCGAGAAAAGAGAGGTGGAGGAGAAGAAAG3′,
5′ CGTCTAGAAGATGGAGTTTTGGAG 3′;

We use pORF-hEGFR, pORF-mEGFR, and pORF-chEGFR as template for PCR amplification, then collect and purify the amplified EGFR fragment (average 1.9 kb in length) using electrophoresis, and then subclone the PCR products. After confirming the sequences of the PCR subclones through sequencing, we digest them using two restriction enzymes XhoI and XbaI (in the case of plasmids containing mouse EGFR, we use XhoI and NotI double enzyme digestion), collect the 1.9 kb fragments and purify them, and then insert the fragments into pPICZ A vector (from Invitrogen Corporation) which had been digested with two restriction enzymes XhoI and XbaI (in the case of plasmids containing mouse EGFR, we use XhoI and NotI double enzyme digestion). We transformed E. coli with these plasmid preparations, and select recombiant plasmids. Candidate recombinant plasmids are confirmed by both restriction enzyme digestion analysis and PCR amplification. They are named yeast expression plasmids: pYE-hEGFR, pYE-mEGFR and pYE-chEGFR.

After digesting these yeast expression plasmids with PmeI to linerize them, we use electric perforation method to transform yeast cell lines X33, or GS115. We use Zeocin resistance to select stable transformants. We use MMH ( Minimal Methanol with histidine , MMH ) and MDH (Minimal Dextrose with histidine, MDH) agar plate) to determine and selecte Mut+ transformants. We select 6-10 Mut+ transformants for small-scale expression, and then use SDS-PAGE, Western Blot, ELISA, etc. to confirm the expressed recombinant protein. We select the Mut+ transformants with the highest expression efficienty, and culture them at a large scale so as to establish yeast expression seed libraries. We use large flasks to culture or ferment the recombinant yeast cell lines, collect the yeast pellets using low temperature centrifuge. After resuspend the pellets n PBS solution, we use ultrasound to break the cells. Then, we used Ion Exchange Chromatography and affinity chromatography to isolate and purify the recombinant EGFR protein. The recombinant EGFR protein derived as such can be used as protein vaccine to immunize subjects. Similarly, yeast recombinant expression plasmids made with EGFR can be produced with other yeast expression systems.

Recombinant protein vaccine has a stronger effect than DNA vaccine in terms of the ability to illicit immune cross-reaction. It can stimulate production of high-titer anti-EGFR antibody and specific CTL activity thereby can inhibit the growth and metastasis of tumor cells.

4. EGFR Recombinant Viral Vaccine

Recombinant viruses are also a good choice as a system for producing molecular vaccine. Molecular vaccines made with these recombinant viral expression systems include recombinant adenovirus vaccine, adenovirus-related viral vaccine, Retroviral viral vaccine, Lenti Virus vaccine, vaccinia virus vaccine, and herpes simplex virus vaccine.

Currently, Adenovirus vector is one of the most effective vectors in tumor gene therapy. It has the advantage of having high titer, safe, and can infect dividing, or non-dividing cells, and it does not integrate into the chromosomes of the host. In addition, adenovirus has relatively strong immunogenicity, which is perhaps a downside in gene therapy, but a strong point in gene immunotherapy. As disclosed in the present invention, recombinant adenovirus-derived EGFR recombinant viral vaccine is one of the most important embodiments.

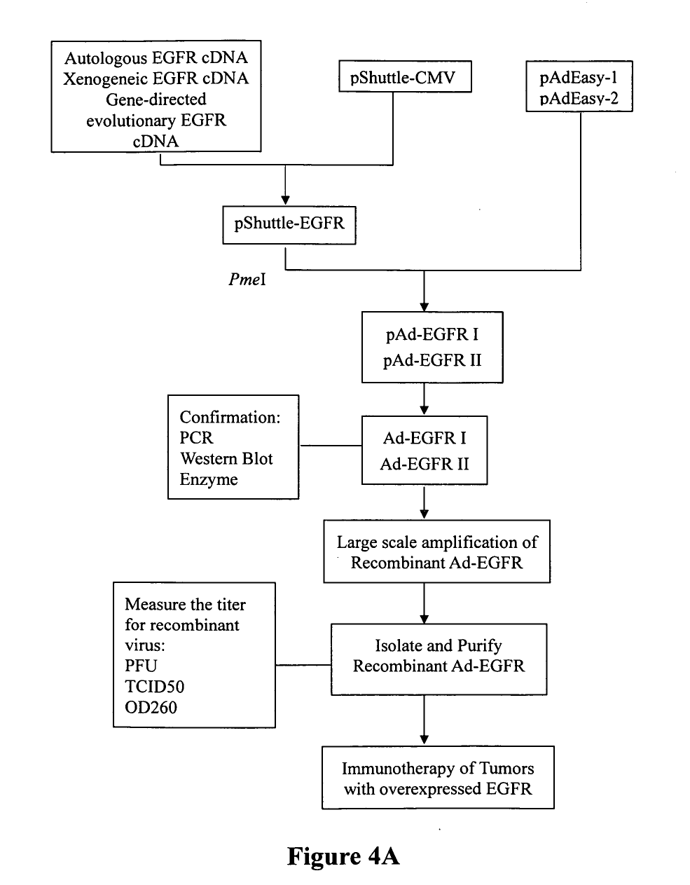

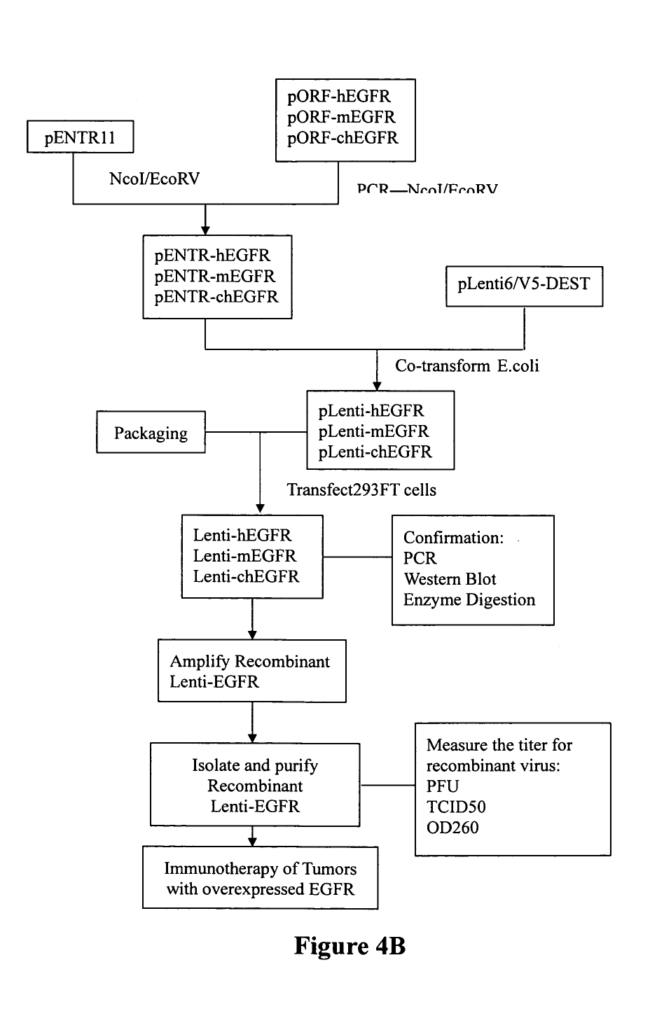

As described in the procedure above, we first cloned various kinds of autologous, xenogeneic, or gene directed evolutionary EGFR cDNA, and then use these cDNAs to construct recombinant adenoviral expression vectors, using molecular biological techniques. We then transfect 293 cells, and harvested the resulting recombinant adenovirus. The recombinant adenovirus is further confirmed using PCR, Western Blot, etc. We took the EGFR recombinant adenoviral vaccine made in large quantity using 293 cells, isolated and purified the recombinant virus using ultracentrifuge, ultra-filtration methods. The EGFR recombinant viral vaccine purified above can be used as vaccine to immunize subjects. See FIG. 4A for a flow chart showing the construction of EGFR recombinant adenoviral vaccine. Because adenovirus can introduce genes effectively, EGFR recombinant viral vaccine made with adenovirus can effectively induce the anti-tumor immune response in the subject, and thereby inhibiting the growth of tumors with over-expressed EGFR.

We construct the extracellular domain of EGFR as described in Example One and Two, and then use the AdEasy Systems to construct recombinant adenovirus in the following manner: we inserted the EGFR segment into the adenovirus vector's precursor expression plasmid pShuttle-CMV in order to construct the precursor expression vector pCMV-EGFR. Then, we co-transform E. coli BJ5183 with two components: one is the PmeI digested adenovirus precursor expression vector pCMV-EGFR, the other is the backbone vector pAdEasy, which contained the adenovirus genome. After the transformation, we obtained recombinant adenovirus vector plasmid pAd-EGFR. We used the restriction enzyme PacI to digest pAd-EGFR, and use Calcium Phosphate-DNA precipitation method to transfect the digested pAd-EGFR into adenovirus packaging cell line 293 cells, resulting the corresponding recombinant adenovirus Ad-EGFR. We used PCR, Western Blot and Restriction enzyme digestion analysis to confirm that the EGFR gene has been indeed incorporated into the adenoviral vector, and that it was expressed effectively in Eukaryotic cells. We used ultracentrifuge to collect large quantity of the recombinant adenovirus Ad-EGFR, and then tested the titer (pfu) of each batch of the recombinant virus using upper layer agarose method, and TCID50 method. We then used 293 cells to produce large amount of confirmed EGFR adenovirus vaccine. We further isolated and purified the recombinant virus using ultracentrifuge and ultrafiltration. The purified EGFR recombinant adenovirus can be used a vaccine to immunize subject animals.

As a representative of the EGFR Recombinant Virus Vaccine, we described below the construction of EGFR adenovirus vaccine, which is based upon the AdEasy system. The process is as follows:

We obtain the cDNA sequences of EGFR from human, mouse, and chicken from public databases such GenBank, corresponding to SEQ ID NO 1-5,7-9,19, respectively. Based upon these sequences, we designed the following primers:

Human Primer:
5′GAAGATCTATGGAGGAAAAGAAAGTTTGC3′,
5′ ACGATATCTTAAGGACGGGATCTTAGGCCCA 3′;
Mouse Primer:
5′GAAGATCTATGGAGGAAAAGAAAGTCTGC3′,
5′ ACGATATCTTAATAGATGGTATCTTTGGC 3′;
Chicken Primer:
5′GAAGATCTATGGAGGAGAAGAAAGTTTGTC3′,
5′ ACGATATCTTAAGATGGAGTTTTGGAGCC 3′;

We use pORF-hEGFR, pORF-mEGFR, and pORF-chEGFR as template for PCR amplification, then collect and purify the amplified EGFR fragment (average 1.9kb in length) using electrophoresis, and then subclone the PCR products. After confirming the sequences of the PCR subclones through sequencing, we digest them using two restriction enzymes BglII and EcoRV double enzyme digestion, collect the 1.9 kb fragments and purify them, and then insert the fragments into pShuttle-CMV vector (made by Quantum Biotechnologies) which was pre-digested with BglII and EcoRV. We select recombiant plasmids. The resulting candidate recombinant plasmids are confirmed by both restriction enzyme digestion analysis and PCR amplification. They are named Adenovirus shuttle expression plasmids: pShuttle-hEGFR, pShuttle-mEGFR, and pShuttle-chEGFR. See FIG. 2D for plasmid maps of these Adenovirus shuttle expression plasmids.

We then take the various Adenovirus shuttle expression plasmids as described before, digest them with PmeI enzyme, and co-transform E. coli BJ5183 cells with backbone vector containing the Adenovirus genome pAdEasy-1 or pAdEast-2. The resulting recombinant Adenovirus vector plasmids are named pAd-hEGFR, pAd-mEGFR and pAd-chEGFR. These Adenovirus vector plasmids are digested with PacI enzyme, and then using the Calcium-phosphate-DNA coprecipitation method, they are transfected into the Adenovirus packaging cell line 293 cells. The resulting recombinant Adenoviruses are called Ad-hEGFR, Ad-mEGFR, and Ad-chEGFR. PCR, Western blot and other methods are used to confirm that the EGFR gene has been indeed integrated into the Adenovirus vector, and that EGFR has been correctly, and efficiently expressed in Eukaryotic cells.

Depending upon the difference of the Adenovirus Genome, the recombinant Adenovirus vaccine can be classified into two groups: the first group is called the generation I of EGFR Recombinant Adenovirus, resulting from the recombination between Adenovirus Shuttle expression plasmid pShuttle-EGFR and AdEasy-1 recombinant, thus named Ad-hEGFR I, Ad-mEGFR I and Ad-chEGFR I. The second group is called the generation II of EGFR Recombinant Adenovirus, resulting from the recombination between Adenovirus Shuttle expression plasmid pShuttle-EGFR and AdEasy-2 recombinant, thus named Ad-hEGFR II, Ad-mEGFR II and Ad-chEGFR II.

In addition, the EGFR Recombinant Virus Vaccine can be modified specifically to increase its targeting ability with the compound Mannan.

Lentivirus vector is a new generation gene thearpy vector. It originated from lentivirus which has a HIV-1 replication deficiency. It is different from the traditional reverse transcriptase virus vector originated from the Moloney Leukemia Virus (MoMLV). The difference is that Lentivirus vector can effectively transfect both dividing and non-dividing mammalian cells, and it has good biological safety. In addition, Lentivirus vector differs from Adenovirus vector in that Lentivirus vector can integrate into the host cell's chromosomes, and thus enabling the introduced exogenous gene's stable and long-term expression. Recombinant Lentivirus vector is also an important variation in EGFR recombinant virus vaccine.

The process to make EGFR Recombinant Lentivirus Vaccine is similar to what is described with respect to Adenovirus. That is, we first clone various autologous, xenogeneic, or gene-directed evolution EGFR cDNA, and then using molecular biology technique, we construct its Recombinant Lentivirus expression vector, then transfect 293FT cells, resulting in recombinant Lentiviruses. The recombinant Lentiviruses are confirmed using PCR, Western Blot, etc. Further, after confirmation, we use 293FT cells to amplify large quantity of the EGFR Recombinant Lentivirus vaccine, and further isolate and purify the recombinant Lentivirus using ultracentrifuge and ultrafiltration. The purified EGFR Recombinant Lentivirus can be used as vaccine to immunized subjects. See FIG. 4B for a flow chart of the construction of Recombinant Lentivirus vaccine.

As an illustration of the process of making EGFR Recombinant Lentivirus, we use the ViraPower Lentiviral Gateway Expression Kit made by Invitrogen Corporation. The detailed process is as follows: we first search public database such as GenBank to obatin cDNA sequences of EGFR molecule of human, mouse and chicken. Their sequences are listed as SEQ ID NO 1-5, 7-9, 19, respectively.

We then designed the following PCR primers:

Human Primer:
5′GACCATGGAGGAAAAGAAAGTTTGC3′,
5′ ACGATATCAGGACGGGATCTTAGGCCCA 3′;
Mouse Primer:
5′GACCATGGAGGAAAAGAAAGTCTGC3′,
5′ ACGATATCATAGATGGTATCTTTGGC 3′;
Chicken Primer:
5′GACCATGGAGGAGAAGAAAGTTTGTC3′,
5′ ACGATATCAGATGGAGTTTTGGAGCC 3′;

We use pORF-hEGFR, pORF-mEGFR, and pORF-chEGFR as template for PCR amplification, then collect and purify the amplified EGFR fragment (average 1.9 kb in length) using electrophoresis, and then subclone the PCR products. After confirming the sequences of the PCR subclones through sequencing, we digest them using two restriction enzymes NcoI and EcoRV double enzyme digestion, collect the 1.9 kb fragments and purify them, and then insert the fragments into pENTR11 vector ( made by Invitrogen) which was pre-digested with NcoI and EcoRV. We select recombiant plasmids. The resulting candidate recombinant plasmids are confirmed by both restriction enzyme digestion analysis and PCR amplification. They are named as pENTR-hEGFR, pENTR-mEGFR, and pENTR-chEGFR. See FIG. 2E for plasmid maps of these Lentivirus expression plasmids.

We then take the various Lentivirus expression plasmids as described before, co-transform E. coli DH5 cells together with backbone vector containing the Lentivirus genome pLenti6/V5-DEST. The resulting recombinant Lentivirus vector plasmids are named pLenti-hEGFR, pLenti-mEGFR and pLenti-chEGFR. See also FIG. 2E. We then mix these Recombinant Lentivirus vector plasmids with packaging mix, the ViraPower Packaging Mix, and then using the Calcium-phosphate-DNA coprecipitation method, the Recombinant Lentivirus vector plasmids are transfected into the Lentivirus packaging cell line 293FT cells. The resulting recombinant Lentiviruses are called Lenti-hEGFR, Lenti-mEGFR and Lenti-chEGFR. PCR, Western blot and other methods are used to confirm that the EGFR gene has been indeed integrated into the Lentivirus vector, and that EGFR has been correctly, and efficiently expressed in Eukaryotic cells.

5. Modification and Improvement of EGFR Molecular Vaccine

What is described above is a basic procedure of preparing recombinant tumor associated protein vaccine, and in the preferred embodiment, recombinant EGFR molecular vaccine. We now describe more embodiments below which include modifications and improvements that can enhance the efficacy and specificity of the EGFR molecular vaccine.

1) Choice of Adjuvant for the EGFR Molecular Vaccine

Suitable adjuvant is carriers for vaccines that can increase the effective immune response of the vaccine. Different vaccines have different adjuvant. In the present invention, for the EGFR DNA vaccine, we use Freund's adjuvant and liposome. For the EGFR recombinant protein vaccine, we mainly use aluminum adjuvant, and for the EGFR recombinant viral vaccine, generally, no adjuvant is used.

2) EGFR Gene-Modified Vaccine

Gene-modified vaccine refers to cell vaccine derived from human, other xenogeneic organism's cell lines transfected with EGFR molecule. These cell vaccines include vaccine made using various stably transformed tumor cell lines, vascular endothelial cells and dendritic cells. They also include vaccines made by using tumor cells or tissues infected by recombinant viruses. EGFR gene-modified vaccine is a particular example of EGFR molecular vaccine.

As discussed above in the present invention, EGFR is highly expressed in a variety of tumors such as lung cancer, breast cancer, ovarian cancer, colorectal cancer, prostate cancer, stomach cancer, bladder cancer, head and neck squamocarcinoma, and glioma, etc. However, EGFR is tolerated in all these tumors and thus cannot elicit immune response. However, by using gene-modified vaccine, we can break the immune tolerance in these cells, and thus induce anti-EGFR immune response. The procedure of using gene-modified vaccine is similar as discussed in the above section. We use Eukaryotic plasmids containing recombinant EGFR from a variety of different sources to transfect various kinds of tumor cell lines such as lung cancer line A431, breast cancer cell line MCF7, etc, or, vascular endothelial cells. After transfection, we select stable transfectants containing stably expressed EGFR molecules. We harvest the transfectants, and generate cellular vaccines using paraformaldehyde fixation method. In addition, we can also use various EGFR recombinant viral vaccine from different sources to infect tumor cell lines, tumor tissue, and tumor vascular endothelial cells, etc. We then can generate cellular vaccine using the same paraformaldehyde fixation method.

3). Stable EGFR Transformants of Commensal Bacteria

EGFR molecular vaccine also includes live vaccine, which is the stable EGFR transformants of commensal bacteria.

To produce these live vaccines, we use prokaryotic recombinant expression plasmids constructed with EGFR from various different sources such as autologous, xenogeneic, and gene-directed evolutionary EGFR. We use these plasmids to transform intestinal commensal bacteria such as Bifidobacterium. We then select stable transformant. These stable EGFR transformants of commensal bacteria can continuously secrete exogenous EGFR molecules, thereby inducing the body's immune response, thus they are categorized as live vaccine. It is a particular example of the EGFR molecular vaccine.

4) Targeted Nano Particles for EGFR Molecular Vaccine

As shown in the above sections, various embodiments of tumor associated protein molecular vaccine, such as EGFR molecular vaccine can be applied to induce effective immune response to inhibit tumor growth. However, these pure recombinant molecular vaccines, such as EGFR molecular vaccine (including EGFR recombinant gene vaccine, protein vaccine and viral vaccine) still can be improved to enhance its specificity and immunogenicity. The present invention discloses the use of nanotechnology to enhance and modify the EGFR molecular vaccine, by using targeted nanoparticles in making the EGFR molecular vaccine. The protocol includes the following steps:

We use EGFR molecules, either its DNA or protein form, as target antigen molecule, we use biological molecules such as liposome, degradable polymer biological materials such as poly DL-lactide-co-glycolide polymer (PLGA) as nano materials, we use MIP-3α, and Flt3-L as modifying genes, we use dendritic cells as target cells, with these materials, we can construct a new type of targeted EGFR vaccine nanoparticles. There are two groups of these nano particles: The first group includes nanoparticles made with nano liposomes, or, PLGA, as materials to manufacture nanoparticles carrying plasmids which can express EGFR in a high efficiency manner. The outside surface of these nano particles are modified with Flt3-L, or Mannan. The second group includes nanoparticles made with liposome, or PLGA, as basic materials, such nanoparticles can carry plasmids with highly expressed MIP-3α genes, and at the same time, the nano particles also contain other plasmids highly expressing EGFR molecules.

EGFR molecular vaccine modified with targeted nano particles can further improve effectively the immunogenicity of EGFR, thereby can induce a stronger anti-tumor immune response, as compared to regular EGFR molecular vaccine without the modification of targeted nano particles. See FIG. 4 of the priority application for schematic description of targeted nano particles containing EGFR molecular vaccine.

The targeted nanoparticles in the present invention come in a variety of forms, including
1) mannan-modified nanoparticle, including mannan-modified recombinant adenoviral EGFR vaccine and protein vaccine, mannan-modified liposome EGFR gene vaccine and protein vaccine)
2) gene-targeted nanoparticle, including gene-targeted nanoliposome EGFR vaccine, gene-targeted nano-PLGA EGFR vaccine, and gene-targeted adenoviral EGFR vaccine, which express EGFR and MIP-3β simultaneously,
3) recombinant adenoviral EGFR vaccine targeting cancer vescular endothelial cell, including RGD-modified recombinant adenoviral EGFR vaccine)

As disclosed in the present invention, the diameter of the nanoparticles is generally less than 500 nm, and they can be classified into three sizes: 200-500 nm, 100-200 nm, and 50-100 nm. It is preferable to use nanoparticles with diameter ranging from 50 nm to 100 nm, and the nano peak value at around 80 nm.

The following procedure applies to the preparation of Mannan-modified Recombinant Adenovirus EGFR vaccine: we first use standard protocol to obtain and amplify EGFR Recombinant Adenovirus (either Generation I or II), and then use chromatography or ultracentrifuge to purify the recombinant adenovirus. Next, we dissolve 70 mg Mannan from Sigma into 5 ml 0.1M phosphate buffer (pH6.0) to reach a final concentration of 14 mg/ml, and then add 45 ml 0.01M Sodium Periodate solution, and mix and oxidize at 4° C. for 60 minutes. After that, we add 10 μl glycol, and incubate for 30 minutes at 4° C., resulting in Oxidative Mannan (Ox-M) mixture.

We then load the Ox-M mixture onto Sephadex-G25 columns previously balanced with bicarbonate buffer (pH6.0-9.0) and perform chromatography, with Ox-M being eluted into 2 ml sized empty vessel. After that, we mix the purified Ox-M with 1×10¹⁴ Recombinant Adenovirus particles at room temperature overnight, obtaining the needed Ox-M-Adenovirus. We then add 1 mg/ml Sodium Borohydride to the Ox-M-Adenovirus, leave at room temperature for 3 hours, forming Reductive Mannan Adenovirus (Red-M-Adenovirus). Both Ox-M-Adenovirus and Red-M-Adenovirus are desalted by ultrafiltration, and condensed, filtering out bacteria. They are stored in small test tubes, and preserved at −80° C. The Mannan-modified Recombinant EGFR Adenovirus can be used as vaccine to immunize a subject.

As the size of the Adenovirus is around 80 nm, it is considered a natural nanoparticle. Adenovirus particles can be modified so that the Adenoviral fiber protein can express the tri-peptide: RGD. The RGD tripeptide has specific ability to target tumor vascular endothelial cells. Recombinant Adenovirus EGFR vaccine modified with RGD can be viewed as a natural targeted EGFR vaccine nanoparticle.

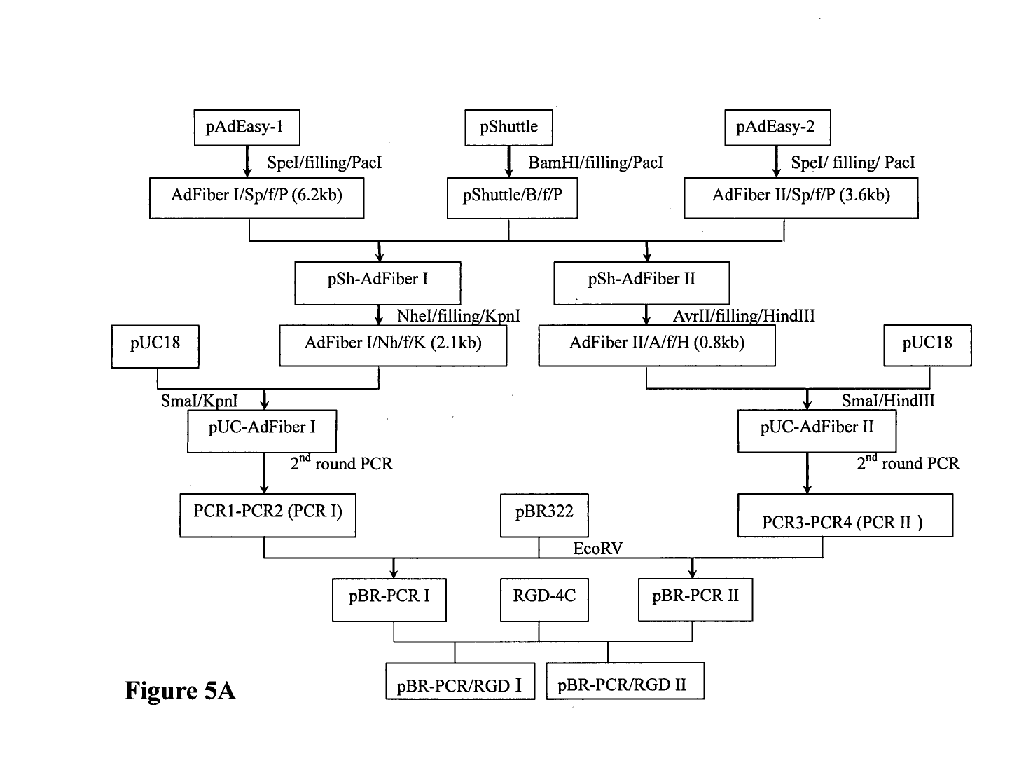

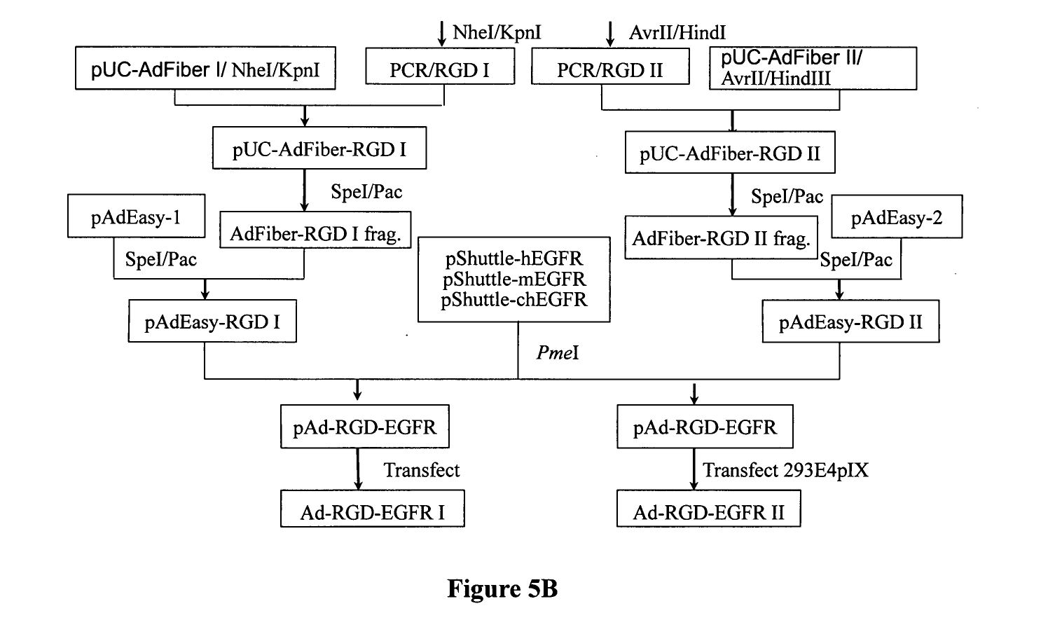

In the present invention, we utilize the AdEasy system to construct the RDG modified Adenovirus Recombinant EGFR vaccine. The detailed process is shown step by step in FIG. 5.

The detailed process is as follows: we digest the Adenovirus backbone plasmid pAdEasy-I and pAdEasy-2 with restriction enzyme SpeI (Sp), and then use T4 DNA Polymerase to fill in the ends (filling , f) so as to make them blunt, and then digest the filled-in product with PacI (P), and recover the 6211 bp and 3579 bp fragments using electrophoresis, and name them AdFiber I/Sp/f/P and AdFiber II/Sp/f/P, respectively. These fragments contain the intact Adenovirus fiber protein gene. Separately, prepare the pSuttle vector by digesting it with BamHI first, and then fill in with T4 DNA Polymerase, and then digest with PacI. After such BamHI/filling/PacI-digestion treatment, the vector is ready to be inserted with the AdFiber I/Sp/f/P and AdFiber II/Sp/f/P fragments. The resulting plasmids are named pSh-AdFiber I and pSh-AdFiber II, respectively.

We then digest pSh-AdFiber I with NheI enzyme, fill in with T4 DNA polymerase, and digest again with KpnI enzyme (NheI/filling/KpnI), recover, using electrophoresis, the 2090 bp fragment called AdFiber I/Nh/f/K; insert this fragment into a pUC18 vector which had been pre-digested with SmaI and KpnI double enzyme digestion, resulting in the recombinant plasmid named pUC-AdFiber I.

On the other hand, pSh-AdFiber II is digested with AvrII enzyme, then filled in with T4 DNA polyerase, and then digested with HindIII (AvrII/filling/HindIII) , using electrophoresis, recover the 838 bp fragment, called AdFiber I/A/f/H. Insert this fragment into a pUC vector which had been previously digested with SmaI and HindIII double enzyme digestion, resulting in new plasmids named pUC-AdFiber II.

Next, we designed a series of PCR primers so as to use pUC-AdFiber I and pUC-AdFiber II as templates to amplify the Adenovirus knob, (Ad-knob) gene sequences. The primers used are, respectively:

| | |
|---|---|
| F1 | (5′ GAAAGCTAGC CCTGCAAACATCA 3′), |
| R1 | (5′ ACTCCCGGGAGTTGTGTCTCCTGTTTCCTG 3′), |
| F2 | (5′ ACTCCCGGGAGTGC ATACTCTATGTCA 3′), |
| R2 | (5′ TATGGTAC CGGGAGGTGGTGA 3′), |
| F3 | (5′ AACCTAGGGAGGTTAACCTAAGCACTG 3′), |
| and | |
| R3 | (5′ CTCAAGCTTTTTGG AATTGTTTGA 3′). |

Using primer F1-R1, F2-R2, F3-R1 and F2-R3, respectively, for the first round PCT , we obtain products PCR1, PCR2, PCR3 and PCR4. Again, using F1-R2 and F3-R3 as primers, and using the amplified products from the first round, PCR1 and PCR2, PCR3 and PCR4 as templates, we undertake the second round of PCR amplification, resulting in PCR products PCR1-PCR2 (PCR I), PCR3-PCR4 (PCR II) . We take the PCR I and PCR II from the amplification in the second round, insert them into pBR322 vector that had been previously cut with EcoRV. The resulting recombinant plasmids are named pBR-PCR I and pBR-PCR II.

The sequence is the RGD-4C duplex is as follows:

5′ TGTGACTGCCGCGGAGACTGTTTCTGC 3′
3′ ACACTGACGGCGCCTCTGACAAAGACG 5′

We insert the RGD-4C into the pBR-PCR I and pBR-PCR II vectors, where the vector was previously digested with SmaI. The resulting recombinant plasmids are named pBR-PCR/RGD I and pBR-PCR/RGD II. The sequences of the recombinant plasmids are confirmed using sequencing. Cut pBR-PCR/RGD I with NheI/KpnI double digestion , using electrophoresis, rcover the PCR/RGD I fragment. Insert the fragment into the pUC-AdFiber I vector which had been double digested using NheI/KpnI. The resulting recombinant plasmids are called pUC-AdFiber-RGD I.

Similarly, we use AvrII/HindIII to double digest pBR-PCR/RGD II, and using electrophoresis, recover the PCR/RGD II fragment. Again, insert the fragment into pUC-AdFiber II vectors previously digested with double enzume AvrII/HindIII, the resulting plasmid is named pUC-AdFiber-RGD II.

Afterwards, we use SpeI/PacI double enzyme to digest pUC-AdFiber-RGD I and pUC-AdFiber-RGD II vector, and using electrophoresis, recover the AdFiber-RGD I, and AdFiber-RGD II fragments. Insert the fragment into pAdEasy-1, pAdEasy-1 both of which were previously digested with SpeI/PacI, the resulting recombinant plasmids are called pAdEasy-RGD I, and pAdEasy-RGD II, respectively.

Next, we first take pShuttle-hEGFR, pShuttle-mEGFR and pShuttle-chEGFR as described above, and linerize them with PmeI. After that, we co-transform the E. coli BJ5183 cells with these Shuttle-EGFR plasmids together with pAdEasy-RGD I, and pAdEasy-RGD II, respectively. The resulting recombinant plasmids are named Adenovirus plasmids pAd-RGD-EGFR I, and pAd-RGD-EGFR II.

We used the Adenovirus plasmid pAd-RGD-EGFR I to transfect 394 cells, resulting in recombinant Adenovirus named Ad-RGD-EGFR I. Similarly, we transfect Adenovirus plasmid pAd-RGD-EGFR II to 293E4pIX cells, resulting in recombinant Adenovirus named Ad-RGD-EGFR II. After purification, Ad-RGD-EGFR I and Ad-RGD-EGFR II can be used as vaccine to immunize subjects, and more importantly, these vaccine possess specificity targeting tumor vascular endothelial cells.

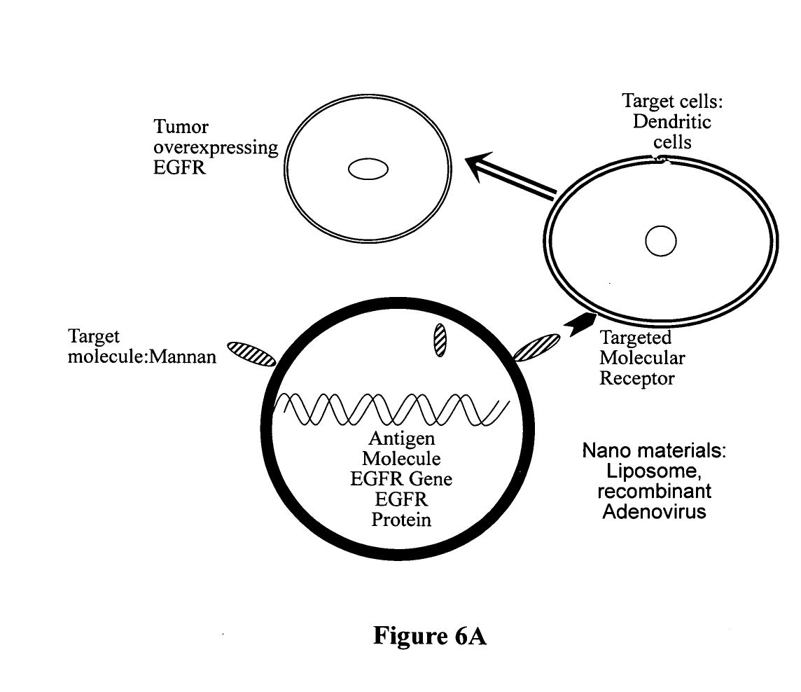

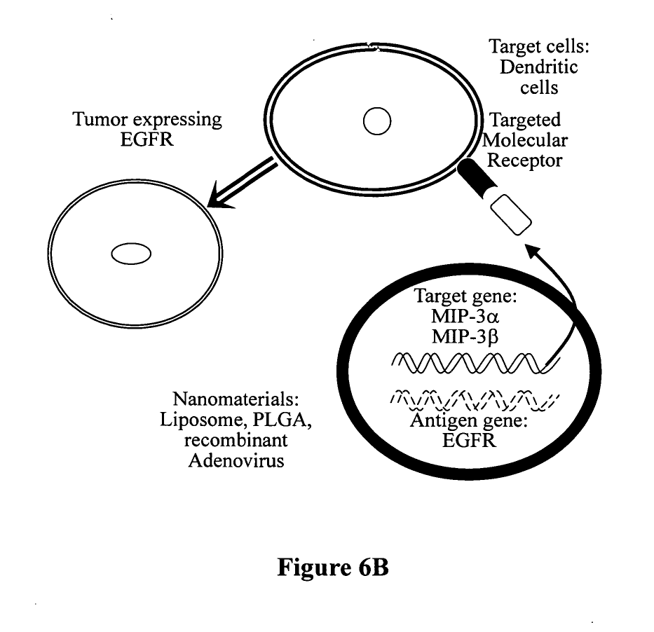

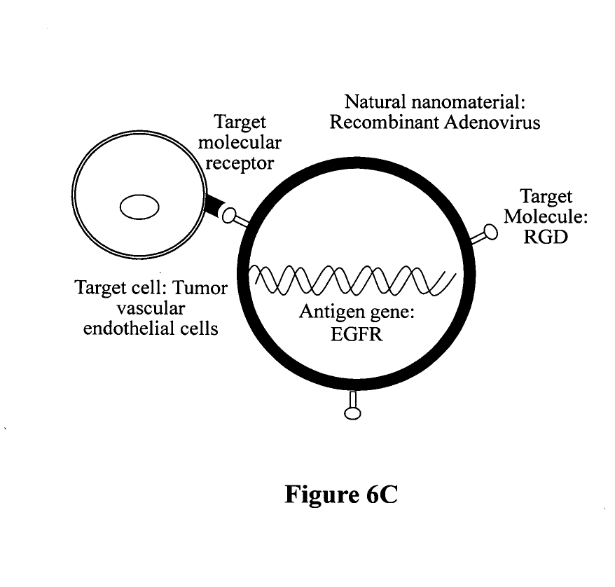

EGFR molecular vaccine targeted with nanoparticles can increase the immunogenicity of EGFR greatly compared to normal EGFR molecular vaccine, and thus enabling EGFR to illicit even stronger anti-tumore immune response. See FIG. 6 for schematic description of the mechanism of action for targeted EGFR molecular vaccine targeted with nanoparticle.

5) Combination of EGFR Molecular Vaccine With Other Immune Response Stimulating Factors

The anti-tumor effect of EGFR molecular vaccine can be enhanced by the combing effect of other immune response stimulating factors. These factors include cytokines such as IL-2, TNF, IFN-γ, and GM-CSF, etc., chemokines such as MIP3α, MIP3β, and IP10, etc., stringent factors such as CEA, HSP70, etc., and various immune co-stimulating factors such as B7, etc. The combinatorial effect of these immune response stimulating factors can realize their effects through gene fusion at the gene level, or fusion protein at the protein level. They can also act through co-transfecting tumor cells, dendritic cells, and vascular endothelia cells at the cellular level.

6. The Anti-Tumor Effect of EGFR Molecular Vaccine

As discussed above, the present invention provides new type of molecular vaccine using tumor associated proteins. As in the specific embodiment of EGFR molecular vaccine, it encompasses protein vaccine, gene vaccine, viral vaccine, and gene-modified vaccine, all made using autologous, xenogeneic, or gene directed evolutionary EGFR molecules. These molecular vaccines possess effective anti-tumor function including preventing tumor formation, inhibit tumor growth, and increase the survival rate of human or animals carrying tumors. Its anti-tumor mechanism of action is: using EGFR molecule as immune cross-reaction antigen, it can break the body's immune tolerance of self-EGFR molecule, and thus inducing the body's self immune cross reaction against the EGFR. These immune reactions include active immune response including humoral and cellular immune response, and passive immune response (adoptive immunity).

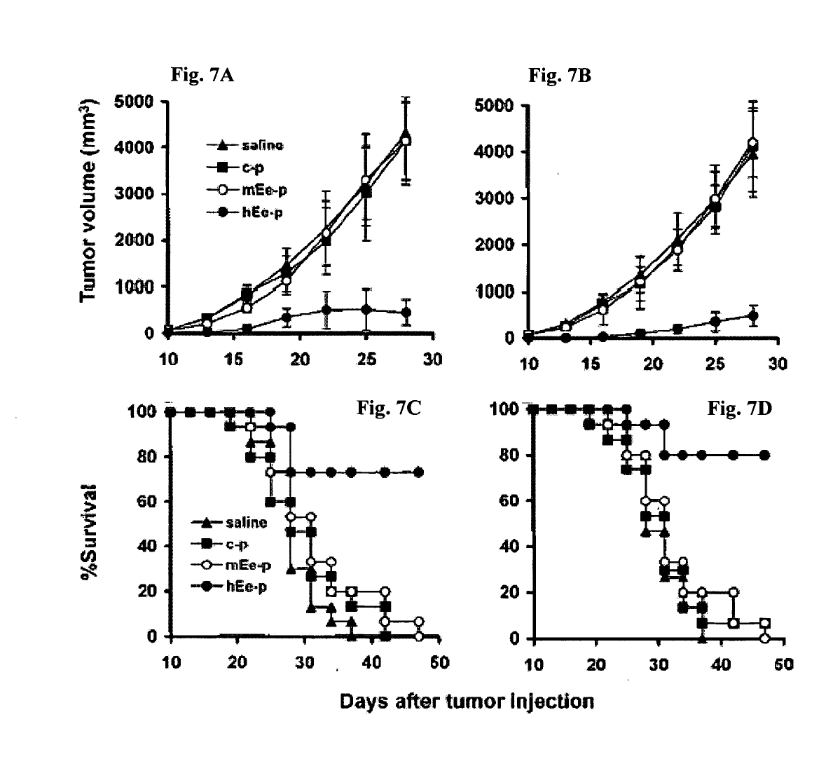

To observe the anti-tumor effect of EGFR recombinant DNA vaccine, mice were randomly divided into groups (15 mice per group), and each group was injected intramuscularly 100 μg EGFR recombinant DNA vaccine, hEe-p, mEe-p, c-p (control plasmid), or Saline, respectively. The injection was done once a week for four weeks continuously. One week after the forth immunization, immunized mice were each implanted with 5×10 5 of LL/2c Lewis lunger cancer cells (FIGS. 7A and C), or MA782/5S mammary cancer cells (FIGS. 7B and D), respectively. It can be seen from the figures that tumors kept growing in mice immunized with mEe-p, c-p, or Saline, while mice immunized with hEe-p exhibited significant protective immunity. In addition, the survival rate of hEe-p immunized mice is significantly higher than those of the mice immunized with mEe-p, c-p, or saline. For example, mice immunized with hEe-p lived longer than 5 months. At 150 days after tumor implantation, mice immunized with LL/2c Lewis lung cancer and with MA782/5S mammary cancer reached survial rate of 60% and 66%, respectively.

It is also provided herein that the protective immunity is dosage-dependent. That is, the immunity obtained with dosage of 150 μg is similar to that of 100 μg of the EGFR DNA vaccine, but there is almost no immunity when the dosage is lowered to 5-15 μg. In addition, Mice bearing EGFR-negative tumors, such as H22 liver cancer, and MMT-06052 mammary cancer exhibited no protective immunity when immunized with hEe-p.

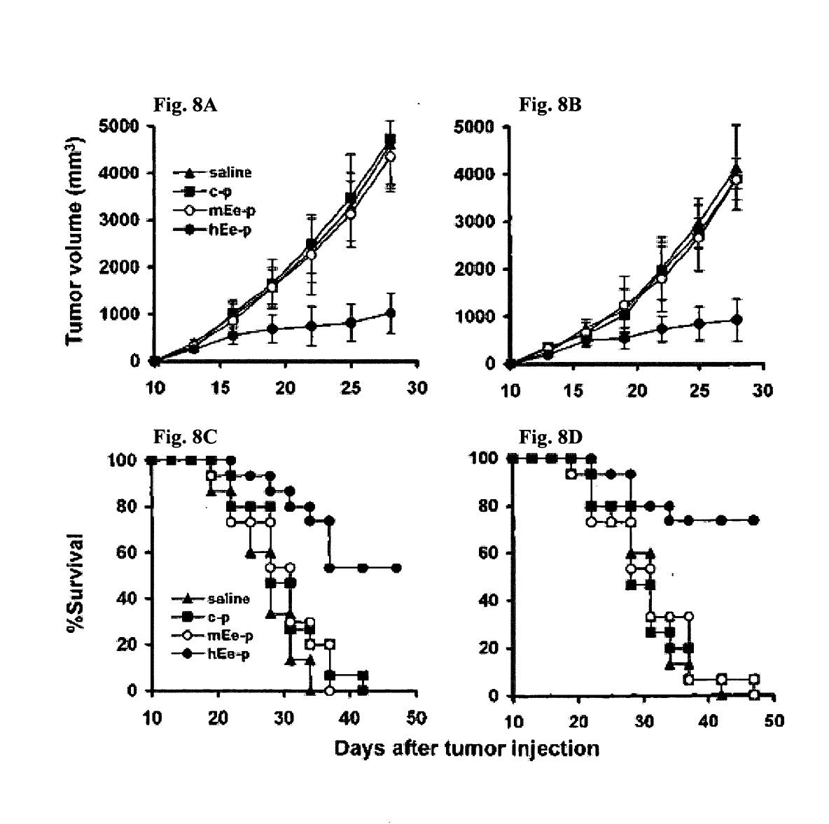

Other than protective immunity, EGFR recombinant DNA vaccine also possesses therapeutic immunity (FIG. 8). As discussed above, mice were randomly divided into groups of 15. They were each injected subcutaneously once weekly for 4 continuous weeks with 1×10 6 LL/2c Lewis Lung cancer cells (FIGS. 8A and C), or MA782/5S mammary cancer cells (FIGS. 8B and D). Five days later, they were injected intramuscularly 100 μg each hEe-p, mEe-p, c-p, or saline once weekly for 4 continuous weeks. As seen from the figures, tumors in mice immunized with mEe-p, c-p, or saline continued to grow, while tumors in mice immunized with hEe-p exhibited significant therapeutic effect. In addition, the survival rate of mice immunized with hEe-p is significantly higher than those immunized with mEe-p, c-p, or saline. Mice immunized with hEe-p lived longer than 5 months. 150 days after tumor implantation, mice carrying LL/2c Lewis cancer and MA782/5S mammary cancer has a survival rate of 40% and 53%, respectively.

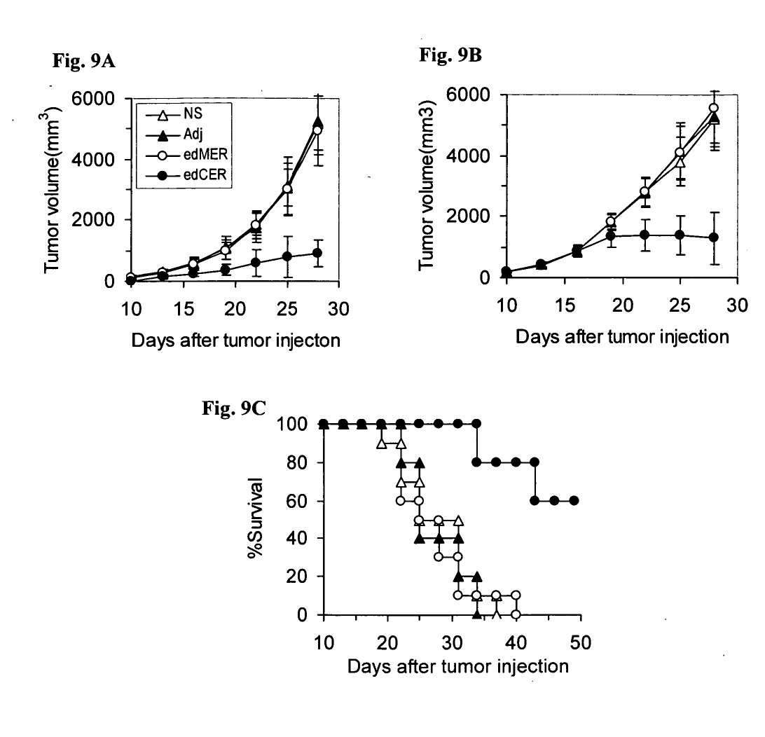

EGFR recombinant protein vaccine has similar protective and therapeutic immunity. See FIG. 9. As described above, 6-8 weeks old female mice which carried C57BL/6 or BALB/c were randomly divided into groups, and tumor models were established for LL/2c Lewis lung cancer, MA782/5S mammary cancer and C26 intestinal cancer in these mice. Tumor-bearing mice were injected subcutaneously EGFR recombinant protein vaccine in the amount of 5-50 μg, or alum adjuvant, or saline 100 μg, once a week for 4 weeks continuously. Mice immunized with recombinant chEGFR protein vaccine exhibited significant anti-tumor protective immunity and therapeutic immunity, and the growth of the LL/2c Lewis lung cancer, and MA782/5S mammary cancer is inhibited, and the life span of mice bearing these two tumors is extended, while the EGFR-native tumor, C26 is not significantly affected. In addition, it can be seen that tumors kept growing at a fast pace in the control groups where mice were immunized with recombinant mEGFR protein vaccine, alum adjuvant, or saline, and the life span of these mice were significantly shortened. Comparing to the control group, the tumor volume (t-test) and the length of survival (log-rank test) in the test groups showed significant difference (p<0.05). FIG. 9 shows the anti-tumor effect of EGFR recombinant protein vaccine in mice bearing MA782/5S mammary cancer.

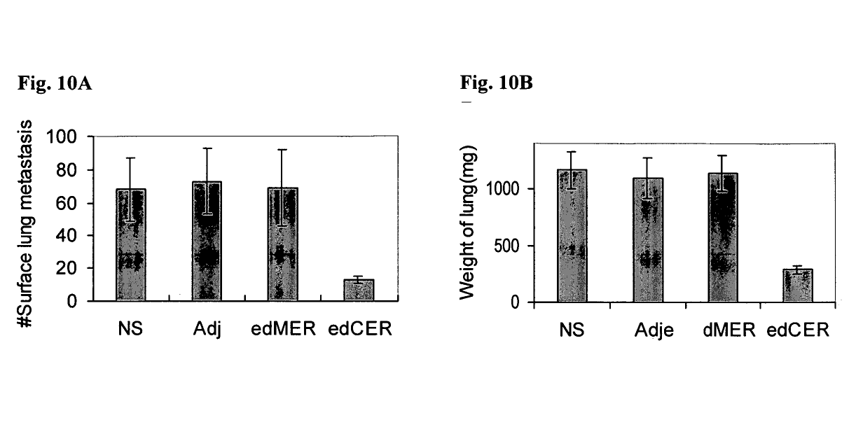

Metastasis is a common cause for tumor progression and for failure of chemothearpy and radiation therapy. The presence of tumor cells in blood and lymph circulation and the formation of microtransferer is key to metastasis. It is disclosed in this invention that recombinant EGFR molecular vaccine has anti-metastasis effect on tumor. See FIG. 10. It is discovered during therapy research of LL/2c Lewis lung cancer metastasis in animal model by injection in the tail vein, tumor-bearing mice immunized with chEGFR protein vaccine has far less metastasis in lung, or to a much lesser degree comparing to control group, whereas tumor-bearing mice in the control groups where they were immunized with recombinant mEGFR protein vaccine, alum adjuvant, or saline showed 100% metastasis, and they showed countless transferer.

In addition, EGFR molecular vaccine can inhibit growth of tumor cells in vitro, and they possess adoptive anti-tumor immunity in vivo. See FIG. 11. The process is described as follows: Mice were immunized with EGFR DNA vaccine, and sera were collected 7 days after the fourth immunization. Serum Immunoglobulin (Ig) were purified using affinity chromatography. Various conentration of Ig (1-1000 mg/ml) were added to 2×10⁵ EGFR-positive tumor cells (A549, LL/2c, MA782/5S) and EGFR-negative tumor cells (H22 and MMT-06052), both kinds of tumor cells being in log-growth stage. The cells and the Ig were then co-cultured for 72 hours, and live cells were examined using Tai Pan Blue method, and the rate of growth inhibition was calculated. The results showed that EGFR-positive tumor cells that were treated with Ig purified from the sera of mice immunized with human EGFR recombinant DNA vaccine hEe-p showed significant growth inhibition, whereas EGFR-negative tumor cells were not affected. See FIG. 11A. As a control, Ig derived from non-immunized normal mice has no effect on either EGFR-positive tumor cells, or EGFR-negative tumor cells. See FIG. 11B.

These purified Ig originated from immunized mice also possess adoptive immunity. The process to study adoptive immunity is as follows: nude mice were injected subcutanously 1×10⁵-1×10⁶ tumor cells. One day later, purified Igs were intravenously injected in the dosage of 10-300 mg/kg, twice a week for three weeks continuously. Results showed that the adoptive transfer of human EGFR recombinant DNA vaccine hEe-p has significant tumor suppression effect. As a control, purified Ig are incubated at 4° C. with fixed EGFR-positive tumor cells or EGFR-native tumor cells for one hour of shaking and mixing in order for the tumor cells to adsorb the Ig. This process is repeated four times. Results showed that Ig from mice immunized with human EGFR DNA vaccine hEe-p were pre-adsorbed by the EGFR-positive tumor cells, such as LL/2c and MA782/5S), and thus lost their anti-tumor effect, while the anti-tumor effect of the Ig still remained after incubating with EGFR-negative tumor cells, such as H22) (FIG. 11C).

Besides the lung cancer and mammary gland cancer as described above, the tumors that can be affected include other solid tumors wherein EGFR is over-expressed, including ovarian cancer, colorectal cancer, prostate cancer, stomach cancer, bladder cancer, head and neck squamocarcinoma and glioma. EGFR molecular vaccine has reliable anti-tumor effect on all cancers mentioned above.

7. Mechanism of Action and Biological Safety of the EGFR Molecular Vaccine

It is well known in modern day immunology that, the mutual recognition between an antigen or antigenic epitope and its corresponding receptor/antibody is not merely specific. In fact, there is a certain degree of plasticity, promiscuity, and degeneracy in this recognition. Thus, it is possible to induce immune cross-reaction against a self-antigen through the mechanism of molecular mimicry in order to break the immuno-tolerance for the self-antigen.

As discussed above, present invention discloses the discovery that tumor specific proteins such as EGFR molecule is a immune cross-reactive antigen. Certain variation of the EGFR molecule can break the body's tolerance for self-EGFR molecule, and induce self cross-reactive immune response against the EGFR molecule. These immune response against EGFR include active immune response (such as cellular immune response and humoral immune response), and passive immune response (such as adoptive immune response).

In order to study the mechanism of the anti-tumor function of EGFR molecular vaccine, we first undertook immune testing for humoral liquid from EGFR immunized mice. The testing used included flow cytometry, Western Blot, immunoprecipitation, in order to determine the existence of anti-EGFR autoantibody in mouse and rabbit serum. We used ELISA to test for the titer and type of antibody in mouse/rabbit immunized sera, we used immunohistochemistry to test the autoantibody in the tumor tissue of immunized mice. We used tumor cell aggregation test, and serum adoptive immune test for examining the function of autoantibody.

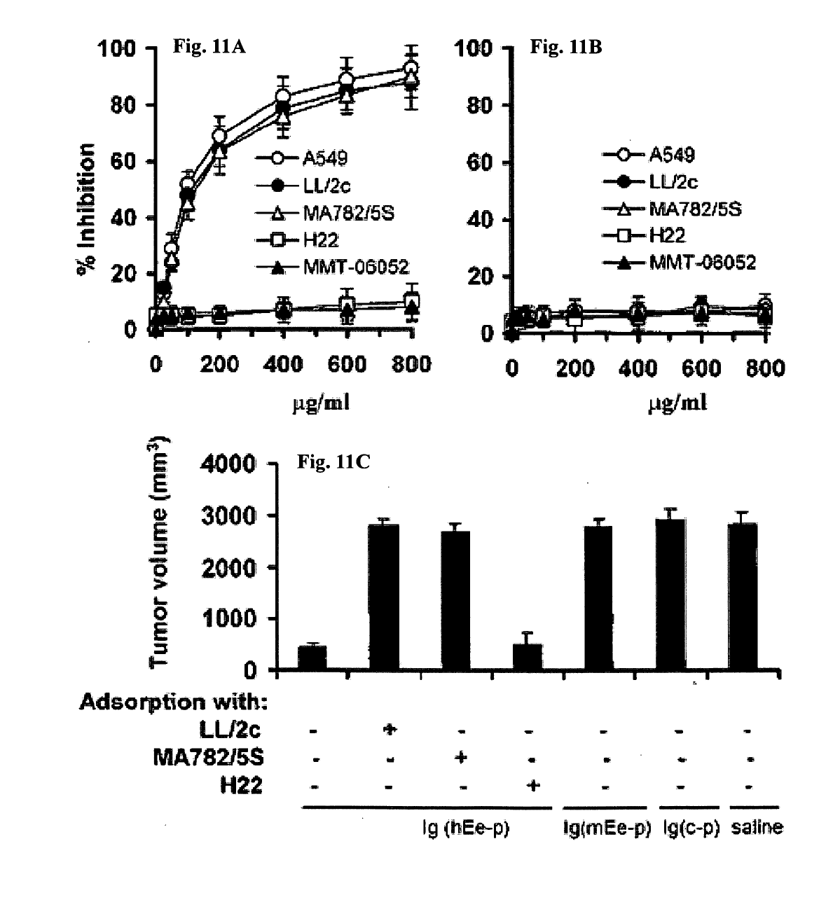

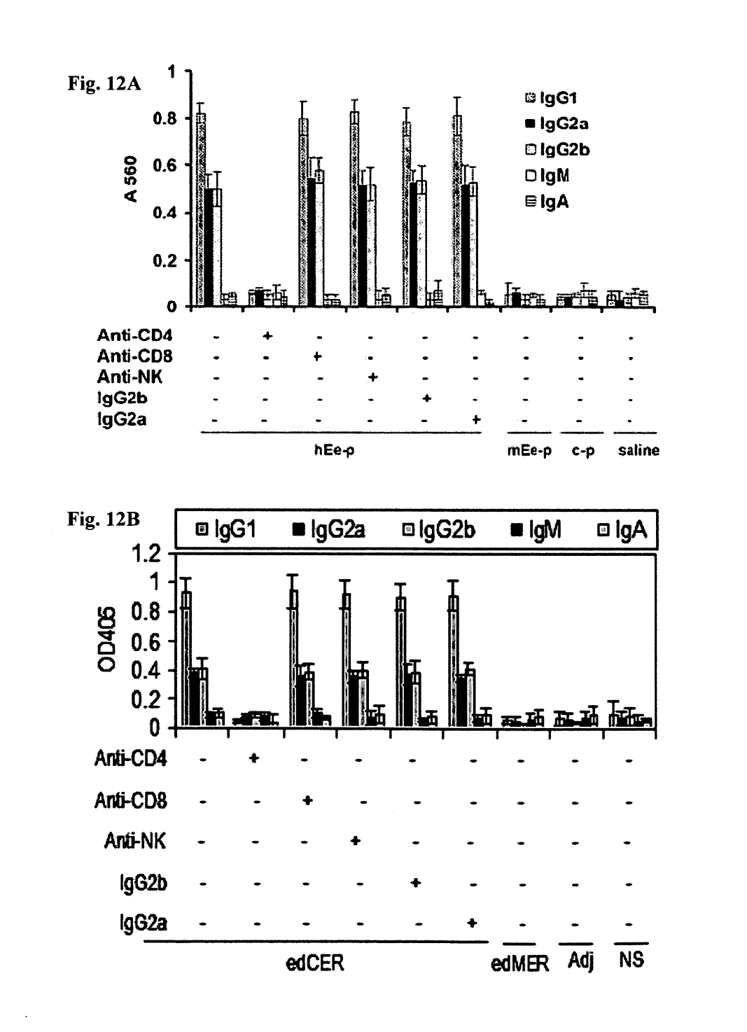

The following experimental protocols were undertaken. Blood was taken from each mouse before and after immunization every week using extraction of blood sample from the mouse tail vein, or after the mouse is sacraficed, sera were collected for use in the next step. Western blot s shows that antibodies induced by recombinant EGFR molecular vaccine can specifically recognize the corresponding antigen (recombinant EGFR protein or EGFR expression in tumor cells), but they can not recognize EGFR-negative cells. At the same time, flow cytometry was used to identify the antibody stereo surface recognition and we found that recombinant EGFR molecular vaccine can stimulate the production of specific antibodies which can in term recognize the EGFR on the surface of tumor cells. Testing for autoantibodies using ELISA showed that mice started to produce anti-mouse autoantibody two weeks after immunized with recombinant EGFR molecular vaccine, reaching a titer of 1:100 to 1:5000. It gradually increased to 1:10000 to 1:500000 at week 4, and can maintain a titer of 1:500 to 1:1000 at week 8. As a comparison, the corresponding control group has no detectable antibody production. FIG. 12 showed the types of antibodies produced by mice immunized with EGFR molecular vaccine (both recombinant DNA vaccine and recombinant Protein vaccine). From the figure, it can be seen that recombinant EGFR molecular vaccine can increase significantly the production of IgG1, IgG2a, and IgG2b , but that the production of IgM and IgA is not increased. Moreover, the production of the antibodies stimulated by recombinant EGFR molecular vaccine can be blocked by anti-CD4 antibody, but not by anti-CD8, anti-NK, or control antibodies. As a control, no specific antibodies were detected in the controls groups of protein vaccine, adjuvant, or saline.

EGFR molecular vaccine can also induce cellular immunity. The tests used to examine cellular immunity in mice immunized with EGFR molecular vaccine include: using Cr⁵¹ release method to measure CTL activities. Using ELISPOT to test cytokine level (mainly for testing serum concentration of IFN-γ, and IL-4), using depletion of immune cell subsets tests to determine T cell types (CD4+ T lymphocyte, CD8+ T lymphocyte, or NK cells, etc.

The protocols are carried out as follows: mice are immunized with recombinant chEGFR vaccine, then spleen monlymphocyte were taken for ELISPOT test, which shows there are a large quantity of antigen specific B cells in the monolymphocyte. Testing in mice immunized with recombinant mEGFR showed the existence of a small amount of antigen specific B cells. But, no statistically significant amount of antigen specific B cells were found in control groups including adjuvant group or blank vector group. In another experiment, spleen T cells were taken from mice immunized with recombinant EGFR molecular vaccine for three weeks, we then use monolymphocyte from health spleen as antigen presenting cells to activate T cells in vitro in the presence of antigen. Results show that mice immunized with chEGFR molecular vaccine contain relatively a larger amount of IFN-γ, and IL-4 producing cells. Spleen T derived from chEGFR molecular vaccine immunized mice cells also produced many IFN-γ, and IL-4 producing cells after being stimulated again in vitro by mEGFR molecular vaccine. Both groups of spleen T cells produce significantly more than T cells that were not stimulated in vitro.

Furthermore, standard Cr⁵¹ tests were used to determine the specific cytocidal effect of spleen T cells immunized by recombinant EGFR molecular vaccine against the relevant tumor cells from human and mice. Results showed that after immunized by chEGFR molecular vaccine, at the ratio of 40:1, mouse spleen T cells exhibited specific cytocidal activity against EGFR positive tumor cells. In the example of LL/2c and MA 782/5S, they are 40.27%, 42.83%, respectively. In contrast, mice spleen T cells immunized with mEGFR exhibited no cytocidal effect on LL/2c and MA 782/5S tumor cells, and on EGFR-negative tumor cells such as C26 cells. At the same time, the cytocidal effect mentioned above can be blocked by corresponding anti-CD8 and anti MHC-I monoclonal antibodies, but was not blocked by anti-CD4 and anti-MHC-II monoclonal antibodies. Spleen T cells from control groups did not show any statistically signifcant cytocidal activity.

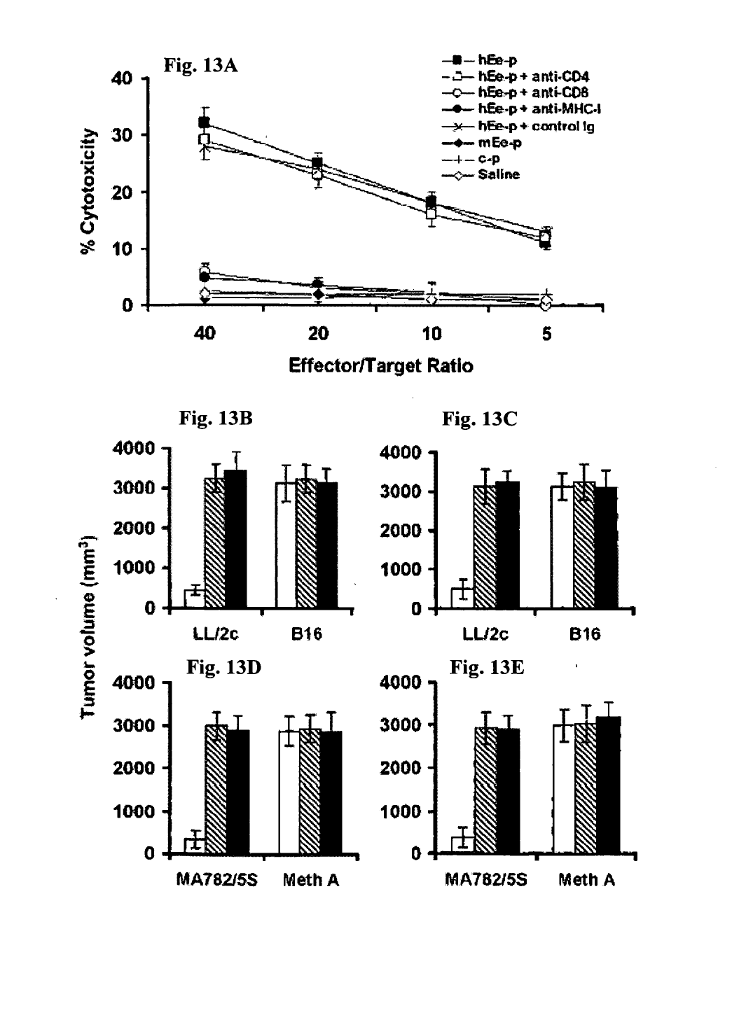

FIG. 13 shows the CTL effect of EGFR recombinant DNA vaccine on immunized mice. It can be seen from this figure that T cells from mice immunized with human EGFR recombinant DNA vaccine hEe-p has higher cytocidal activity against EGFR-positive tumor cells compared to T cells from the control groups. Moreover, this in vitro cytocidal activity can be blocked by anti-CD8 or anti-MHC-I monoclonal antibody, but not by anti-CD4 monoclonal antibody, showing the this cytocidal activity is dependent on MHC-I dependent CD8+ T cells. In addition, activated spleen cells exhibited no increase in NK activity against YAC-1 target cells. Moreover, adoptive transfer of CD4-depleted (CD8+), or CD8-depleted (CD4+) T cells derived from mice immunized with human EGFR recombinant DNA vaccine hEe-p exhibited anti-tumor effect against EGFR-positive tumor. In contrast, its anti-tumor effect is not significant against EGFR-negative tumor cells. No anti-tumor effect was seen from the control group.

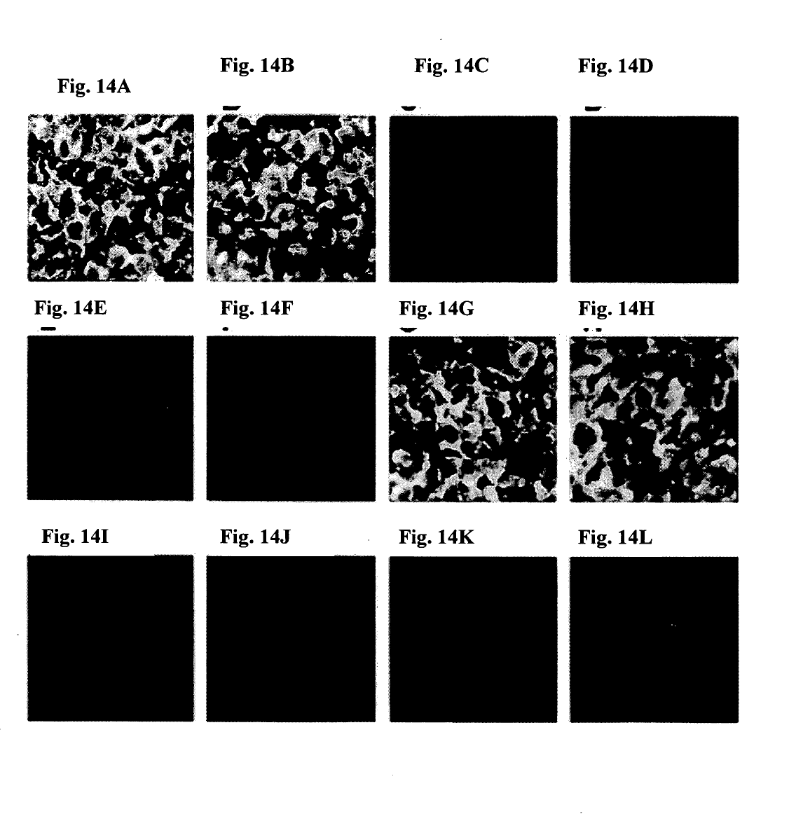

We also observed long-term potential toxic impact on mice immunized with EGFR molecular vaccine. No obvious toxic side effects were observed such as weight loss, skin and hair deterioration, loss of apetitie, reduced life expectancy. Microscopic examination of the liver, lung, spleen, and brain of immunized mice uncovered no pathological changes. Immunofluorescent staining showed no deposition of autoantibody in major organs of the mice. See FIG. 14.

### EXAMPLES

The following examples are included for illustrative purposes only and are not intended to limit the scope of the invention.

### Example One

### EGFR Recombinant DNA Vaccine

GenBank was searched to obtain the cDNA sequences for human, mouse, Chicken, and they are listed herein as SEQ ID NO 1-5,7-9,19, respectively. We designed the following primer based upon the cDNA sequences:

For pORF based plasmids:

Human Primer:
5′GACCATGGAGGAAAAGAAAGTTTGC3′
5′ ACGAATTCTTAGGACGGGATCTTAGGCCCA 3′;
Mouse Primer:
5′GACCATGGAGGAAAAGAAAGTCTGC3′,
5′ ACGAATTCTTAATAGATGGTATCTTTGGC 3′;
Chicken Primer:
5′GACCATGGAGGAGAAGAAAGTTTGTC3′,
5′ ACGAATTCTTAAGATGGAGTTTTGGAGCC 3′;

We use the total RNA from human lung cancer cell line A431, mouse lung cancer cell line LL2, and Chick Embryo to undertake RT-PCR amplification, then collect and purify the amplified EGFR fragment (average 1.9 kb in length) using electrophoresis, and then subclone the PCR products. After confirming the sequences of the PCR subclones through sequencing, we digest them using NcoI and EcoRI, collect the 1.9 kb fragments and purify them, and then insert the fragments into the pORF-MCS vector (from the InvivoGen Corporation) which had been digested with NcoI and EcoRI. We select recombiant plasmids. Candidate recombinant plasmids are confirmed by both restriction enzyme digestion analysis and PCR amplification. They are named pORF-hEGFR, pORF-mEGFR and pORF-chEGFR.

For pcDNA based plasmids:

For the construction of pcDNA-hEGFR, pcDNA-mEGFR and pcDNA-chEGFR, the process is similar to the above described process. In this case, we used pcDNA3.1(+) vector made by InvitroGen, and vary the sequences of the PCR primers somewhat from those primers described in the above paragraph.

Human Primer:
5′GAGCTAGCATGGAGGAAAAGAAAGTTTGC3′,
5′ CACTCGAGTTAGGACGGGATCTTAGGCCCA 3′;
Mouse Primer:
5′GAGCTAGCATGGAGGAAAAGAAAGTCTGC3′,
5′ CACTCGAGTTAATAGATGGTATCTTTGGC 3′;
Chicken Primer:
5′GAGCTAGCATGGAGGAGAAGAAAGTTTGTC3′,
5′ CACTCGAGTTAAGATGGAGTTTTGGAGCC 3′.

We selected recombinant expression plasmids, use restriction enzyme analysis to confirm the presence of the EGFR sequence, and then transfect them into CHO, NIH3T3, Vero cell lines, respectively. We monitor the level of EGFR expression using SDS-PAGE, ELISA, Western Blot techniques. We used alkaline extraction to harvest the recombinant EGFR expression plasmids. Then, we use ultracentrifuge and ultra-filtration to eliminate E. coli toxin contamination, and thus obtaining pure plasmid DNA. These plasmid DNAs can be used as DNA vaccine to immunize subjects, in this case, mice.

Similar to the above process, we can design other primers or probes according to the sequences collected in public databases such as GenBank, the sequences including gene, cDNA, mRNA and amino acid sequences. These sequences can be from a variety of species such as human, mouse, rat, chicken, mackeral, fruit fly, etc, see SEQ ID Nos: 1-14. Using these sequences, we clone and isolate the extracellular domains of EGFR molecules from different biological sources, using PCR, RT-PCR, molecular hybridization techniques, using commercial gene libraries, cDNA libraries (such as ClonTech, StrateGene's gene libraries and cDNA libraries), and cell lines (such as human lung cancer cell line A431, mouse Lewis lung cancer line LL2, etc.), or tissues (such as lung cancer, breast cancer tissues, fruit fly tissue, and chick embryo, etc.). Also, we obtained EGFR molecules possessing strong immunogenicity using selections with the gene-directed evolutionary technique. After confirming the EGFR cDNA extracellular sequences using sequencing, we inserted the EGFR extracellular domain into Eukaryotic expression plasmids such as pcDNA3. 1, pORF-mcs, pBLAST-MCS, pSecTag2, etc.

We selected mice aged 6-8 weeks old, and established various mouse tumor models using routine techniques. We injected 100 μg of EGFR recombinant expression plasmids, once a week, for 4 weeks continuously. We observed the growth of the tumor-bearing mice, and the progression of the tumor. After 8 weeks, the mice were sacrificed, and sera of the immunized mice and organs were collected. We used flowcytometry, ELISA, and Western Blot to test for humoral immune response. To examine cellular response, we used Cr⁵¹, ELISpot methods. To determine toxicity and side effects of the experiments, we used immunohistochemistry. Next, we purified the sera of the immunized mice, used routine methods to establish tumor models in nude mice, and undertake adoptive immunotherapy, and further observe the growth of the tumors.

### Example Two

### EGFR Recombinant Protein Vaccine (Expressed in E. coli)

As in Example One, we obtain the cDNA sequences of EGFR from human, mouse, and chicken from public databases such GenBank, corresponding to SEQ ID NO 1-5,7-9,19, respectively. Based upon these sequences, we designed the following primers:

Human Primer:
5′GACCATGGAGGAAAAGAAAGTTTGC3′,
5′ACAGATCTAGGACGGGATCTTAGGCCCA3′.
Mouse Primer:
5′GACCATGGAGGAAAAGAAAGTCTGC3′,
5′ACAGATCTATAGATGGTATCTTTGGC3′.
Chicken Primer:
5′GACCATGGAGGAGAAGAAAGTTTGTC3′,
5′ACAGATCTAGATGGAGTTTTGGAGCC3′.

We use pORF-hEGFR, pORF-mEGFR and pORF-chEGFR as template for PCR amplification, then collect and purify the amplified EGFR fragment (average 1.9 kb in length) using electrophoresis, and then subclone the PCR products. After confirming the sequences of the PCR subclones through sequencing, we digest them using NcoI and BglII, collect the 1.9 kb fragments and purify them, and then insert the fragments into the pQE60 vector (from the Qiagen Corporation) which had been digested with two restriction enzymes NcoI and BglII. We select recombiant plasmids. Candidate recombinant plasmids are confirmed by both restriction enzyme digestion analysis and PCR amplification. They are named pQE-hEGFR, pQE-mEGFR and pQE-chEGFR.

Similarly, depending upon different prokaryotic expression vectors such as pET32, pLLp, pSE420, different primers can be designed and used to construct other EGFR recombinant prokaryotic expression plasmids.

Similar to Example One, we cloned the extracellular domain of various EGFR molecules, and then insert the domain into the prokaryotic expression plasmids such as pQE, pET32, pLLp, etc. After selecting recombinant expression plasmids, we confirmed the sequences of the domain using restriction enzyme digestion. We then transformed these recombinant plasmids into E. coli hosts such as E. coli TOP10F′, E. coli BL21(DE3)pLys, E. coli M15, etc., and then observed and tested the level of expression of EGFR molecule using techniques such as SDS-PAGE, ELISA, Western Blot, etc. From these testing, we determined the optimal E. coli host for expression the recombinant plasmids. We then used confirmed EGFR expression plasmids to transform the optimal E. coli host cells, and then established stable transformants and various levels of seeding warehouse libraries. We cultured the recombinant bacterial cells in shaking incubators, or alternatively, we fermented the bacteria using standard methods. We harvested the recombinant bacteria using low temperature centrifuge. Then we suspended the bacteria in PBS, and then broke the cells using ultrasound method. We used ionic exchange chromatography and affinity chromatography to isolate and purify the recombinant EGFR proteins. The purified EGFR proteins can be used as protein vaccine to immunize subject mice.

As in Example One, we selected mice aged 6-8 weeks old, and established various mouse tumor models using routine techniques. We injected 5-50 μg of EGFR recombinant protein, once a week, for 4 weeks continuously. We observed the growth of the tumor-carrying mice, and the development of the tumor. After 8 weeks, the mice were sacrificed, and sera of the immunized mice and organs were collected. We used flowcytometry, ELISA, and Western Blot to test for humoral immune response. To examine cellular response, we used Cr⁵¹ ELISpot methods. To determine toxicity and side effects of the experiments, we used immunohistochemistry. Next, we purified the sera of the immunized mice, used routine methods to establish tumor models in nude mice, and undertake adoptive immunotherapy, and further observe the growth of the tumors.

### Example Three

### EGFR Recombinant Protein Vaccine (Expressed in Yeast Pichia pastoris)

Again, as described in the previous sections, we obtain the cDNA sequences of EGFR from human, mouse, and chicken from public databases such GenBank, corresponding to SEQ ID NO 1-5,7-9,19, respectively. Based upon these sequences, we designed the following primers.

Human Primer:
5′ATACTCGAGAAAAGAGAGCTGGAGGAAAAGAAAG3′
5′GCTCTAGAATGGCACAGGTGGCACA3′;
Mouse Primer:
5′ATGCTCGAGAAAAGAGAGTTGGAGGAAAAGAAAGTC3′,
5′AAGCGGCCGCCATAGATGGTATCTTTG3′;
Chicken Primer:
5′ATACTCGAGAAAAGAGAGGTGGAGGAGAAGAAAG3′,
5′CGTCTAGAAGATGGAGTTTTGGAG3′.

We use pORF-hEGFR, pORF-mEGFR and pORF-chEGFR as template for PCR amplification, then collect and purify the amplified EGFR fragment (average 1.9 kb in length) using electrophoresis, and then subclone the PCR products. After confirming the sequences of the PCR subclones through sequencing, we digest them using two restriction enzymes XhoI and XbaI (in the case of plasmids containing mouse EGFR, we use XhoI and NotI double enzyme digestion), collect the 1.9 kb fragments and purify them, and then insert the fragments into pPICZ A vector (from Invitrogen Corporation) which had been digested with two restriction enzymes XhoI and XbaI (in the case of plasmids containing mouse EGFR, we use XhoI and NotI double enzyme digestion). We transformed E. coli with these plasmid preparations, and select recombiant plasmids. Candidate recombinant plasmids are confirmed by both restriction enzyme digestion analysis and PCR amplification. They are named yeast expression plasmids: pYE-hEGFR, pYE-mEGFR and pYE-chEGFR.

After digesting these yeast expression plasmids with PmeI to linerize them, we use electric perforation method to transform yeast cell lines X33, or GS115. We use Zeocin resistance to select stable transformants. We use MMH (Minimal Methanol with histidine, MMH) and MDH (Minimal Dextrose with histidine, MDH) agar plate) to determine and selecte Mut+ transformants. We select 6-10 Mut+ transformants for small-scale expression, and then use SDS-PAGE, Western Blot, ELISA, etc. to confirm the expressed recombinant protein. We select the Mut+ transformants with the highest expression efficienty, and culture them at a large scale so as to establish yeast expression seed libraries. We use large flasks to culture or ferment the recombinant yeast cell lines, collect the yeast pellets using low temperature centrifuge. After resuspend the pellets n PBS solution, we use ultrasound to break the cells. Then, we used Ion Exchange Chromatography and affinity chromatography to isolate and purify the recombinant EGFR protein. The recombinant EGFR protein derived as such can be used as protein vaccine to immunize subjects. Similarly, yeast recombinant expression plasmids made with EGFR can be produced with other yeast expression systems.

As in Example Two, we selected mice aged 6-8 weeks old, and established various mouse tumor models using routine techniques. We injected 5-50 μg EGFR recombinant protein, once a week, for 4 weeks continuously. We observed the growth of the tumor-carrying mice, and the development of the tumor. After 8 weeks, the mice were sacrificed, and sera of the immunized mice and organs were collected. We used flowcytometry, ELISA, and Western Blot to test for humoral immune response. To examine cellular response, we used Cr⁵¹, ELISpot methods. To determine toxicity and side effects of the experiments, we used immunohistochemistry. Next, we purified the sera of the immunized mice, used routine methods to establish tumor models in nude mice, and undertake adoptive immunotherapy, and further observe the growth of the tumors.

### Example Four

### EGFR Recombinant Adenovirus Vaccine

As in the previous examples, we obtained the cDNA sequences of EGFR from human, mouse, and chicken from public databases such GenBank, corresponding to SEQ ID NO 1-5,7-9,19, respectively. Based upon these sequences, we designed the following primers:

Human Primer:
5′GAAGATCTATGGAGGAAAAGAAAGTTTGC3′,
5′ACGATATCTTAAGGACGGGATCTTAGGCCCA3′;
Mouse Primer:
5′GAAGATCTATGGAGGAAAAGAAAGTCTGC3′,
5′ACGATATCTTAATAGATGGTATCTTTGGC3′;
Chicken Primer:
5′GAAGATCTATGGAGGAGAAGAAAGTTTGTC3′,
5′ACGATATCTTAAGATGGAGTTTTGGAGCC3′;

We use pORF-hEGFR, pORF-mEGFR and pORF-chEGFR as template for PCR amplification, then collect and purify the amplified EGFR fragment (average 1.9 kb in length) using electrophoresis, and then subclone the PCR products. After confirming the sequences of the PCR subclones through sequencing, we digest them using two restriction enzymes BglII and EcoRV double enzyme digestion, collect the 1.9 kb fragments and purify them, and then insert the fragments into pShuttle-CMV vector (made by Quantum Biotechnologies) which was pre-digested with BglII and EcoRV. We select recombiant plasmids. The resulting candidate recombinant plasmids are confirmed by both restriction enzyme digestion analysis and PCR amplification. They are named Adenovirus shuttle expression plasmids: pShuttle-hEGFR, pShuttle-mEGFR and pShuttle-chEGFR. See FIG. 2D for plasmid maps of these Adenovirus shuttle expression plasmids.

We then take the various Adenovirus shuttle expression plasmids as described before, digest them with PmeI enzyme, and co-transform E. coli BJ5183 cells with backbone vector containing the Adenovirus genome pAdEasy-1 or pAdEast-2. The resulting recombinant Adenovirus vector plasmids are named pAd-hEGFR, pAd-mEGFR and pAd-chEGFR. These Adenovirus vector plasmids are digested with PacI enzyme, and then using the Calcium-phosphate-DNA coprecipitation method, they are transfected into the Adenovirus packaging cell line 293 cells. The resulting recombinant Adenoviruses are called Ad-hEGFR, Ad-mEGFR and Ad-chEGFR. PCR, Western blot and other methods are used to confirm that the EGFR gene has been indeed integrated into the Adenovirus vector, and that EGFR has been correctly, and efficiently expressed in Eukaryotic cells.

Depending upon the difference of the Adenovirus Genome, the recombinant Adenovirus vaccine can be classified into two groups: the first group is called Generation I of EGFR Recombinant Adenovirus, resulting from the recombination between Adenovirus Shuttle expression plasmid pShuttle-EGFR and AdEasy-1 recombinant, thus named Ad-hEGFR I, Ad-mEGFR I and Ad-chEGFR I. The second group is called Generation II of EGFR Recombinant Adenovirus, resulting from the recombination between Adenovirus Shuttle expression plasmid pShuttle-EGFR and AdEasy-2 recombinant, thus named Ad-hEGFR II, Ad-mEGFR II and Ad-chEGFR II.

We used ultracentrifuge to collect large quantity of the recombinant adenovirus Ad-EGFR, and then tested the titer (pfu) of each batch of the recombinant virus using upper layer agarose method, and TCID50 method. We then used 293 cells to produce large amount of confirmed EGFR adenovirus vaccine. We further isolated and purified the recombinant virus using ultracentrifuge and ultrafiltration. The purified EGFR recombinant adenovirus can be used a vaccine to immunize subject animals.

Similar to what's described in the previous examples, we selected mice aged 6-8 weeks old, and established various mouse tumor models using routine techniques. We injected 1×10⁹ PFU EGFR recombinant adenovirus in each mouse, once a week, for 4 weeks continuously. We observed the growth of the tumor-carrying mice, and the development of the tumor. After 8 weeks, the mice were sacrificed, and sera of the immunized mice and organs were collected. We used flowcytometry, ELISA, and Western Blot to test for humoral immune response. To examine cellular response, we used Cr⁵¹, ELISpot methods. To determine toxicity and side effects of the experiments, we used immunohistochemistry. Next, we purified the sera of the immunized mice, used routine methods to establish tumor models in nude mice, and undertake adoptive immunotherapy, and further observe the growth of the tumors.

### Example Five

### EGFR Recombinant Lentivirus Vaccine

As an illustration of the process of making EGFR Recombinant Lentivirus, we use the ViraPower Lentiviral Gateway Expression Kit made by Invitrogen Corporation. The detailed process is as follows: we first search public database such as GenBank to obatin cDNA sequences of EGFR molecule of human, mouse and chicken. Their sequences are listed as SEQ ID NO 1-5, 7-9, 19, respectively.

We then designed the following PCR primers:

Human Primer:
5′GACCATGGAGGAAAAGAAAGTTTGC3′,
5′ACGATATCAGGACGGGATCTTAGGCCCA3′;
Mouse Primer:
5′GACCATGGAGGAAAAGAAAGTCTGC3′,
5′ACGATATCATAGATGGTATCTTTGGC3′;
Chicken Primer:
5′GACCATGGAGGAGAAGAAAGTTTGTC3′,
5′ACGATATCAGATGGAGTTTTGGAGCC3′.

We use pORF-hEGFR, pORF-mEGFR and pORF-chEGFR as template for PCR amplification, then collect and purify the amplified EGFR fragment (average 1.9 kb in length) using electrophoresis, and then subclone the PCR products. After confirming the sequences of the PCR subclones through sequencing, we digest them using two restriction enzymes NcoI and EcoRV double enzyme digestion, collect the 1.9 kb fragments and purify them, and then insert the fragments into pENTR11 vector ( made by Invitrogen) which was pre-digested with NcoI and EcoRV. We select recombiant plasmids. The resulting candidate recombinant plasmids are confirmed by both restriction enzyme digestion analysis and PCR amplification. They are named as pENTR-hEGFR, pENTR-mEGFR and pENTR-chEGFR. See FIG. 2E for plasmid maps of these Lentivirus expression plasmids.

We then take the various Lentivirus expression plasmids as described before, co-transform E. coli DH5 cells together with backbone vector containing the Lentivirus genome pLenti6/V5-DEST. The resulting recombinant Lentivirus vector plasmids are named pLenti-hEGFR, pLenti-mEGFR and pLenti-chEGFR. See also FIG. 2E. We then mix these Recombinant Lentivirus vector plasmids with packaging mix, the ViraPower Packaging Mix, and then using the Calcium-phosphate-DNA coprecipitation method, the Recombinant Lentivirus vector plasmids are transfected into the Lentivirus packaging cell line 293FT cells. The resulting recombinant Lentivirus are called Lenti-hEGFR, Lenti-mEGFR and Lenti-chEGFR. PCR, Western blot and other methods are used to confirm that the EGFR gene has been indeed integrated into the Lentivirus vector, and that EGFR has been correctly, and efficiently expressed in Eukaryotic cells.

We used ultracentrifuge to collect large quantity of the recombinant adenovirus Ad-EGFR, and then tested the titer (pfu) of each batch of the recombinant virus using upper layer agarose method, and TCID50 method. We then used 293 cells to produce large amount of confirmed EGFR adenovirus vaccine. We further isolated and purified the recombinant virus using ultracentrifuge and ultrafiltration. The purified EGFR recombinant adenovirus can be used a vaccine to immunize subject animals.

Similar to what's described in the previous examples, we selected mice aged 6-8 weeks old, and established various mouse tumor models using routine techniques. We injected 1×10⁹ PFU EGFR recombinant Lentivirus in each mouse, once a week, for 4 weeks continuously. We observed the growth of the tumor-carrying mice, and the development of the tumor. After 8 weeks, the mice were sacrificed, and sera of the immunized mice and organs were collected. We used flowcytometry, ELISA, and Western Blot to test for humoral immune response. To examine cellular response, we used Cr⁵¹, ELISpot methods. To determine toxicity and side effects of the experiments, we used immunohistochemistry. Next, we purified the sera of the immunized mice, used routine methods to establish tumor models in nude mice, and undertake adoptive immunotherapy, and further observe the growth of the tumors.

### Example Six

### Mannan-Modified Recombinant Adenovirus EGFR Vaccine

The following procedure applies to the preparation of Mannan-modified Recombinant Adenovirus EGFR vaccine: we first use standard protocol to obtain and amplify EGFR Recombinant Adenovirus (either Generation I or II), and then use chromatography or ultracentrifuge to purify the recombinant adenovirus. Next, we dissolve 70 mg Mannan from Sigma into 5 ml 0.1M phosphate buffer (pH6.0) to reach a final concentration of 14 mg/ml, and then add 45 ml 0.01M Sodium Periodate solution, and mix and oxidize at 4° C. for 60 minutes. After that, we add 10 μl glycol, and incubate for 30 minutes at 4° C., resulting in Oxidative Mannan (Ox-M) mixture.

We then load the Ox-M mixture onto Sephadex-G25 columns previously balanced with bicarbonate buffer (pH6.0-9.0) and perform chromatography, with Ox-M being eluted into 2 ml sized empty vessel. After that, we mix the purified Ox-M with 1×10¹⁴ Recombinant Adenovirus particles at room temperature overnight, obtaining the needed Ox-M-Adenovirus. We then add 1 mg/ml Sodium Borohydride to the Ox-M-Adenovirus, leave at room temperature for 3 hours, forming Reductive Mannan Adenovirus (Red-M-Adenovirus). Both Ox-M-Adenovirus and Red-M-Adenovirus are desalted by ultrafiltration, and condensed, filtering out bacteria. They are stored in small test tubes, and preserved at −80° C. The Mannan-modified Recombinant EGFR Adenovirus can be used as vaccine to immunize a subject.

Similar to what's described in the previous examples, we selected mice aged 6-8 weeks old, and established various mouse tumor models using routine techniques. We injected 1×10¹⁰ PFU EGFR recombinant Lentivirus in each mouse, once a week, for 4 weeks continuously. We observed the growth of the tumor-carrying mice, and the development of the tumor. After 8 weeks, the mice were sacrificed, and sera of the immunized mice and organs were collected. We used flowcytometry, ELISA, and Western Blot to test for humoral immune response. To examine cellular response, we used Cr⁵¹, ELISpot methods. To determine toxicity and side effects of the experiments, we used immunohistochemistry. Next, we purified the sera of the immunized mice, used routine methods to establish tumor models in nude mice, and undertake adoptive immunotherapy, and further observe the growth of the tumors.

### Example Seven

### RGD-Modified Recombinant Adenovirus EGFR Vaccine

In the present invention, we utilize the AdEasy system to construct the RDG modified Adenovirus Recombinant EGFR vaccine. The detailed process is shown step by step in FIG. 5.

The detailed process is as follows: we digest the Adenovirus backbone plasmid pAdEasy-1 and pAdEasy-2 with restriction enzyme SpeI (Sp), and then use T4 DNA Polymerase to fill in the ends (filling, f) so as to make them blunt, and then digest the filled-in product with PacI (P), and recover the 6211 bp and 3579 bp fragments using electrophoresis, and name them AdFiber I/Sp/f/P and AdFiber II/Sp/f/P, respectively. These fragments contain the intact Adenovirus fiber protein gene. Separately, prepare the pSuttle vector by digesting it with BamHI first, and then fill in with T4 DNA Polymerase, and then digest with PacI. After such BamHI/filling/PacI-digestion treatment, the vector is ready to be inserted with the AdFiber I/Sp/f/P and AdFiber II/Sp/f/P fragments. The resulting plasmids are named pSh-AdFiber I and pSh-AdFiber II, respectively.

We then digest pSh-AdFiber I with NheI enzyme, fill in with T4 DNA polymerase, and digest again with KpnI enzyme (NheI/filling/KpnI), recover, using electrophoresis, the 2090 bp fragment called AdFiber I/Nh/f/K; insert this fragment into a pUC18 vector which had been pre-digested with SmaI and KpnI double enzyme digestion, resulting in the recombinant plasmid named pUC-AdFiber I.

On the other hand, pSh-AdFiber II is digested with AvrII enzyme, then filled in with T4 DNA polymerase, and then digested with HindIII (AvrII/filling/HindIII), using electrophoresis, recover the 838 bp fragment, called AdFiber I/A/f/H. Insert this fragment into a pUC vector which had been previously digested with SmaI and HindIII double enzyme digestion, resulting in new plasmids named pUC-AdFiber II.

Next, we designed a series of PCR primers so as to use pUC-AdFiber I and pUC-AdFiber II as templates to amplify the Adenovirus knob, (Ad-knob) gene sequences. The primers used are, respectively:

F1 (5′GAAAGCTAGCCCTGCAAACATCA3′),
R1 (5′ACTCCCGGGAGTTGTGTCTCCTGTTTCCTG3′),
F2 (5′ACTCCCGGGAGTGCATACTCTATGTCA3′),
R2 (5′TATGGTACCGGGAGGTGGTGA3′),
F3 (5′AACCTAGGGAGGTTAACCTAAGCACTG3′),
and
R3 (5′CTCAAGCTTTTTGGAATTGTTTGA3′).

Using primer F1-R1, F2-R2, F3-R1 and F2-R3, respectively, for the first round PCT, we obtain products PCR1, PCR2, PCR3 and PCR4. Again, using F1-R2 and F3-R3 as primers, and using the amplified products from the first round, PCR1 and PCR2, PCR3 and PCR4 as templates, we undertake the second round of PCR amplification, resulting in PCR products PCR1-PCR2 (PCR I), PCR3-PCR4 (PCR II) . We take the PCR I and PCR II from the amplification in the second round, insert them into pBR322 vector that had been previously cut with EcoRV. The resulting recombinant plasmids are named pBR-PCR I and pBR-PCR II.

The sequence of the RGD-4C duplex is as follows:

5′TGTGACTGCCGCGGAGACTGTTTCTGC3′,
3′ACACTGACGGCGCCTCTGACAAAGACG5′;

We insert the RGD-4C into the pBR-PCR I and pBR-PCR II vectors, where the vector were previously digested with SmaI. The resulting recombinant plasmids are named pBR-PCR/RGD I and pBR-PCR/RGD II. The sequences of the recombinant plasmids are confirmed using sequencing. Cut pBR-PCR/RGD I with NheI/KpnI double digestion , using electrophoresis, rcover the PCR/RGD I fragment. Insert the fragment into the pUC-AdFiber I vector which had been double digested using NheI/KpnI. The resulting recombinant plasmids are called pUC-AdFiber-RGD I.

Similarly, we use AvrII/HindIII to double digest pBR-PCR/RGD II, and using electrophoresis, recover the PCR/RGD II fragment. Again, insert the fragment into pUC-AdFiber II vectors previously digested with double enzume AvrII/HindIII, the resulting plasmid is named pUC-AdFiber-RGD II.

Afterwards, we use SpeI/PacI double enzyme to digest pUC-AdFiber-RGD I and pUC-AdFiber-RGD II vector, and using electrophoresis, recover the AdFiber-RGD I, and AdFiber-RGD II fragments. Insert the fragment into pAdEasy-1, pAdEasy-1 both of which were previously digested with SpeI/PacI, the resulting recombinant plasmids are called pAdEasy-RGD I, and pAdEasy-RGD II, respectively.

Next, we first take pShuttle-hEGFR, pShuttle-mEGFR and pShuttle-chEGFR as described above, and linerize them with PmeI. After that, we co-transform the E. coli BJ5183 cells with these Shuttle-EGFR plasmids together with pAdEasy-RGD I, and pAdEasy-RGD II, respectively. The resulting recombinant plasmids are named Adenovirus plasmids pAd-RGD-EGFR I, and pAd-RGD-EGFR II.

We used the Adenovirus plasmid pAd-RGD-EGFR I to transfect 394 cells, resulting in recombinant Adenovirus named Ad-RGD-EGFR I. Similarly, we transfect Adenovirus plasmid pAd-RGD-EGFR II to 293E4pIX cells, resulting in recombinant Adenovirus named Ad-RGD-EGFR II. After purification, Ad-RGD-EGFR I and Ad-RGD-EGFR II can be used as vaccine to immunize subjects, and more importantly, these vaccines have specificity targeting tumor vascular endothelial cells.

### Example Eight

### Pharmacological Studies of Recombinant EGFR Molecular Vaccine

It is important to obtain pharmacological studies relating to the anti-tumor effect of recombinant EGFR molecular vaccine. Studies can be performed for this purpose in the present invention include: ordinary tumor-bearing mice (including protective immunity study, therapeutic immunity study, and metastasis model study), rabbit immunological test, monkey immunological test, nude mice adoptive immunological test, dosage-dependency test, and in vitro cellular test, etc. In this example, we describe the ordinary tumor-bearing mice test, using lung cancer as a model. Other tests can be performed similarly.

1. Protective Immunity of Tumor-Bearing Mice:

6-8 weeks old C57BL/6 mice are randomly divided into groups. They are injected EGFR molecular vaccine in both hind legs intramuscularly, once a week for four weeks continuously. At the 5^{th} week, mice were are challenged with LL/2c tumor cells in the concentration of 1×10⁶. Blood is taken from tail vein or from sacraficing the mice at week 0, 1, 3, 5 after initial immunization. After centrifuge (5000 RPM for 3 minutes), sera is collected and stored at −20° C. for future use.

2. Therapeutic Immunity of Tumor-Bearing Mice:

6-8 weeks old C57BL/6 mice are randomly divided into groups. They are challenged with LL/2 cancer cells at a concentration of 1×10⁶. Four days after tumor introduction, they are randomly divided into groups, and immunized with EGFR molecular vaccine by injection intramuscularly in the hind legs, once a week for four weeks continuously. At week 0, 2. 4, and 6 after initial immunization, blood is taken from tail vein or from sacraficing the mice. After centrifuge (5000 RPM for 3 minutes), sera is collected and stored at −20° C. for future use. Record tumor weight, volume and survial curves.

3. Metastasis Model Test of Tumor-Bearing Mice:

6-8 weeks old C57BL/6 female mice are randomly divided into groups. They are injected EGFR molecular vaccine in both hind legs intramuscularly, once a week for four weeks continuously. After two weeks, LL/2 tumor cells at log growth period are selected, and injected intramuscularly into the hind legs of the mice, at a concentration of 2×10⁵ per mouse. Contiue the injection mentioned above. After four weeks, mice are killed, and their lungs are weighed. After examination for tumor transferer in the lung, the lungs are fixated with 10% neutrally buffered formaldehyde.

It is understood that the examples and embodiments described herein are for illustrative purposes only and that various modifications or changes in light thereof will be suggested to persons skilled in the art and are to be included within the spirit and purview of this application and scope of the appended claims.

1. A vaccine comprising a molecular homolog having sufficient structural similarity to a tumor specific protein endogenously expressed in a tumor such that the molecular homolog is capable of inducing an immune response to the tumor specific protein in a subject bearing the tumor.

2. The vaccine of claim 1 wherein the molecular homolog is a xenogeneic homolog of the tumor specific protein.

3. The vaccine of claim 1 wherein the molecular homolog is generated using genetic engineering.

4. The vaccine of claim 1 wherein the molecular homolog is a DNA molecule.

5. The vaccine of claim 1 wherein the molecular homolog is a protein molecule.

6. The vaccine of claim 1 wherein the molecular homolog is attached to a virus as a carrier.

7. The vaccine of claim 6 wherein the virus is either an Adenovirus or a Lentivirus.

8. The vaccine of claim 1 wherein the tumor specific protein is a tumor receptor.

9. The vaccine of claim 8 wherein the tumor receptor is an epidermal growth factor receptor (EGFR).

10. The vaccine of claim 9 wherein the structural similarity between the molecular homolog and EGFR ranges from 30-95%.

11. The vaccine of claim I wherein the molecular homolog is modified by attaching thereto a nanoparticle in order to enhance the target specificity of the vaccine.

12. The vaccine of claim 11 wherein the nanoparticle is an Adenovirus.

13. The vaccine of claim 12 wherein the Adenovirus is modified by the peptide RGD.

14. The vaccine of claim 1 wherein the subject is an animal.

15. The vaccine of claim 1 wherein the subject is a human.

16. The vaccine of claim 9 wherein the tumor is selected from the group consisting of mammary cancer, lung cancer, melanoma, hepatocarcinoma, fibrosarcoma, ovarian cancer, colorectal cancer, prostate cancer, stomach cancer, bladder cancer, head and neck squamocarcinoma, and glioma.

17. A vaccine suitable for administering to a human or animal to inhibit growth or formation of a tumor comprising a molecular homolog having sufficient structural similarity to a tumor specific protein endogenously expressed in the tumor such that the molecular homolog is capable of inducing an immune response to the tumor specific protein in the subject.

18. The vaccine of claim 17, further comprising a pharmaceutically acceptable carrier.

19. The vaccine of claim 17, further comprising a nanoparticle which provides targeted modification of the vaccine.

20. The vaccine of claim 19 wherein the nanoparticle has a diameter under 500 nm.

21. The vaccine of claim 20 wherein the nanoparticle has a diameter between 200 nm-500 nm.

22. The vaccine of claim 20 wherein the nanoparticle has a diameter between 100-200 nm.

23. The vaccine of claim 20 wherein the nanoparticle has a diameter between 50-100 nm.

24. The vaccine of claim 19 wherein the nanoparticle is selected from the group consisting of liposome, PLGA, and Mannan-modified Adenovirus.

25. A cell capable of expressing the vaccine of claim 1.

26. A commensal bacteria cell transformed stably with a DNA molecule coding a molecular homolog having sufficient structural similarity to a tumor specific protein endogenously expressed in a tumor such that the molecular homolog is capable of inducing an immune response to the tumor specific protein in a subject bearing the tumor.

27. A live vaccine comprising the commensal bacteria cell of claim 26.

28. A cellular vaccine comprising the cell of claim 25, wherein the cellular vaccine is capable of inducing an immune response against a tumor specific protein endogenously expressed in a tumor, when the cellular vaccine is administering to a subject bearing the tumor.

29. A method of making a vaccine for inducing an immune response against a tumor specific protein endogenously expressed in a tumor, comprising selecting a molecular homolog having sufficient structural similarity to the tumor specific protein so as to enable the molecular homolog to induce the immune response against the tumor specific protein in the subject bearing the tumor.

30. The method of claim 29 wherein the molecular homolog is a xenogeneic homolog of the tumor specific protein.

31. The method of claim 29 wherein the molecular homolog is generated using genetic engineering.

32. The method of claim 29 wherein the molecular homolog is a DNA molecule.

33. The method of claim 29 wherein the molecular homolog is a protein molecule.

34. The method of claim 29 wherein the molecular homolog is attached to a virus as a carrier.

35. The method of claim 34 wherein the virus is either an Adenovirus or the a Lentivirus.

36. The method of claim 29 wherein the tumor specific protein is a tumor receptor.

37. The method of claim 36 wherein the tumor receptor is an epidermal growth factor receptor (EGFR).

38. The method of claim 37 wherein the structural similarity between the molecular homolog and EGFR ranges from 30-95%.

39. The method of claim 29 wherein the molecular homolog is modified by attaching thereto a nanoparticle in order to enhance the target specificity of the vaccine.

40. The method of claim 39 wherein the nanoparticle is an Adenovirus.

41. The method of claim 40 wherein the Adenovirus is modified by the peptide RGD.

42. The method of claim 29 wherein the subject is an animal.

43. The method of claim 29 wherein the subject is a human.

44. A method of inhibiting in vitro growth of tumor cells expressing a tumor specific protein endogenously, comprising incubating with the tumor cells a molecular homolog having sufficient structural similarity to the tumor specific protein such that the molecular homolog is capable of inhibiting the growth of the tumor cells, and measuring that growth of the tumor cells is inhibited.

45. A method of inhibiting formation or growth of a tumor of a subject, the tumor having a tumor specific protein endogenously expressed therein, comprising the step of administering to the subject a molecular homolog having sufficient structural similarity to the tumor specific protein so as to enable the molecular homolog to induce an immune response to the tumor specific protein.

46. The method of claim 45 wherein the molecular homolog is a xenogeneic homolog of the tumor specific protein.

47. The method of claim 45 wherein the molecular homolog is generated using genetic engineering.

48. The method of claim 45 wherein the molecular homolog is a DNA molecule.

49. The method of claim 45 wherein the molecular homolog is a protein molecule.

50. The method of claim 45 wherein the molecular homolog is attached to a virus as a carrier.

51. The method of claim 50 wherein the virus is either an Adenovirus or a Lentivirus.

52. The method of claim 45 wherein the tumor specific protein is a tumor receptor.

53. The method of claim 52 wherein the tumor receptor is an epidermal growth factor receptor (EGFR).

54. The method of claim 53 wherein the structural similarity between the molecular homolog and EGFR ranges from 30-95%.

55. The method of claim 45 wherein the molecular homolog is modified by attaching thereto a nanoparticle in order to enhance the target specificity of the vaccine.

56. The method of claim 55 wherein the nanoparticle is an Adenovirus.

57. The method of claim 56 wherein the Adenovirus is modified by the peptide RGD.

58. The method of claim 45 wherein the subject is an animal.

59. The method of claim 45 wherein the subject is a human.

60. A method of inducing regression of an existing tumor of a subject, the tumor having a tumor specific protein endogenously expressed therein, comprising the step of administering to the subject a molecular homolog having sufficient structural similarity to the tumor specific protein so as to enable the molecular homolog to induce an immune response to the tumor specific protein

61. A method of inducing cytotoxic T-lymphocyte activity specifically directed against a tumor cell expressing a tumor specific protein in a subject which is endogenously expressed in the tumor comprising administering to said subject a molecular homolog having sufficient structural similarity to the tumor specific protein so as to enable the molecular homolog to induce cytotoxic T-lymphocyte activity against the tumor specific protein in the subject bearing the tumor.

62. A method for inducing immunity against a tumor specific protein endogenously expressed in a tumor, comprising administering to a subject bearing the tumor a molecular homolog having sufficient structural similarity to the tumor specific protein so as to enable the molecular homolog to induce the immunity against the tumor specific protein.

63. The method of claim 62 wherein the molecular homolog is a xenogeneic homolog of the tumor specific protein.

64. The method of claim 62 wherein the molecular homolog is generated using genetic engineering.

65. The method of claim 62 wherein the molecular homolog is a DNA molecule.

66. The method of claim 62 wherein the molecular homolog is a protein molecule.

67. The method of claim 62 wherein the molecular homolog is attached to a virus as a carrier.

68. The method of claim 67 wherein the virus is either an Adenovirus or a Lentivirus.

69. The method of claim 62 wherein the tumor specific protein is a tumor receptor.

70. The method of claim 69 wherein the tumor receptor is an epidermal growth factor receptor (EGFR).

71. The method of claim 70 wherein the structural similarity between the molecular homolog and EGFR ranges from 30-95%.

72. The method of claim 62 wherein the molecular homolog is modified by attaching thereto a nanoparticle in order to enhance the target specificity of the vaccine.

73. The method of claim 72 wherein the nanoparticle is an Adenovirus.

74. The method of claim 73 wherein the Adenovirus is modified by the peptide RGD.

75. The method of claim 62 wherein the subject is an animal.

76. The method of claim 62 wherein the subject is a human.

77. A method of immunizing an animal against a tumor having a tumor specific protein endogenously expressed therein, comprising the step of administering to the animal a molecular homolog having sufficient structural similarity to the tumor specific protein so as to enable the molecular homolog to induce an immune response to the tumor specific protein.

78. The method of claim 77 wherein the animal is a mammal.

79. The method of claim 77 wherein the animal is an avian organism.

80. The method of claim 79 wherein the avian organism is a chicken.

81. The method of claim 77 wherein the animal is a mouse.

82. The method of claim 78 wherein the mammal is a human.

83. The method of claim 77 wherein the administering is subcutaneous.

84. The method of claim 77 wherein the administering is intradermal.

85. The method of claim 77 wherein the administering is intravenous.

86. The method of claim 77 wherein the administering is intraperitoneal.

87. The method of claim 77 wherein the molecular homolog is a xenogeneic homolog of the tumor specific protein.

88. The method of claim 77 wherein the molecular homolog is generated using genetic engineering.

89. The method of claim 77 wherein the molecular homolog is a DNA molecule.

90. The method of claim 77 wherein the molecular homolog is a protein molecule.

91. The method of claim 77 wherein the molecular homolog is attached to a virus as a carrier.

92. The method of claim 91 wherein the virus is either an Adenovirus or a Lentivirus.

93. The method of claim 77 wherein the tumor specific protein is a tumor receptor.

94. The method of claim 93 wherein the tumor receptor is an epidermal growth factor receptor (EGFR).

95. The method of claim 94 wherein the structural similarity between the molecular homolog and EGFR ranges from 30-95%.

96. The method of claim 77 wherein the molecular homolog is modified by attaching thereto a nanoparticle in order to enhance the target specificity of the vaccine.

97. The method of claim 96 wherein the nanoparticle is an Adenovirus.

98. The method of claim 97 wherein the Adenovirus is modified by the peptide RGD.
